(19) United States
(12) Patent Application Publication
(10) Pub. No.: US 2023/0250066 A1
(43) Pub. Date: Aug. 10, 2023

(54) HERBICIDAL CINNOLINE DERIVATIVES

(71) Applicant: SYNGENTA CROP PROTECTION AG, Basel (CH)

(72) Inventors: Suzanna Jane DALE, Bracknell, Berkshire (GB); Zoe Jane ANDERSON, Bracknell, Berkshire (GB); Vikas SIKERVAR, Bracknell, Berkshire (GB)

(73) Assignee: SYNGENTA CROP PROTECTION AG, Basel (CH)

(21) Appl. No.: 17/999,334

(22) PCT Filed: May 14, 2021
(86) PCT No.: PCT/EP2021/062885

§ 371 Date: Nov. 18, 2022

(30) Foreign Application Priority Data
May 19, 2020 (GB) 2007418.3
Apr. 1, 2021 (IN) 202111015684

(51) Int. Cl. C07D 237/28; C07D 405/12; A01P 13/00; A01N 43/58

(52) U.S. Cl. CPC C07D 237/28; C07D 405/12; A01P 13/00; A01N 43/58

(57) ABSTRACT

Compounds of the formula (I), wherein the substituents are as defined in claim 1. The invention further relates to herbicidal compositions which comprise a compound of Formula (I) and to the use of compounds of Formula (I) for controlling weeds, in particular in crops of useful plants.

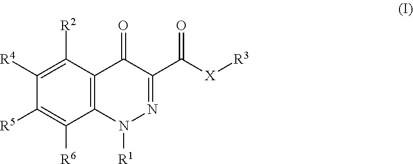

The present invention relates to herbicidal cinnoline derivatives, e.g., as active ingredients, which have herbicidal activity. The invention also relates to agrochemical compositions which comprise at least one of the cinnoline derivatives, to processes of preparation of these compounds and to uses of the cinnoline derivatives or compositions in agriculture or horticulture for controlling weeds, in particular in crops of useful plants.

EP0273325, EP0274717, and U.S. Pat. No. 5,183,891 describe cinnoline derivatives as herbicidal agents.

According to the present invention, there is provided a compound of Formula (I):

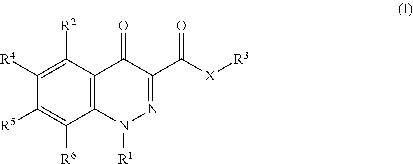

wherein

X is O, NR¹⁰ or S;

R¹ is phenyl optionally substituted with 1, 2, 3, or 4 groups, which may be the same or different, represented by R⁷;

R² is S(O)ₙC₁-C₆alkyl, S(O)ₙC₁-C₆haloalkyl, or S(O)ₙC₃-C₆cycloalkyl;

n is 0, 1 or 2;

R³ is hydrogen, C₁-C₁₂alkyl, C₁-C₆haloalkyl, cyanoC₁-C₆alkyl, C₃-C₆cycloalkyl, C₃-C₆cycloalkylC₁-C₆alkyl, C₁-C₆alkoxyC₁-C₆alkyl, C₂-C₆alkenyl, C₂-C₆haloalkenyl, C₂-C₆alkynyl, C₁-C₆alkoxycarbonylC₁-C₆alkyl, N,N-di(C₁-C₆alkyl)aminoC₁-C₆alkyl, phenyl, phenylC₁-C₁₂alkyl, benzyloxyC₁-C₆alkyl, heterocyclyl, wherein the wherein the heterocyclyl moiety is a 4-, 5- or 6-membered non-aromatic monocyclic ring comprising 1 or 2 heteroatoms individually selected from N, O and S, and wherein the phenyl and heterocyclyl moieties may be optionally substituted with 1, 2, 3 or 4 groups, which may be the same or different, represented by R⁸;

R⁴, R⁵, and R⁶ are each independently selected from hydrogen, halogen, cyano, C₁-C₆alkyl, C₁-C₆alkoxy, C₁-C₆haloalkyl, C₁-C₆haloalkoxy, C₁-C₆alkylsulfanyl, C₁-C₆alkylsulfinyl, and C₁-C₆alkylsulfonyl;

R⁷ is halogen, cyano, C₁-C₆alkyl, C₁-C₆alkoxy, C₁-C₆haloalkyl, C₁-C₆haloalkoxy, C₁-C₆alkylsulfanyl, C₁-C₆alkylsulfinyl, or C₁-C₆alkylsulfonyl; or

any two adjacent R⁷ groups together with the carbon atoms to which they are attached, may form a 5- or 6-membered heterocyclyl ring, comprising 1 or 2 heteroatoms selected from 0 and N, and wherein the heterocyclyl ring may be optionally substituted with 1, 2, 3, or 4 groups, which may be the same or different, represented by R⁹;

R⁸ and R⁹ are each independently selected from halogen, C₁-C₃alkyl, and C₁-C₃alkoxy;

R¹⁰ is hydrogen, C₁-C₃alkyl, or C₁-C₃alkoxy;

or a salt or an N-oxide thereof.

Surprisingly, it has been found that the novel compounds of Formula (I) have, for practical purposes, a very advantageous level of herbicidal activity.

According to a second aspect of the invention, there is provided an agrochemical composition comprising a herbicidally effective amount of a compound of Formula (I) according to the present invention. Such an agricultural composition may further comprise at least one additional active ingredient and/or an agrochemically-acceptable diluent or carrier.

According to a third aspect of the invention, there is provided a method of controlling weeds at a locus comprising applying to the locus a weed controlling amount of a composition comprising a compound of Formula (I).

According to a fourth aspect of the invention, there is provided the use of a compound of Formula (I) as a herbicide.

Where substituents are indicated as being “optionally substituted”, this means that they may or may not carry one or more identical or different substituents, e.g., one, two or three substituents. For example, C₁-C₈alkyl substituted by 1, 2 or 3 halogens, may include, but not be limited to, —CH₂Cl, —CHCl₂, —CCl₃, —CH₂F, —CHF₂, —CF₃, —CH₂CF₃ or —CF₂CH₃ groups. As another example, C₁-C₆alkoxy substituted by 1, 2 or 3 halogens, may include, but not limited to, CH₂ClO—, CHCl₂O—, CCl₃O—, CH₂FO—, CHF₂O—, CF₃O—, CF₃CH₂O— or CH₃CF₂O— groups.

As used herein, the term “cyano” means a —CN group.

As used herein, the term “halogen” refers to fluorine (fluoro), chlorine (chloro), bromine (bromo) or iodine (iodo).

As used herein, the term “hydroxy” means an —OH group.

As used herein, the term “C₁-C₁₂alkyl” refers to a straight or branched hydrocarbon chain radical consisting solely of carbon and hydrogen atoms, containing no unsaturation, having from one to twelve carbon atoms, and which is attached to the rest of the molecule by a single bond. “C₁-C₁₁alkyl”, “C₁-C₆alkyl”, “C₁-C₄alkyl” and “C₁-C₃alkyl” are to be construed accordingly. Examples of C₁-C₁₂alkyl include, but are not limited to, methyl, ethyl, n-propyl, and the isomers thereof, for example, iso-propyl. A “C₁-C₁₂alkylene” group refers to the corresponding definition of C₁-C₁₂alkyl, except that such radical is attached to the rest of the molecule by two single bonds. The terms “C₁-C₆alkylene”, “C₁-C₃alkylene”, and “C₁-C₂alkylene” are to be construed accordingly. Examples of C₁-C₁₂alkylene, include, but are not limited to, —CH₂—, —CH₂CH₂— and —(CH₂)₃—.

As used herein, the term “cyanoC₁-C₆alkyl” refers to a C₁-C₆alkyl radical as generally defined above substituted by one or more cyano groups, as defined above. Examples of cyanoC₁-C₆alkyl include, but are not limited to 2-cyanoethyl.

As used herein, the term “C₁-C₆haloalkyl” refers to a C₁-C₆alkyl radical as generally defined above substituted by one or more of the same or different halogen atoms. The terms “C₁-C₄haloalkyl” and “C₁-C₃haloalkyl”, are to be construed accordingly. Examples of C₁-C₆haloalkyl include, but are not limited to trifluoromethyl and 2,2,2-trifluoroethyl.

As used herein, the term “C₁-C₆alkoxy” refers to a radical of the formula —ORₐ where Rₐ is a C₁-C₆alkyl radical as generally defined above. The terms “C₁-C₄alkoxy” and “C₁-C₃alkoxy” are to be construed accordingly. Examples of C₁-C₆alkoxy include, but are not limited to, methoxy, ethoxy, 1-methylethoxy (iso-propoxy), and propoxy.

As used herein, the term “C₁-C₆haloalkoxy” refers to a C₁-C₆alkoxy radical as generally defined above substituted by one or more of the same or different halogen atoms. The terms “C₁-C₄haloalkoxy” and “C₁-C₃haloalkoxy”, are to be construed accordingly. Examples of C₁-C₆haloalkoxy include, but are not limited to trifluoromethoxy.

As used herein, the term “C₁-C₆alkoxyC₁-C₆alkyl” refers to a radical of the formula R_{b}ORₐ— wherein R_{b} is a C₁-C₆alkyl radical as generally defined above, and Rₐ is a C₁-C₆alkylene radical as generally defined above.

As used herein, the term “C₁-C₆alkoxycarbonylC₁-C₆alkyl” refers to a radical of the formula RₐOC(O)R_{b}—, wherein Rₐ is a C₁-C₆alkyl radical as generally defined above, and R_{b} is a C₁-C₆alkylene radical as generally defined above.

As used herein, the term “N,N-di(C₁-C₆alkyl)aminoC₁-C₆alkyl” refers to a radical of the formula —R_{c}N(Rₐ)(R_{b}), wherein Rₐ and R_{b} are each individually a C₁-C₆alkyl radical as generally defined above, and Re is a C₁-C₆alkylene radical as generally defined above.

As used herein, the term “C₂-C₆alkenyl” refers to a straight or branched hydrocarbon chain radical group consisting solely of carbon and hydrogen atoms, containing at least one double bond that can be of either the (E)- or (Z)-configuration, having from two to six carbon atoms, which is attached to the rest of the molecule by a single bond. The term “C₂-C₃alkenyl” is to be construed accordingly. Examples of C₂-C₆alkenyl include, but are not limited to, ethenyl (vinyl), prop-1-enyl, prop-2-enyl (allyl), but-1-enyl

As used herein, the term “C₂-C₆haloalkenyl” refers to a C₂-C₆alkenyl radical as generally defined above substituted by one or more of the same or different halogen atoms. Examples of C₂-C₆haloalkenyl include, but is not limited to 2-chloroallyl.

As used herein, the term “C₂-C₆alkynyl” refers to a straight or branched hydrocarbon chain radical group consisting solely of carbon and hydrogen atoms, containing at least one triple bond, having from two to six carbon atoms, and which is attached to the rest of the molecule by a single bond. The term “C₂-C₃alkynyl” is to be construed accordingly. Examples of C₂-C₆alkynyl include, but are not limited to, ethynyl, prop-1-ynyl, but-1-ynyl.

As used herein, the term “C₃-C₆cycloalkyl” refers to a radical which is a monocyclic saturated ring system and which contains 3 to 6 carbon atoms. The terms “C₃-C₅cycloalkyl” and “C₃-C₄cycloalkyl” are to be construed accordingly. Examples of C₃-C₆cycloalkyl include, but are not limited to, cyclopropyl, cyclobutyl, cyclopentyl and cyclohexyl.

As used herein, the term “C₃-C₆cycloalkylC₁-C₆alkyl” refers to C₃-C₆cycloalkyl ring attached to the rest of the molecule by a C₁-C₆alkylene linker as defined above.

As used herein, the term “phenylC₁-C₁₂alkyl” refers to a phenyl ring attached to the rest of the molecule by a C₁-C₁₂alkylene linker as defined above. The terms “phenylC₁-C₁₁alkyl” and “phenylC₁-C₃alkyl” are to be construed accordingly.

As used herein, the term “benzyloxyC₁-C₆alkyl” refers to a radical of the formula —RₐOR_{b}, where Rₐ is a C₁-C₆alkylene radical as generally defined above, and R_{b} is a benzyl group.

As used herein, the term “C₁-C₆alkylsulfanyl” refers to a radical of the formula —SRₐ, where Rₐ is a C₁-C₆alkyl radical as generally defined above. The terms “C₁-C₄alkylsulfanyl” and “C₁-C₃alkylsulfanyl”, are to be construed accordingly. Examples of C₁-C₆alkylsulfanyl include, but are not limited to methylsulfanyl.

As used herein, the term “C₁-C₆alkylsulfinyl” refers to a radical of the formula —S(O)Rₐ, where Rₐ is a C₁-C₆alkyl radical as generally defined above. The terms “C₁-C₄alkylsulfinyl” and “C₁-C₃alkylsulfinyl”, are to be construed accordingly. Examples of C₁-C₆alkylsulfinyl include, but are not limited to methylsulfinyl.

As used herein, the term “C₁-C₆alkylsulfonyl” refers to a radical of the formula —S(O)₂Rₐ, where Rₐ is a C₁-C₆alkyl radical as generally defined above. The terms “C₁-C₄alkylsulfonyl” and “C₁-C₃alkylsulfonyl”, are to be construed accordingly. Examples of C₁-C₆alkylsolfanyl include, but are not limited to methylsulfonyl.

As used herein, the term “heterocyclyl” refers to a stable 5- or 6-membered non-aromatic monocyclic ring which comprises 1 or 2 heteroatoms, wherein the heteroatoms are individually selected from nitrogen and oxygen. The heterocyclyl radical may be bonded to the rest of the molecule via a carbon atom or heteroatom. Examples of heterocyclyl include, but are not limited to, aziridinyl, azetidinyl, oxetanyl, tetrahydrofuryl, pyrrolidinyl, pyrazolidinyl, imidazolidnyl, piperidinyl, piperazinyl, morpholinyl, dioxolanyl.

The presence of one or more possible stereogenic elements in a compound of formula (I) means that the compounds may occur in optically isomeric forms, i.e., enantiomeric or diastereomeric forms. Also, atropisomers may occur as a result of restricted rotation about a single bond. Formula (I) is intended to include all those possible isomeric forms and mixtures thereof. The present invention includes all those possible isomeric forms and mixtures thereof for a compound of formula (I). Likewise, formula (I) is intended to include all possible tautomers. The present invention includes all possible tautomeric forms for a compound of formula (I).

In each case, the compounds of formula (I) according to the invention are in free form, in oxidized form as an N-oxide, or in salt form, e.g., an agronomically usable salt form. Salts that the compounds of Formula (I) may form with amines, including primary, secondary and tertiary amines (for example ammonia, dimethylamine and triethylamine), alkali metal and alkaline earth metal bases, transition metals or quaternary ammonium bases are preferred.

N-oxides are oxidized forms of tertiary amines or oxidized forms of nitrogen-containing heteroaromatic compounds. They are described for instance in the book “Heterocyclic N-oxides” by A. Albini and S. Pietra, CRC Press, Boca Raton (1991).

The following list provides definitions, including preferred definitions, for substituents X, R¹, R², R³, R⁴, R⁵, R⁶, R⁷, R⁸, R⁹, and R¹⁰ with reference to compounds of formula (I). For any one of these substituents, any of the definitions given below may be combined with any definition of any other substituent given below or elsewhere in this document.

X is O, N or S. Preferably, X is O or S. In one set of embodiments, X is O. In another set of embodiments, X is N. In a further set of embodiments, X is S.

R¹ is phenyl optionally substituted with 1, 2, 3, or 4 groups, which may be the same or different, represented by R⁷. Preferably, R¹ is phenyl optionally substituted with 1, 2, or 3 groups, which may be the same or different, represented by R⁷. More preferably, R¹ is phenyl optionally substituted with 1 or 2 groups, which may be the same or different, represented by R⁷. More preferably still, R¹ is phenyl optionally substituted with 1 group represented by R⁷. Even more preferably, R¹ is phenyl substituted in the para position by a single group represented by R⁷.

In one set of embodiments, R¹ is 4-(trifluoromethoxy)phenyl, 4-chlorophenyl, 2,4-dichlorophenyl, or 4-chloro-2-fluorophenyl.

In another set of embodiments, R¹ is 4-(trifluoromethoxy)phenyl or 4-chlorophenyl.

R² is S(O)ₙC₁-C₆alkyl, S(O)ₙC₁-C₆haloalkyl, or S(O)ₙC₃-C₆cycloalkyl. Preferably, R² is S(O)ₙC₁-C₄alkyl, S(O)ₙC₁-C₄haloalkyl, or S(O)ₙC₃.C₅cycloalkyl. More preferably, R² is S(O)ₙC₁-C₃alkyl, S(O)ₙC₁-C₃haloalkyl, or S(O)ₙC₃-C₄cycloalkyl. Even more preferably, R² is methylsulfanyl, methylsulfinyl, methylsulfonyl, ethylsulfanyl, ethylsulfinyl, ethylsulfonyl, n-propylsulfanyl, n-propylsulfinyl, n-propylsulfonyl, isopropylsulfanyl, isopropylsulfinyl, isopropylsulfonyl, 2,2,2-trifluoroethylsulfanyl, 2,2,2-trifluoroethylsulfinyl, 2,2,2-trifluoroethylsulfonyl, cyclopropylsulfanyl, cyclopropylsulfinyl, or cyclopropylsulfonyl. More preferably still, R² is methylsulfanyl, methylsulfinyl, methylsulfonyl, ethylsulfanyl, ethylsulfinyl, ethylsulfonyl, 2,2,2-trifluoroethylsulfanyl, 2,2,2-trifluoroethylsulfinyl, 2,2,2-trifluoroethylsulfonyl, cyclopropylsulfanyl, cyclopropylsulfinyl, or cyclopropylsulfonyl. Even more preferably, R² is methylsulfanyl, methylsulfonyl, ethylsulfanyl, ethylsulfonyl, 2,2,2-trifluoroethylsulfanyl, 2,2,2-trifluoroethylsulfonyl, cyclopropylsulfanyl, or cyclopropylsulfonyl. Even more preferably still, R² is methylsulfanyl or methylsulfonyl.

n is 0, 1 or 2. In one set of embodiments, n is 0 or 2. In another set of embodiments, n is 0. In a further set of embodiments, n is 1. In a still further set of embodiments, n is 2.

R³ is hydrogen, C₁-C₁₂alkyl, C₁-C₆haloalkyl, cyanoC₁-C₆alkyl, C₃-C₆cycloalkyl, C₃-C₆cycloalkylC₁-C₆alkyl, C₁-C₆alkoxyC₁-C₆alkyl, C₂-C₆alkenyl, C₂-C₆haloalkenyl, C₂-C₆alkynyl, C₁-C₆alkoxycarbonylC₁-C₆alkyl, N,N-di(C₁-C₆alkyl)aminoC₁-C₆alkyl, phenyl, phenylC₁-C₁₂alkyl, benzyloxyC₁-C₆alkyl, heterocyclyl, wherein the wherein the heterocyclyl moiety is a 4-, 5- or 6-membered non-aromatic monocyclic ring comprising 1 or 2 heteroatoms individually selected from N, O and S, and wherein the phenyl and heterocyclyl moieties may be optionally substituted with 1, 2, 3 or 4 groups, which may be the same or different, represented by R⁸.

Preferably, R³ is hydrogen, C₁-C₁₂alkyl, C₁-C₄haloalkyl, cyanoC₁-C₃alkyl, C₃-C₆cycloalkylC₁-C₃alkyl, C₁-C₃alkoxyC₁-C₆alkyl, C₂-C₅alkenyl, C₂-C₄haloalkenyl, C₂-C₆alkynyl, C₁-C₃alkoxycarbonylC₁-C₃alkyl, N,N-di(C₁-C₃alkyl)aminoC₁-C₃alkyl, phenylC₁-C₁₂alkyl, benzyloxyC₁-C₄alkyl, or heterocyclyl, wherein the wherein the heterocyclyl moieties are a 5- or 6-membered non-aromatic monocyclic ring comprising 1 or 2 heteroatoms individually selected from N, O and S.

More preferably, R³ is hydrogen, C₁-C₁₂alkyl, C₁-C₃haloalkyl, cyanoC₁-C₃alkyl, cyclopropylC₁-C₃alkyl, C₁-C₃alkoxyC₁-C₅alkyl, C₂-C₄alkenyl, C₂-C₃haloalkenyl, C₃-C₅alkynyl, C₁-C₂alkoxycarbonylC₁-C₂alkyl, N,N-di(methyl)aminoC₁-C₃alkyl, phenylC₁-C₁₂alkyl, benzyloxyC₁-C₄alkyl, or heterocyclyl, wherein the wherein the heterocyclyl moiety is a 5- or 6-membered non-aromatic monocyclic ring comprising 1 or 2 heteroatoms individually selected from N, O and S.

More preferably still, R³ is hydrogen, C₁-C₁₁alkyl, 2-chloroethyl, 2,2-difluoroethyl, 2-cyanoethyl, cyclopropylmethyl, 1-cyclopropylethyl, 3-methoxypropyl, 3-methoxy-3-methylbutyl, allyl, 1-methylallyl, 2-chloroallyl, prop-2-ynyl, but-3-ynyl, pent-4-ynyl, methoxycarbonylmethyl, N,N-di(methyl)aminoethyl, phenylC₃-C₉alkyl, benzyloxybutyl, or heterocyclyl, wherein the wherein the heterocyclyl moiety is a 5- or 6-membered non-aromatic monocyclic ring comprising a single oxygen atom.

Even more preferably, R³ is hydrogen, methyl, ethyl, isopropyl, isobutyl, 2,2-dimethylpropyl, n-pentyl, n-hexyl, 3,3-dimethylbutyl, n-heptyl, n-octyl, n-nonyl, n-undecyl, 2-chloroethyl, 2,2-difluoroethyl, 2-cyanoethyl, cyclopropylmethyl, 1-cyclopropylethyl, 3-methoxypropyl, 3-methoxy-3-methylbutyl, allyl, 1-methylallyl, 2-chloroallyl, prop-2-ynyl, but-3-ynyl, pent-4-ynyl, methoxycarbonylmethyl, N,N-di(methyl)aminoethyl, 9-phenylnonyl, 3-phenylpropyl, benzyloxybutyl, or tetrahydrofuran-3-yl.

In one set of embodiments, R³ is hydrogen, C₁-C₆alkyl, C₁-C₆haloalkyl, C₃-C₆cycloalkyl, C₃-C₆cycloalkylC₁-C₆alkyl, C₁-C₆alkoxyC₁-C₆alkyl, C₂-C₆alkenyl, C₂-C₆alkynyl, phenyl, or phenylC₁-C₃alkyl, wherein the phenyl moieties may be optionally substituted with 1, 2, 3 or 4 groups, which may be the same or different, represented by R⁸. More preferably, R³ is hydrogen, C₁-C₄alkyl, C₁-C₄haloalkyl, C₃-C₆cycloalkyl, C₃-C₆cycloalkylC₁-C₃alkyl, C₁-C₄alkoxyC₁-C₄alkyl, C₂-C₄alkenyl, C₂-C₄alkynyl, phenyl, or phenylC₁-C₂alkyl, wherein the phenyl moieties may be optionally substituted with 1, 2, or 3 groups, which may be the same or different, represented by R⁸. Even more preferably, R³ is hydrogen, C₁-C₄alkyl, C₁-C₄haloalkyl, C₃-C₆cycloalkyl, C₃-C₆cycloalkylC₁-C₂alkyl, C₁-C₃alkoxyC₁-C₃alkyl, C₂-C₄alkenyl, C₂-C₄alkynyl, phenyl, or phenylC₁-C₂alkyl, wherein the phenyl moieties may be optionally substituted with 1 or 2 groups, which may be the same or different, represented by R⁸. More preferably still, R³ is hydrogen or C₁-C₄alkyl. Most preferably, R³ is hydrogen, methyl or ethyl, in particular, hydrogen or methyl.

R⁴, R⁵, and R⁶ are each independently selected from hydrogen, halogen, cyano, C₁-C₆alkyl, C₁-C₆alkoxy, C₁-C₆haloalkyl, C₁-C₆haloalkoxy, C₁-C₆alkylsulfanyl, C₁-C₆alkylsulfinyl, and C₁-C₆alkylsulfonyl. Preferably, R⁴, R⁵, and R⁶ are each independently selected from hydrogen, halogen, cyano, C₁-C₄alkyl, C₁-C₄alkoxy, C₁-C₄haloalkyl, C₁-C₄haloalkoxy, C₁-C₄alkylsulfanyl, C₁-C₄alkylsulfinyl, and C₁-C₄alkylsulfonyl. More preferably, R⁴, R⁵, and R⁶ are each independently selected from hydrogen, halogen, cyano, C₁-C₄alkyl, C₁-C₃alkoxy, C₁-C₃haloalkyl, C₁-C₃haloalkoxy, C₁-C₃alkylsulfanyl, C₁-C₃alkylsulfinyl, and C₁-C₃alkylsulfonyl. More preferably still, R⁴, R⁵, and R⁶ are each independently selected from hydrogen, fluoro, bromo, cyano, C₁-C₄alkyl, methoxy, ethoxy, trifluoromethyl, trifluoromethoxy, methylsulfanyl, and methylsulfonyl. Even more preferably, R⁴, R⁵, and R⁶ are each independently selected from hydrogen, fluoro, bromo, cyano, methyl, isopropyl, isobutyl, methoxy, and trifluoromethyl. More preferably still, R⁴, R⁵, and R⁶ are each independently selected from hydrogen, fluoro, bromo, cyano, methyl, isobutyl, methoxy, and trifluoromethyl. Even more preferably still, R⁴, R⁵, and R⁶ are each independently selected from hydrogen, fluoro, bromo, cyano, methyl, isobutyl, and methoxy.

In one set of embodiments, R⁴ and R⁵ are each independently selected from hydrogen, fluoro, bromo, cyano, methyl, isobutyl, methoxy, and trifluoromethyl, and R⁶ is hydrogen. In another set of embodiments, R⁴ and R⁵ are each independently selected from hydrogen, fluoro, bromo, cyano, methyl, isobutyl, and methoxy, and R⁶ is hydrogen. In a further set of embodiments, R⁴, R⁵, and R⁶ are all hydrogen.

In another preferred set of embodiments, R⁴ and R⁵ are each independently selected from hydrogen, fluoro, bromo, methyl, isobutyl, methoxy, and trifluoromethyl, and R⁶ is hydrogen. In another set of embodiments, R⁴ and R⁵ are each independently selected from hydrogen, fluoro, bromo, methyl, isobutyl, and methoxy, and R⁶ is hydrogen.

R⁷ is halogen, cyano, C₁-C₆alkyl, C₁-C₆alkoxy, C₁-C₆haloalkyl, C₁-C₆haloalkoxy, C₁-C₆alkylsulfanyl, C₁-C₆alkylsulfinyl, or C₁-C₆alkylsulfonyl; or

any two adjacent R⁷ groups together with the carbon atoms to which they are attached, may form a 5- or 6-membered heterocyclyl ring, comprising 1 or 2 heteroatoms selected from O and N, and wherein the heterocyclyl ring may be optionally substituted with 1, 2, 3, or 4 groups, which may be the same or different, represented by R⁹.

Preferably, R⁷ is halogen, cyano, C₁-C₃alkyl, C₁-C₃alkoxy, C₁-C₃haloalkyl, C₁-C₃haloalkoxy, C₁-C₃alkylsulfanyl, C₁-C₃alkylsulfinyl, or C₁-C₃alkylsulfonyl; or

any two adjacent R⁷ groups together with the carbon atoms to which they are attached, may form a 5- or 6-membered heterocyclyl ring, comprising 1 or 2 heteroatoms selected from O and N, and wherein the heterocyclyl ring may be optionally substituted with 1, 2 or 3 groups, which may be the same or different, represented by R⁹.

More preferably, R⁷ is halogen, cyano, C₁-C₃alkyl, C₁-C₃alkoxy, C₁-C₃haloalkyl, C₁-C₃haloalkoxy, C₁-C₃alkylsulfanyl, C₁-C₃alkylsulfinyl, or C₁-C₃alkylsulfonyl.

Even more preferably, R⁷ is fluoro, bromo, chloro, cyano, methyl, ethyl, isopropyl, isobutyl, methoxy, ethoxy, trifluoromethyl, trifluoromethoxy, methylsulfanyl, methylsulfinyl, or methylsulfonyl; or

any two adjacent R⁷ groups together with the carbon atoms to which they are attached, may form a 5- or 6-membered heterocyclyl ring, comprising 1 or 2 heteroatoms selected from O and N, and wherein the heterocyclyl ring may be optionally substituted with 1 or 2 groups, which may be the same or different, represented by R⁹. Even more preferably, R⁷ is fluoro, bromo, chloro, cyano, methyl, methoxy, trifluoromethyl, or trifluoromethoxy. Even more preferably still, R⁷ is fluoro, chloro or trifluoromethoxy. More preferably still, R⁷ is chloro or trifluoromethoxy.

In one set of embodiments, R⁷ is halogen or C₁-C₃haloalkoxy.

R⁸ and R⁹ are each independently selected from halogen, C₁-C₃alkyl, and C₁-C₃alkoxy. Preferably, R⁸ and R⁹ are each independently selected from chloro, bromo, fluoro, methyl, and methoxy.

R¹⁰ is hydrogen, C₁-C₃alkyl, or C₁-C₃alkoxy. Preferably, R¹⁰ is hydrogen, methyl, or methoxy. More preferably, R¹⁰ is hydrogen.

In a compound of formula (I) according to the present invention, preferably:
X is O;
R¹ is phenyl optionally substituted with 1 group represented by R⁷;
R² is S(O)ₙC₁-C₃alkyl, S(O)ₙC₁-C₃haloalkyl, or S(O)ₙC₃-C₄cycloalkyl
R³ is hydrogen or C₁-C₄alkyl;
R⁴, R⁵, and R⁶ are each independently selected from hydrogen, fluoro, bromo, cyano, methyl, isobutyl, methoxy, and trifluoromethyl; and
R⁷ is fluoro, bromo, chloro, cyano, methyl, methoxy, trifluoromethyl, or trifluoromethoxy.

In another set of embodiments, X is 0;
R¹ is phenyl optionally substituted with 1 group represented by R⁷;
R² is S(O)ₙC₁-C₃alkyl, S(O)nCl-C₃haloalkyl, or S(O)ₙC₃-C₄cycloalkyl
R³ is hydrogen, methyl, or ethyl;
R⁴ and R⁵ are each independently selected from hydrogen, fluoro, bromo, cyano, methyl, isobutyl, methoxy, and trifluoromethyl;
R⁶ is hydrogen; and
R⁷ is fluoro, bromo, chloro, cyano, methyl, methoxy, trifluoromethyl, or trifluoromethoxy.

In a further set of embodiments,
X is 0;
R¹ is 4-(trifluoromethoxy)phenyl or 4-chlorophenyl;
R² is methylsulfanyl or methylsulfonyl;
R³ is hydrogen or methyl;
R⁴, R⁵, and R⁶ are all hydrogen.

In a particularly preferred embodiment, the compound of Formula (I) is selected from: 5-methylsulfonyl-4-oxo-1-[4-(trifluoromethoxy)phenyl]cinnoline-3-carboxylic acid (compound P2), methyl 5-methylsulfonyl-4-oxo-1-[4-(trifluoromethoxy)phenyl]cinnoline-3-carboxylate (compound P3), 1-(4-chlorophenyl)-5-methylsulfonyl-4-oxo-cinnoline-3-carboxylic acid (compound P5), 6-methyl-5-methylsulfonyl-4-oxo-1-[4-(trifluoromethoxy) phenyl]cinnoline-3-carboxylic acid (compound P6), 7-methyl-5-methylsulfonyl-4-oxo-1-[4-(trifluoromethoxy) phenyl]cinnoline-3-carboxylic acid (compound P7), ethyl 6-methyl-5-methylsulfonyl-4-oxo-1-[4-(trifluoromethoxy)phenyl]cinnoline-3-carboxylate (compound P8), ethyl 7-methyl-5-methylsulfonyl-4-oxo-1-[4-(trifluoromethoxy)phenyl]cinnoline-3-carboxylate (compound P9), ethyl 6-bromo-5-methylsulfonyl-4-oxo-1-[4-(trifluoromethoxy)phenyl]cinnoline-3-carboxylate (compound P10), ethyl 7-bromo-5-methylsulfonyl-4-oxo-1-[4-(trifluoromethoxy)phenyl]cinnoline-3-carboxylate (compound P11), 6-bromo-5-methylsulfonyl-4-oxo-1-[4-(trifluoromethoxy)phenyl]cinnoline-3-carboxylic acid (compound P12), 7-bromo-5-methylsulfonyl-4-oxo-1-[4-(trifluoromethoxy)phenyl]cinnoline-3-carboxylic acid (compound P13), 7-isobutyl-5-methylsulfonyl-4-oxo-1-[4-(trifluoromethoxy)phenyl]cinnoline-3-carboxylic acid (compound P14), 6-isobutyl-5-methylsulfonyl-4-oxo-1-[4-(trifluoromethoxy)phenyl]cinnoline-3-carboxylic acid (compound P15), 6-methoxy-5-methylsulfonyl-4-oxo-1-[4-(trifluoromethoxy)phenyl]cinnoline-3-carboxylic acid (compound P18), 7-methoxy-5-methylsulfonyl-4-oxo-1-[4-(trifluoromethoxy)phenyl]cinnoline-3-carboxylic acid (compound P19), 7-fluoro-5-methylsulfonyl-4-oxo-1-[4-(trifluoromethoxy)phenyl]cinnoline-3-carboxylic acid (compound P21), ethyl 5-ethylsulfonyl-4-oxo-1-[4-(trifluoromethoxy)phenyl]cinnoline-3-carboxylate (compound P22), ethyl 5-ethylsulfanyl-4-oxo-1-[4-(trifluoromethoxy)phenyl]cinnoline-3-carboxylate (compound P23), ethyl 5-ethylsulfinyl-4-oxo-1-[4-(trifluoromethoxy)phenyl]cinnoline-3-carboxylate (compound P24), 5-ethylsulfinyl-4-oxo-1-[4-(trifluoromethoxy)phenyl]cinnoline-3-carboxylic acid (compound P25), 5-ethylsulfanyl-4-oxo-1-[4-(trifluoromethoxy)phenyl]cinnoline-3-carboxylic acid (compound P26), 5-ethylsulfonyl-4-oxo-1-[4-(trifluoromethoxy)phenyl]cinnoline-3-carboxylic acid (compound P27), ethyl 5-methylsulfinyl-4-oxo-1-[4-(trifluoromethoxy)phenyl]cinnoline-3-carboxylate (compound P28), 5-cyclopropylsulfanyl-4-oxo-1-[4-(trifluoromethoxy)phenyl]cinnoline-3-carboxylic acid (compound P30), ethyl 5-cyclopropylsulfinyl-4-oxo-1-[4-(trifluoromethoxy)phenyl]cinnoline-3-carboxylate (compound P32), ethyl 5-cyclopropylsulfanyl-4-oxo-1-[4-(trifluoromethoxy)phenyl]cinnoline-3-carboxylate (compound P33), 4-oxo-5-(2,2,2-trifluoroethylsulfonyl)-1-[4-(trifluoromethoxy)phenyl]cinnoline-3-carboxylic acid (compound P35), 4-oxo-5-(2,2,2-trifluoroethylsulfinyl)-1-[4-(trifluoromethoxy)phenyl]cinnoline-3-carboxylic acid (compound P37), ethyl 4-oxo-5-(2,2,2-trifluoroethylsulfinyl)-1-[4-(trifluoromethoxy)phenyl]cinnoline-3-carboxylate (compound P38), ethyl 4-oxo-5-(2,2,2-trifluoroethylsulfanyl)-1-[4-(trifluoromethoxy)phenyl]cinnoline-3-carboxylate (compound P39), ethyl 4-oxo-5-(2,2,2-trifluoroethylsulfonyl)-1-[4-(trifluoromethoxy)phenyl]cinnoline-3-carboxylate (compound P40), 1-(4-chloro-2-fluoro-phenyl)-5-methylsulfonyl-4-oxo-cinnoline-3-carboxylic acid (compound P41), ethyl 1-(4-chloro-2-fluoro-phenyl)-5-methylsulfonyl-4-oxo-cinnoline-3-carboxylate (compound P42), 1-(2,4-dichlorophenyl)-5-methylsulfonyl-4-oxo-cinnoline-3-carboxylic acid (compound P43), ethyl 1-(2,4-dichlorophenyl)-5-methylsulfonyl-4-oxo-cinnoline-3-carboxylate (compound P44), ethyl 6-isobutyl-5-methylsulfonyl-4-oxo-1-[4-(trifluoromethoxy)phenyl]cinnoline-3-carboxylate (compound P45), ethyl 7-isobutyl-5-methylsulfonyl-4-oxo-1-[4-(trifluoromethoxy)phenyl]cinnoline-3-carboxylate (compound P46), ethyl 6-cyano-5-methylsulfonyl-4-oxo-1-[4-(trifluoromethoxy)phenyl]cinnoline-3-carboxylate (compound P47), ethyl 6-methoxy-5-methylsulfonyl-4-oxo-1-[4-(trifluoromethoxy)phenyl]cinnoline-3-carboxylate (compound P49), ethyl 7-methoxy-5-methylsulfonyl-4-oxo-1-[4-(trifluoromethoxy)phenyl]cinnoline-3-carboxylate (compound P50), ethyl 7-fluoro-5-methylsulfonyl-4-oxo-1-[4-(trifluoromethoxy)phenyl]cinnoline-3-carboxylate (compound P52), ethyl 6-bromo-5-methylsulfanyl-4-oxo-1-[4-(trifluoromethoxy)phenyl]cinnoline-3-carboxylate (compound P53), ethyl 7-fluoro-5-methylsulfanyl-4-oxo-1-[4-(trifluoromethoxy)phenyl]cinnoline-3-carboxylate (compound P54), ethyl 7-cyano-5-methylsulfanyl-4-oxo-1-[4-(trifluoromethoxy)phenyl]cinnoline-3-carboxylate (compound P55), methyl 7-methoxy-5-methylsulfanyl-4-oxo-1-[4-(trifluoromethoxy)phenyl]cinnoline-3-carboxylate (compound P56), ethyl 7-methoxy-5-methylsulfanyl-4-oxo-1-[4-(trifluoromethoxy)phenyl]cinnoline-3-carboxylate (compound P57), hexyl 5-methylsulfonyl-4-oxo-1-[4-(trifluoromethoxy)phenyl]cinnoline-3-carboxylate (compound P59), undecyl 5-methylsulfonyl-4-oxo-1-[4-(trifluoromethoxy)phenyl]cinnoline-3-carboxylate (compound P60), 2-chloroethyl 5-methylsulfonyl-4-oxo-1-[4-(trifluoromethoxy)phenyl]cinnoline-3-carboxylate (compound P61), pent-4-ynyl 5-methylsulfonyl-4-oxo-1-[4-(trifluoromethoxy)phenyl]cinnoline-3-carboxylate (compound P62), cyclopropylmethyl 5-methylsulfonyl-4-oxo-1-[4-(trifluoromethoxy)phenyl]cinnoline-3-carboxylate (compound P63), 1-methylallyl 5-methylsulfonyl-4-oxo-1-[4-(trifluoromethoxy)phenyl]cinnoline-3-carboxylate (compound P64), isopropyl 5-methylsulfonyl-4-oxo-1-[4-(trifluoromethoxy)phenyl]cinnoline-3-carboxylate (compound P65), 2-chloroallyl 5-methylsulfonyl-4-oxo-1-[4-(trifluoromethoxy)phenyl]cinnoline-3-carboxylate (compound P66), 2,2-difluoroethyl 5-methylsulfonyl-4-oxo-1-[4-(trifluoromethoxy)phenyl]cinnoline-3-carboxylate (compound P67), 2,2-dimethylpropyl 5-methylsulfonyl-4-oxo-1-[4-(trifluoromethoxy)phenyl]cinnoline-3-carboxylate (compound P68), 3-methoxypropyl 5-methylsulfonyl-4-oxo-1-[4-(trifluoromethoxy)phenyl]cinnoline-3-carboxylate (compound P69), tetrahydrofuran-3-yl 5-methylsulfonyl-4-oxo-1-[4-(trifluoromethoxy)phenyl]cinnoline-3-carboxylate (compound P70), but-3-ynyl 5-methylsulfonyl-4-oxo-1-[4-(trifluoromethoxy)phenyl]cinnoline-3-carboxylate (compound P71), isobutyl 5-methylsulfonyl-4-oxo-1-[4-(trifluoromethoxy)phenyl]cinnoline-3-carboxylate (P72), 2-cyanoethyl 5-methylsulfonyl-4-oxo-1-[4-(trifluoromethoxy)phenyl]cinnoline-3-carboxylate (compound P73), 1-cyclopropylethyl 5-methylsulfonyl-4-oxo-1-[4-(trifluoromethoxy)phenyl]cinnoline-3-carboxylate(compound P74), pentyl 5-methylsulfonyl-4-oxo-1-[4-(trifluoromethoxy)phenyl]cinnoline-3-carboxylate (compound P75), 2-(dimethylamino)ethyl 5-methylsulfonyl-4-oxo-1-[4-(trifluoromethoxy)phenyl]cinnoline-3-carboxylate (compound P76), heptyl 5-methylsulfonyl-4-oxo-1-[4-(trifluoromethoxy)phenyl]cinnoline-3-carboxylate (compound P77), prop-2-ynyl 5-methylsulfonyl-4-oxo-1-[4-(trifluoromethoxy)phenyl]cinnoline-3-carboxylate (compound P78), prop-2-ynyl 5-methylsulfonyl-4-oxo-1-[4-(trifluoromethoxy)phenyl]cinnoline-3-carboxylate (compound P79), allyl 5-methylsulfonyl-4-oxo-1-[4-(trifluoromethoxy)phenyl]cinnoline-3-carboxylate (compound P80), 2-methoxy-2-oxo-ethyl) 5-methylsulfonyl-4-oxo-1-[4-(trifluoromethoxy)phenyl]cinnoline-3-carboxylate (compound P81), nonyl 5-methylsulfonyl-4-oxo-1-[4-(trifluoromethoxy)phenyl]cinnoline-3-carboxylate (compound P82), 3-phenylpropyl 5-methylsulfonyl-4-oxo-1-[4-(trifluoromethoxy)phenyl]cinnoline-3-carboxylate (compound P84), (3-methoxy-3-methyl-butyl) 5-methylsulfonyl-4-oxo-1-[4-(trifluoromethoxy)phenyl]cinnoline-3-carboxylate (compound P85), 3,3-dimethylbutyl 5-methylsulfonyl-4-oxo-1-[4-(trifluoromethoxy)phenyl]cinnoline-3-carboxylate (compound P86), 2-cyclohexylethyl 5-methylsulfonyl-4-oxo-1-[4-(trifluoromethoxy)phenyl]cinnoline-3-carboxylate (compound P87), isopentyl 5-methylsulfonyl-4-oxo-1-[4-(trifluoromethoxy)phenyl]cinnoline-3-carboxylate (compound P88), 4-benzyloxybutyl 5-methylsulfonyl-4-oxo-1-[4-(trifluoromethoxy)phenyl]cinnoline-3-carboxylate (compound P89), S-octyl 5-methylsulfonyl-4-oxo-1-[4-(trifluoromethoxy)phenyl]cinnoline-3-carbothioate (compound P90), S-isopentyl 5-methylsulfonyl-4-oxo-1-[4-(trifluoromethoxy)phenyl]cinnoline-3-carbothioate (compound P91), and S-(3-phenylpropyl) 5-methylsulfonyl-4-oxo-1-[4-(trifluoromethoxy)phenyl]cinnoline-3-carbothioate (compound P92).

In another particularly preferred embodiment, the compound of Formula (I) is selected from: 5-methylsulfonyl-4-oxo-1-[4-(trifluoromethoxy)phenyl]cinnoline-3-carboxylic acid (compound P2), methyl 5-methylsulfonyl-4-oxo-1-[4-(trifluoromethoxy)phenyl]cinnoline-3-carboxylate (compound P3), 1-(4-chlorophenyl)-5-methylsulfonyl-4-oxo-cinnoline-3-carboxylic acid (compound P5). 6-methyl-5-methylsulfonyl-4-oxo-1-[4-(trifluoromethoxy) phenyl]cinnoline-3-carboxylic acid (compound P6), 7-methyl-5-methylsulfonyl-4-oxo-1-[4-(trifluoromethoxy) phenyl]cinnoline-3-carboxylic acid (compound P7), ethyl 6-methyl-5-methylsulfonyl-4-oxo-1-[4-(trifluoromethoxy)phenyl]cinnoline-3-carboxylate (compound P8), ethyl 7-methyl-5-methylsulfonyl-4-oxo-1-[4-(trifluoromethoxy)phenyl]cinnoline-3-carboxylate (compound P9), ethyl 6-bromo-5-methylsulfonyl-4-oxo-1-[4-(trifluoromethoxy)phenyl]cinnoline-3-carboxylate (compound P10), 6-bromo-5-methylsulfonyl-4-oxo-1-30 [4-(trifluoromethoxy)phenyl]cinnoline-3-carboxylic acid (compound P12), 7-bromo-5-methylsulfonyl-4-oxo-1-[4-(trifluoromethoxy)phenyl]cinnoline-3-carboxylic acid (compound P13), 7-isobutyl-5-methylsulfonyl-4-oxo-1-[4-(trifluoromethoxy)phenyl]cinnoline-3-carboxylic acid (compound P14), 6-isobutyl-5-methylsulfonyl-4-oxo-1-[4-(trifluoromethoxy)phenyl]cinnoline-3-carboxylic acid (compound P15), 6-methoxy-5-methylsulfonyl-4-oxo-1-[4-(trifluoromethoxy)phenyl]cinnoline-3-carboxylic acid (compound P18), 7-methoxy-5-methylsulfonyl-4-oxo-1-[4-(trifluoromethoxy)phenyl]cinnoline-3-carboxylic acid (compound P19), 7-fluoro-5-methylsulfonyl-4-oxo-1-[4-(trifluoromethoxy)phenyl]cinnoline-3-carboxylic acid (compound P21), ethyl 5-ethylsulfanyl-4-oxo-1-[4-(trifluoromethoxy)phenyl]cinnoline-3-carboxylate (compound P23), 5-ethylsulfinyl-4-oxo-1-[4-(trifluoromethoxy)phenyl]cinnoline-3-carboxylic acid (compound P25), 5-ethylsulfanyl-4-oxo-1-[4-(trifluoromethoxy)phenyl]cinnoline-3-carboxylic acid (compound P26), 5-ethylsulfonyl-4-oxo-1-[4-(trifluoromethoxy)phenyl]cinnoline-3-carboxylic acid (compound P27), ethyl 5-methylsulfinyl-4-oxo-1-[4-(trifluoromethoxy)phenyl]cinnoline-3-carboxylate (compound P28), 5-cyclopropylsulfanyl-4-oxo-1-[4-(trifluoromethoxy)phenyl]cinnoline-3-carboxylic acid (compound P30), ethyl 5-cyclopropylsulfinyl-4-oxo-1-[4-(trifluoromethoxy)phenyl]cinnoline-3-carboxylate (compound P32), ethyl 5-cyclopropylsulfanyl-4-oxo-1-[4-(trifluoromethoxy)phenyl]cinnoline-3-carboxylate (compound P33), 4-oxo-5-(2,2,2-trifluoroethylsulfonyl)-1-[4-(trifluoromethoxy)phenyl]cinnoline-3-carboxylic acid (compound P35), 4-oxo-5-(2,2,2-trifluoroethylsulfinyl)-1-[4-(trifluoromethoxy)phenyl]cinnoline-3-carboxylic acid (compound P37), ethyl 4-oxo-5-(2,2,2-trifluoroethylsulfanyl)-1-[4-(trifluoromethoxy)phenyl]cinnoline-3-carboxylate (compound P39), ethyl 4-oxo-5-(2,2,2-trifluoroethylsulfonyl)-1-[4-(trifluoromethoxy)phenyl]cinnoline-3-carboxylate (compound P40), 1-(4-chloro-2-fluoro-phenyl)-5-methylsulfonyl-4-oxo-cinnoline-3-carboxylic acid (compound P41), ethyl 1-(4-chloro-2-fluoro-phenyl)-5-methylsulfonyl-4-oxo-cinnoline-3-carboxylate (compound P42), 1-(2,4-dichlorophenyl)-5-methylsulfonyl-4-oxo-cinnoline-3-carboxylic acid (compound P43), ethyl 1-(2,4-dichlorophenyl)-5-methylsulfonyl-4-oxo-cinnoline-3-carboxylate (compound P44), ethyl 7-isobutyl-5-methylsulfonyl-4-oxo-1-[4-(trifluoromethoxy)phenyl]cinnoline-3-carboxylate (compound P46), ethyl 7-methoxy-5-methylsulfonyl-4-oxo-1-[4-(trifluoromethoxy)phenyl]cinnoline-3-carboxylate (compound P50), ethyl 7-fluoro-5-methylsulfonyl-4-oxo-1-[4-(trifluoromethoxy)phenyl]cinnoline-3-carboxylate (compound P52), ethyl 6-bromo-5-methylsulfanyl-4-oxo-1-[4-(trifluoromethoxy)phenyl]cinnoline-3-carboxylate (compound P53), ethyl 7-fluoro-5-methylsulfanyl-4-oxo-1-[4-(trifluoromethoxy)phenyl]cinnoline-3-carboxylate (compound P54), ethyl 7-cyano-5-methylsulfanyl-4-oxo-1-[4-(trifluoromethoxy)phenyl]cinnoline-3-carboxylate (compound P55), ethyl 7-methoxy-5-methylsulfanyl-4-oxo-1-[4-(trifluoromethoxy)phenyl]cinnoline-3-carboxylate (compound P57), hexyl 5-methylsulfonyl-4-oxo-1-[4-(trifluoromethoxy)phenyl]cinnoline-3-carboxylate (compound P59), undecyl 5-methylsulfonyl-4-oxo-1-[4-(trifluoromethoxy)phenyl]cinnoline-3-carboxylate (compound P60), 2-chloroethyl 5-methylsulfonyl-4-oxo-1-[4-(trifluoromethoxy)phenyl]cinnoline-3-carboxylate (compound P61), pent-4-ynyl 5-methylsulfonyl-4-oxo-1-[4-(trifluoromethoxy)phenyl]cinnoline-3-carboxylate (compound P62), cyclopropylmethyl 5-methylsulfonyl-4-oxo-1-[4-(trifluoromethoxy)phenyl]cinnoline-3-carboxylate (compound P63), 1-methylallyl 5-methylsulfonyl-4-oxo-1-[4-(trifluoromethoxy)phenyl]cinnoline-3-carboxylate (compound P64), isopropyl 5-methylsulfonyl-4-oxo-1-[4-(trifluoromethoxy)phenyl]cinnoline-3-carboxylate (compound P65), 2-chloroallyl 5-methylsulfonyl-4-oxo-1-[4-(trifluoromethoxy)phenyl]cinnoline-3-carboxylate (compound P66), 2,2-difluoroethyl 5-methylsulfonyl-4-oxo-1-[4-(trifluoromethoxy)phenyl]cinnoline-3-carboxylate (compound P67), 2,2-dimethylpropyl 5-methylsulfonyl-4-oxo-1-[4-(trifluoromethoxy)phenyl]cinnoline-3-carboxylate (compound P68), 3-methoxypropyl 5-methylsulfonyl-4-oxo-1-[4-(trifluoromethoxy)phenyl]cinnoline-3-carboxylate (compound P69), tetrahydrofuran-3-yl 5-methylsulfonyl-4-oxo-1-[4-(trifluoromethoxy)phenyl]cinnoline-3-carboxylate (compound P70), but-3-ynyl 5-methylsulfonyl-4-oxo-1-[4-(trifluoromethoxy)phenyl]cinnoline-3-carboxylate (compound P71), isobutyl 5-methylsulfonyl-4-oxo-1-[4-(trifluoromethoxy)phenyl]cinnoline-3-carboxylate (P72), 2-cyanoethyl 5-methylsulfonyl-4-oxo-1-[4-(trifluoromethoxy)phenyl]cinnoline-3-carboxylate (compound P73), 1-cyclopropylethyl 5-methylsulfonyl-4-oxo-1-[4-(trifluoromethoxy)phenyl]cinnoline-3-carboxylate(compound P74), pentyl 5-methylsulfonyl-4-oxo-1-[4-(trifluoromethoxy)phenyl]cinnoline-3-carboxylate (compound P75), 2-(dimethylamino)ethyl 5-methylsulfonyl-4-oxo-1-[4-(trifluoromethoxy)phenyl]cinnoline-3-carboxylate (compound P76), heptyl 5-methylsulfonyl-4-oxo-1-[4-(trifluoromethoxy)phenyl]cinnoline-3-carboxylate (compound P77), prop-2-ynyl 5-methylsulfonyl-4-oxo-1-[4-(trifluoromethoxy)phenyl]cinnoline-3-carboxylate (compound P78), prop-2-ynyl 5-methylsulfonyl-4-oxo-1-[4-(trifluoromethoxy)phenyl]cinnoline-3-carboxylate (compound P79), allyl 5-methylsulfonyl-4-oxo-1-[4-(trifluoromethoxy)phenyl]cinnoline-3-carboxylate (compound P80), 2-methoxy-2-oxo-ethyl) 5-methylsulfonyl-4-oxo-1-[4-(trifluoromethoxy)phenyl]cinnoline-3-carboxylate (compound P81), nonyl 5-methylsulfonyl-4-oxo-1-[4-(trifluoromethoxy)phenyl]cinnoline-3-carboxylate (compound P82), 3-phenylpropyl 5-methylsulfonyl-4-oxo-1-[4-(trifluoromethoxy)phenyl]cinnoline-3-carboxylate (compound P84), (3-methoxy-3-methyl-butyl) 5-methylsulfonyl-4-oxo-1-[4-(trifluoromethoxy)phenyl]cinnoline-3-carboxylate (compound P85), 3,3-dimethylbutyl 5-methylsulfonyl-4-oxo-1-[4-(trifluoromethoxy)phenyl]cinnoline-3-carboxylate (compound P86), 2-cyclohexylethyl 5-methylsulfonyl-4-oxo-1-[4-(trifluoromethoxy)phenyl]cinnoline-3-carboxylate (compound P87), isopentyl 5-methylsulfonyl-4-oxo-1-[4-(trifluoromethoxy)phenyl]cinnoline-3-carboxylate (compound P88), 4-benzyloxybutyl 5-methylsulfonyl-4-oxo-1-[4-(trifluoromethoxy)phenyl]cinnoline-3-carboxylate (compound P89), S-octyl 5-methylsulfonyl-4-oxo-1-[4-(trifluoromethoxy)phenyl]cinnoline-3-carbothioate (compound P90), S-isopentyl 5-methylsulfonyl-4-oxo-1-[4-(trifluoromethoxy)phenyl]cinnoline-3-carbothioate (compound P91), and S-(3-phenylpropyl) 5-methylsulfonyl-4-oxo-1-[4-(trifluoromethoxy)phenyl]cinnoline-3-carbothioate (compound P92).

In a further particularly preferred embodiment, the compound of Formula (I) is selected from: 5-methylsulfonyl-4-oxo-1-[4-(trifluoromethoxy)phenyl]cinnoline-3-carboxylic acid (compound P2), methyl 5-methylsulfonyl-4-oxo-1-[4-(trifluoromethoxy)phenyl]cinnoline-3-carboxylate (compound P3), 1-(4-chlorophenyl)-5-methylsulfonyl-4-oxo-cinnoline-3-carboxylic acid (compound P5). 6-methyl-5-methylsulfonyl-4-oxo-1-[4-(trifluoromethoxy) phenyl]cinnoline-3-carboxylic acid (compound P6), 7-methyl-5-methylsulfonyl-4-oxo-1-[4-(trifluoromethoxy) phenyl]cinnoline-3-carboxylic acid (compound P7), ethyl 6-methyl-5-methylsulfonyl-4-oxo-1-[4-(trifluoromethoxy)phenyl]cinnoline-3-carboxylate (compound P8), ethyl 7-methyl-5-methylsulfonyl-4-oxo-1-[4-(trifluoromethoxy)phenyl]cinnoline-3-carboxylate (compound P9), 6-bromo-5-methylsulfonyl-4-oxo-1-[4-(trifluoromethoxy)phenyl]cinnoline-3-carboxylic acid (compound P12), 7-methoxy-5-methylsulfonyl-4-oxo-1-[4-(trifluoromethoxy)phenyl]cinnoline-3-carboxylic acid (compound P19), 7-fluoro-5-methylsulfonyl-4-oxo-1-[4-(trifluoromethoxy)phenyl]cinnoline-3-carboxylic acid (compound P21), 5-ethylsulfonyl-4-oxo-1-[4-(trifluoromethoxy)phenyl]cinnoline-3-carboxylic acid (compound P27), ethyl 5-methylsulfinyl-4-oxo-1-[4-(trifluoromethoxy)phenyl]cinnoline-3-carboxylate (compound P28), 5-cyclopropylsulfanyl-4-oxo-1-[4-(trifluoromethoxy)phenyl]cinnoline-3-carboxylic acid (compound P30), ethyl 5-cyclopropylsulfinyl-4-oxo-1-[4-(trifluoromethoxy)phenyl]cinnoline-3-carboxylate (compound P32), ethyl 5-cyclopropylsulfanyl-4-oxo-1-[4-(trifluoromethoxy)phenyl]cinnoline-3-carboxylate (compound P33), 1-(4-chloro-2-fluoro-phenyl)-5-methylsulfonyl-4-oxo-cinnoline-3-carboxylic acid (compound P41), ethyl 1-(4-chloro-2-fluoro-phenyl)-5-methylsulfonyl-4-oxo-cinnoline-3-carboxylate (compound P42), 1-(2,4-dichlorophenyl)-5-methylsulfonyl-4-oxo-cinnoline-3-carboxylic acid (compound P43), ethyl 1-(2,4-dichlorophenyl)-5-methylsulfonyl-4-oxo-cinnoline-3-carboxylate (compound P44), ethyl 7-isobutyl-5-methylsulfonyl-4-oxo-1-[4-(trifluoromethoxy)phenyl]cinnoline-3-carboxylate (compound P46), ethyl 7-methoxy-5-methylsulfonyl-4-oxo-1-[4-(trifluoromethoxy)phenyl]cinnoline-3-carboxylate (compound P50), ethyl 7-fluoro-5-methylsulfonyl-4-oxo-1-[4-(trifluoromethoxy)phenyl]cinnoline-3-carboxylate (compound P52), ethyl 7-fluoro-5-methylsulfanyl-4-oxo-1-[4-(trifluoromethoxy)phenyl]cinnoline-3-carboxylate (compound P54), ethyl 7-methoxy-5-methylsulfanyl-4-oxo-1-[4-(trifluoromethoxy)phenyl]cinnoline-3-carboxylate (compound P57), hexyl 5-methylsulfonyl-4-oxo-1-[4-(trifluoromethoxy)phenyl]cinnoline-3-carboxylate (compound P59), undecyl 5-methylsulfonyl-4-oxo-1-[4-(trifluoromethoxy)phenyl]cinnoline-3-carboxylate (compound P60), 2-chloroethyl 5-methylsulfonyl-4-oxo-1-[4-(trifluoromethoxy)phenyl]cinnoline-3-carboxylate (compound P61), pent-4-ynyl 5-methylsulfonyl-4-oxo-1-[4-(trifluoromethoxy)phenyl]cinnoline-3-carboxylate (compound P62), cyclopropylmethyl 5-methylsulfonyl-4-oxo-1-[4-(trifluoromethoxy)phenyl]cinnoline-3-carboxylate (compound P63), 1-methylallyl 5-methylsulfonyl-4-oxo-1-[4-(trifluoromethoxy)phenyl]cinnoline-3-carboxylate (compound P64), isopropyl 5-methylsulfonyl-4-oxo-1-[4-(trifluoromethoxy)phenyl]cinnoline-3-carboxylate (compound P65), 2-chloroallyl 5-methylsulfonyl-4-oxo-1-[4-(trifluoromethoxy)phenyl]cinnoline-3-carboxylate (compound P66), 2,2-difluoroethyl 5-methylsulfonyl-4-oxo-1-[4-(trifluoromethoxy)phenyl]cinnoline-3-carboxylate (compound P67), 2,2-dimethylpropyl 5-methylsulfonyl-4-oxo-1-[4-(trifluoromethoxy)phenyl]cinnoline-3-carboxylate (compound P68), 3-methoxypropyl 5-methylsulfonyl-4-oxo-1-[4-(trifluoromethoxy)phenyl]cinnoline-3-carboxylate (compound P69), tetrahydrofuran-3-yl 5-methylsulfonyl-4-oxo-1-[4-(trifluoromethoxy)phenyl]cinnoline-3-carboxylate (compound P70), but-3-ynyl 5-methylsulfonyl-4-oxo-1-[4-(trifluoromethoxy)phenyl]cinnoline-3-carboxylate (compound P71), isobutyl 5-methylsulfonyl-4-oxo-1-[4-(trifluoromethoxy)phenyl]cinnoline-3-carboxylate (P72), 2-cyanoethyl 5-methylsulfonyl-4-oxo-1-[4-(trifluoromethoxy)phenyl]cinnoline-3-carboxylate (compound P73), 1-cyclopropylethyl 5-methylsulfonyl-4-oxo-1-[4-(trifluoromethoxy)phenyl]cinnoline-3-carboxylate(compound P74), pentyl 5-methylsulfonyl-4-oxo-1-[4-(trifluoromethoxy)phenyl]cinnoline-3-carboxylate (compound P75), 2-(dimethylamino)ethyl 5-methylsulfonyl-4-oxo-1-[4-(trifluoromethoxy)phenyl]cinnoline-3-carboxylate (compound P76), heptyl 5-methylsulfonyl-4-oxo-1-[4-(trifluoromethoxy)phenyl]cinnoline-3-carboxylate (compound P77), prop-2-ynyl 5-methylsulfonyl-4-oxo-1-[4-(trifluoromethoxy)phenyl]cinnoline-3-carboxylate (compound P78), prop-2-ynyl 5-methylsulfonyl-4-oxo-1-[4-(trifluoromethoxy)phenyl]cinnoline-3-carboxylate (compound P79), allyl 5-methylsulfonyl-4-oxo-1-[4-(trifluoromethoxy)phenyl]cinnoline-3-carboxylate (compound P80), 2-methoxy-2-oxo-ethyl) 5-methylsulfonyl-4-oxo-1-[4-(trifluoromethoxy)phenyl]cinnoline-3-carboxylate (compound P81), nonyl 5-methylsulfonyl-4-oxo-1-[4-(trifluoromethoxy)phenyl]cinnoline-3-carboxylate (compound P82), 3-phenylpropyl 5-methylsulfonyl-4-oxo-1-[4-(trifluoromethoxy)phenyl]cinnoline-3-carboxylate (compound P84), (3-methoxy-3-methyl-butyl) 5-methylsulfonyl-4-oxo-1-[4-(trifluoromethoxy)phenyl]cinnoline-3-carboxylate (compound P85), 3,3-dimethylbutyl 5-methylsulfonyl-4-oxo-1-[4-(trifluoromethoxy)phenyl]cinnoline-3-carboxylate (compound P86), 2-cyclohexylethyl 5-methylsulfonyl-4-oxo-1-[4-(trifluoromethoxy)phenyl]cinnoline-3-carboxylate (compound P87), isopentyl 5-methylsulfonyl-4-oxo-1-[4-(trifluoromethoxy)phenyl]cinnoline-3-carboxylate (compound P88), 4-benzyloxybutyl 5-methylsulfonyl-4-oxo-1-[4-(trifluoromethoxy)phenyl]cinnoline-3-carboxylate (compound P89), S-octyl 5-methylsulfonyl-4-oxo-1-[4-(trifluoromethoxy)phenyl]cinnoline-3-carbothioate (compound P90), S-isopentyl 5-methylsulfonyl-4-oxo-1-[4-(trifluoromethoxy)phenyl]cinnoline-3-carbothioate (compound P91), and S-(3-phenylpropyl) 5-methylsulfonyl-4-oxo-1-[4-(trifluoromethoxy)phenyl]cinnoline-3-carbothioate (compound P92).

Compounds of the invention can be made as shown in the following schemes, in which, unless otherwise stated, the definition of each variable is as defined above for a compound of Formula (I). General methods for the production of compounds of Formula (I) are described below. Unless otherwise stated in the text, X, R¹, R², R³, R⁴, R⁵, and R⁶ are as defined hereinbefore. The starting materials used for the preparation of the compounds of the invention may be purchased from usual commercial suppliers or may be prepared by known methods. The starting materials as well as the intermediates may be purified before use in the next step by state of the art methodologies such as chromatography, crystallisation, distillation and filtration.

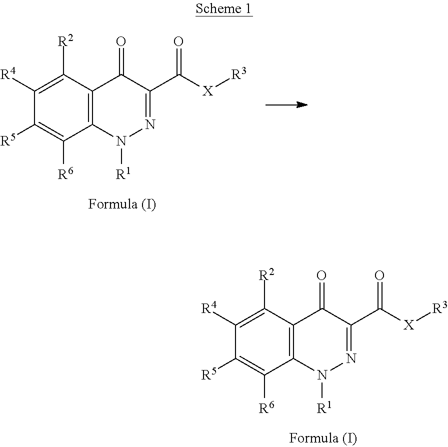

A compound of Formula (I) wherein X is oxygen and R³ is hydrogen may be prepared by hydrolysis of a compound of Formula (I) wherein R³ is not hydrogen, but any other R³ group as defined above, with a suitable base (such as sodium hydroxide or lithium hydroxide), or with a suitable acid (such as trifluoroacetic acid, hydrochloric acid, formic acid or sulfuric acid), in a suitable solvent (such as methanol, ethanol, dichloromethane, chloroform, ethyl acetate or tetrahydrofuran), with an optional co-solvent (such as water) at temperatures between 0° C. and 100° C. This is shown above in Scheme 1.

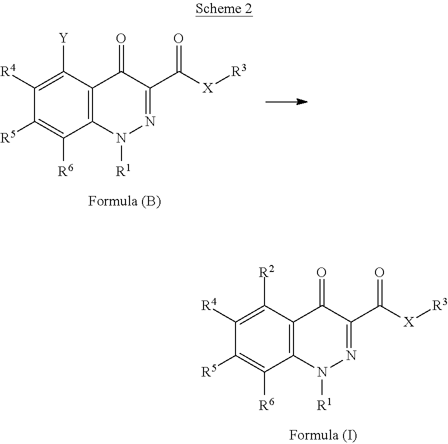

A compound of Formula (I) may be prepared from a compound of Formula (B) wherein Y is F, Cl, Br or I. In embodiments of the invention wherein R² is SO₂C₁-C₆alkyl, and Y is F compounds of Formula (I) may be prepared by reaction with an alkyl sulfinate salt (such as sodium methanesulfonate) in a suitable solvent (such as N,N-dimethylformamide, dimethyl acetamide or dimethylsulfoxide), at an elevated temperature (up to 130° C.). This is shown above in Scheme 2.

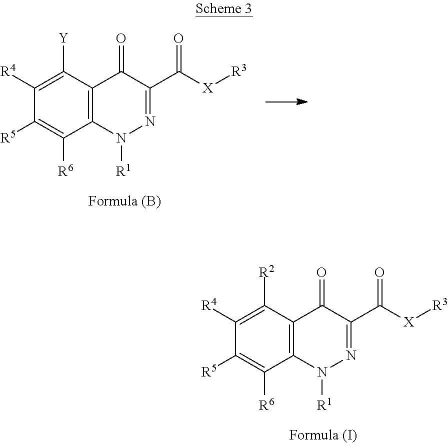

Alternatively, a compound of Formula (I) wherein R² is SC₁-C₆alkyl may be prepared from a compound of Formula (B) wherein Y is F by reaction with an alkyl thiol in the presence of a base (such as sodium hydride or a metal carbonate such as potassium carbonate), in a suitable solvent (such as N,N-dimethylformamide or N-methyl-2-pyrrolidone), at an appropriate temperature. This is shown above in Scheme 3.

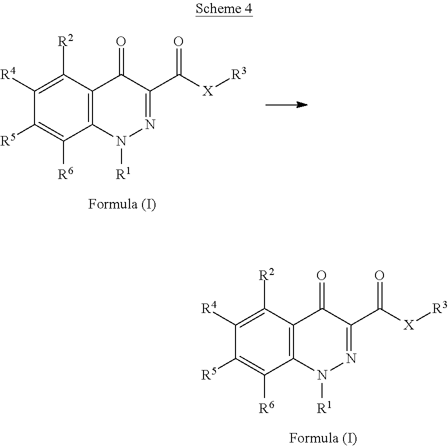

Alternatively, a compound of Formula (I) wherein R² is SO₂C₁-C₆alkyl may be prepared from a compound of Formula (I) wherein R² is SC₁-C₆alkyl or S(O)C₁-C₆alkyl by oxidation with a typical oxidant (such as oxone, sodium hypochlorite or meta-chloroperbenzoic acid), in an appropriate solvent and under standard conditions. Such methods of oxidation will be familiar to persons skilled in the art. This is shown above in Scheme 4.

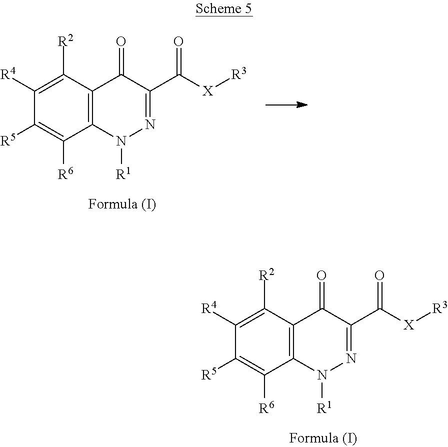

Similarly, a compound of Formula (I) wherein R² is S(O)C₁-C₅alkyl may be prepared from a compound of Formula (I) wherein R² is SC₁-C₅alkyl by oxidation with a typical oxidant (such as oxone, sodium hypochlorite or meta-chloroperbenzoic acid), in an appropriate solvent and under standard conditions. Such methods of oxidation will be familiar to persons skilled in the art. This is shown above in Scheme 5.

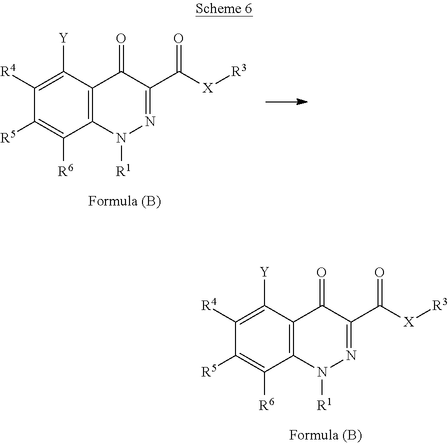

A compound of Formula (B) wherein Y is F, Cl, Br, or I, X is oxygen, and R³ is hydrogen, may be prepared by hydrolysis of a compound of Formula (B) wherein R³ is not hydrogen, but any other R³ group as defined above with a suitable base (such as sodium hydroxide or lithium hydroxide), or with a suitable acid (such as trifluoroacetic acid, hydrochloric acid, formic acid or sulfuric acid), in a suitable solvent (such as methanol, ethanol, dichloromethane, chloroform, ethyl acetate or tetrahydrofuran), with an optional co-solvent (such as water) at temperatures between 0° C. and 100° C. This is shown above in Scheme 6.

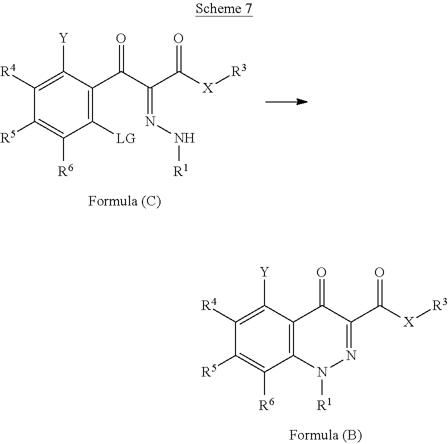

A compound of Formula (B) wherein Y is F, Cl, Br or I, and X is oxygen, may be prepared from a compound of Formula (C) optionally in the presence of a base (such as a metal hydride e.g. sodium hydride, or potassium carbonate), in a suitable solvent (such as 1,4-dioxane, tetrahydrofuran or N,N-dimethylformamide) at an elevated temperature (100° C.). This is shown above in Scheme 7.

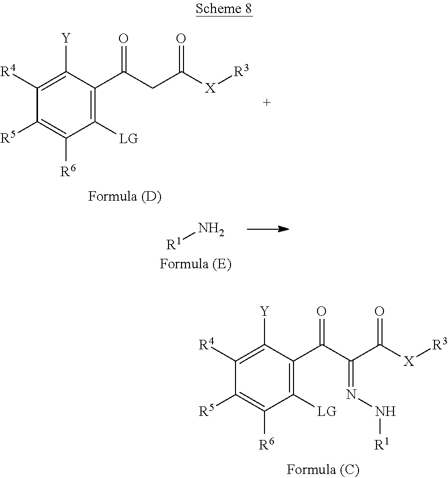

A compound of Formula (C) wherein Y is F, Cl, Br or I, and X is oxygen, may be prepared from reaction of p-keto esters of Formula (D) wherein LG is a suitable leaving group (such as F, Cl or Br), with an arene diazonium salt. The arene diazonium salts can be prepared in situ by diazotisation of anilines of Formula (E) with sodium nitrite in the presence of acid (such as hydrochloric acid), in water followed by reaction with compounds of Formula (D) in the presence of a suitable base (such as sodium or potassium acetate or potassium carbonate), in a suitable solvent (such as water, methanol or ethanol), at temperatures between 0° C. and 25° C. Compounds of Formula (E) are commercially available or may be prepared by methods familiar to persons skilled in the art. This is shown above in Scheme 8.

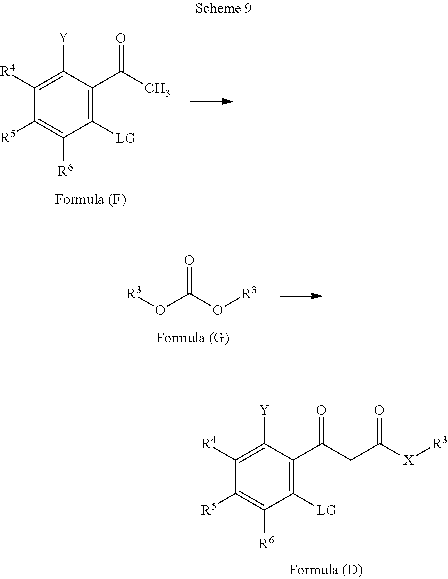

A dicarbonyl compound of Formula (D) wherein Y is F, Cl, Br or I, and X is oxygen, may be prepared from a methyl ketone compound of Formula (F) wherein LG is a suitable leaving group (such as F, Cl or Br), and a diester of Formula (G) via a Claisen condensation by treatment of the methyl ketone with a suitable base (such as potassium t-butoxide or sodium hydride), in a suitable solvent (such as tetrahydrofuran, N,N-dimethylformamide, toluene, or 1,4-dioxane), followed by reaction of the mixture with a carbonate ester (such as dimethylcarbonate or diethylcarbonate), at temperatures between 0° C. to 110° C. Compounds of Formula (F) and Formula (G) are commercially available or may be prepared by methods familiar to persons skilled in the art. This is shown above in Scheme 9.

Compounds of the invention where R⁴ is methyl can also be made by an alternative route as shown in the following schemes, in which, unless otherwise stated, the definition of each variable is as defined above for a compound of Formula (I). General methods for the production of compounds of Formula (I) are described below. Unless otherwise stated in the text, X, R¹, R², and R³, are as defined hereinbefore. The starting materials used for the preparation of the compounds of the invention may be purchased from usual commercial suppliers or may be prepared by known methods.

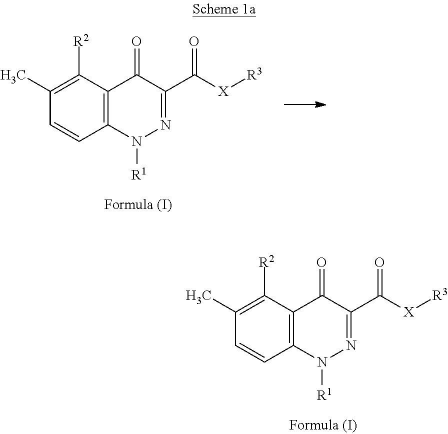

A compound of Formula (I) wherein X is oxygen and R³ is hydrogen, may be prepared by hydrolysis of a compound of Formula (I) wherein R³ is not hydrogen, but any other R³ group as defined above, with a suitable base (such as sodium hydroxide or lithium hydroxide), or with a suitable acid (such as trifluoroacetic acid, hydrochloric acid, formic acid or sulfuric acid), in a suitable solvent (such as methanol, ethanol, dichloromethane, chloroform, ethyl acetate or tetrahydrofuran), with an optional co-solvent (such as water) at temperatures between 0° C. and 100° C. This is shown above in Scheme 1a.

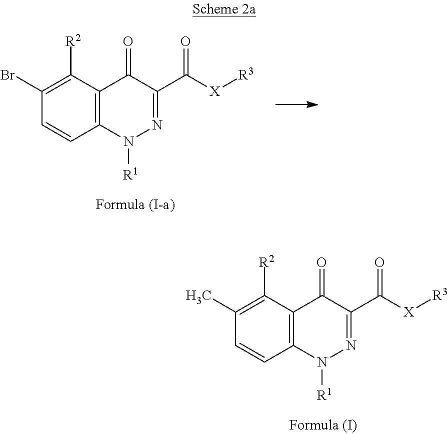

A compound of Formula (I) wherein R³ is not hydrogen, and R² is SO₂C₁-C₆alkyl, may be prepared from a compound of Formula (I-a) wherein R² is SO₂C₁-C₆alkyl, in the presence of a boroxine compound (such as trimethylboroxine) and a palladium catalyst (such as PdCl₂(dppf)), in a suitable solvent (such as 1,4-dioxane), and in the presence of a base (such as sodium carbonate) at an elevated temperature (85° C.). This is shown above in Scheme 2a.

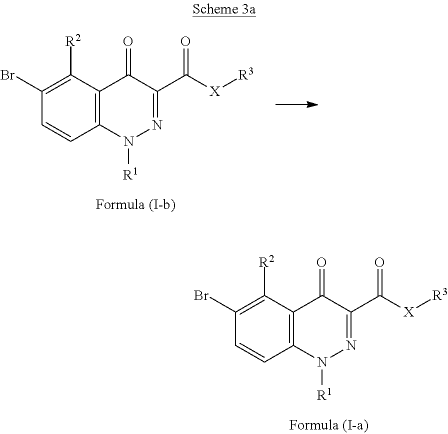

A compound of Formula (I-a) wherein R² is S(O)C₁-C₆alkyl may be prepared from a compound of Formula (I-b) wherein R² is SC₁-C₆alkyl by oxidation with a typical oxidant (such as oxone, sodium hypochlorite or meta-chloroperbenzoic acid), in an appropriate solvent and under standard conditions. Such methods of oxidation will be familiar to persons skilled in the art. This is shown above in Scheme 3a.

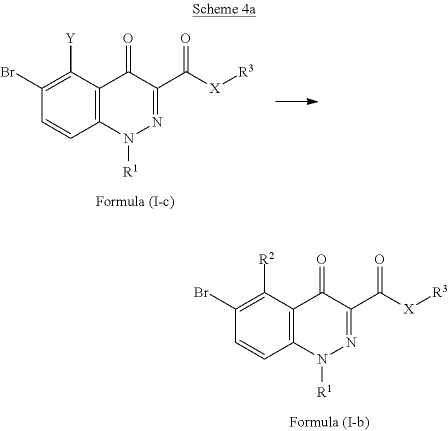

A compound of Formula (I-b) wherein R² is SC₁-C₆alkyl may be prepared from a compound of Formula (I-c) wherein Y is F, in the presence of a methanthiol salt (such as sodium methanethiol), and in a suitable solvent (such as 1,4-dioxane, tetrahydrofuran or N,N-dimethylformamide) at room temperature. This is shown above in Scheme 4a.

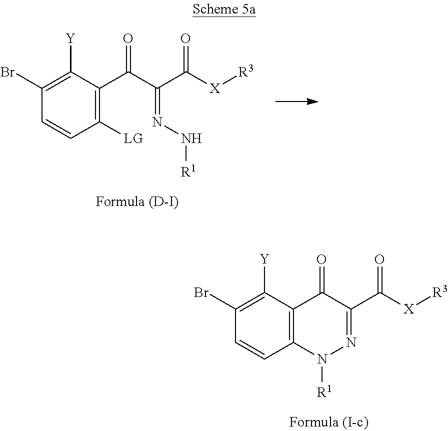

A compound of Formula (I-c) wherein Y is F, may be prepared from a compound of Formula (D-1) optionally in the presence of a base (such as a metal hydride e.g. sodium hydride, or potassium carbonate), in a suitable solvent (such as 1,4-dioxane, tetrahydrofuran or N,N-dimethylformamide) at low temperature (0° C.). This is shown above in Scheme 5a.

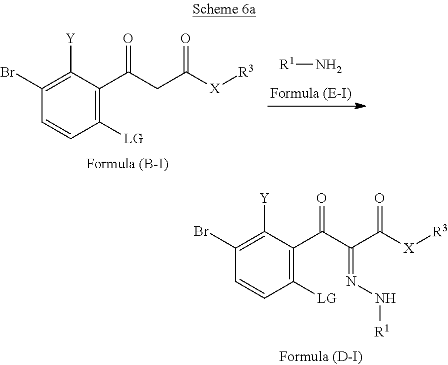

A compound of Formula (D-1) wherein Y is F, and X is oxygen, may be prepared from reaction of p-keto esters of Formula (B-1) wherein LG is a suitable leaving group (such as F, Cl or Br), with an arene diazonium salt. The arene diazonium salts can be prepared in situ by diazotisation of anilines of Formula (E) with sodium nitrite in the presence of acid (such as hydrochloric acid), in water followed by reaction with compounds of Formula (D) in the presence of a suitable base (such as sodium or potassium acetate or potassium carbonate), in a suitable solvent (such as water, methanol or ethanol), at temperatures between 0° C. and 25° C. Compounds of Formula (E-1) are commercially available or may be prepared by methods familiar to persons skilled in the art. This is shown above in Scheme 6a.

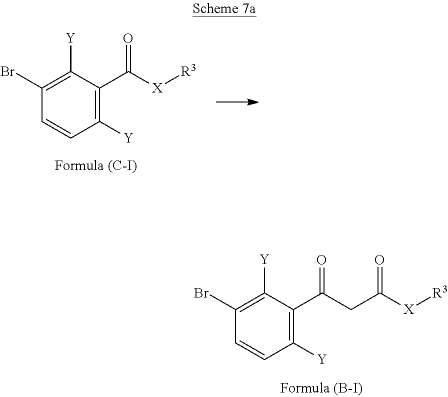

A compound of Formula (B-1) wherein Y is F, X is oxygen, and R³ is not hydrogen, may be prepared from a compound of Formula (C-1) wherein R³ is hydrogen, in the presence of magnesium chloride and an acylation reagent (such as ethyl potassium malonate), in a suitable solvent (such as tetrahydrofuran) at an elevated temperature (50° C.). Compounds of Formula (C-1) are commercially available or may be prepared by methods familiar to persons skilled in the art. This is shown above in Scheme 7a.

Similarly, compounds of the invention where R⁵ is methyl can also be made by an alternative route as shown in the following schemes, in which, unless otherwise stated, the definition of each variable is as defined above for a compound of Formula (I). General methods for the production of compounds of Formula (I) are described below. Unless otherwise stated in the text, X, R¹, R², and R³, are as defined hereinbefore. The starting materials used for the preparation of the compounds of the invention may be purchased from usual commercial suppliers or may be prepared by known methods.

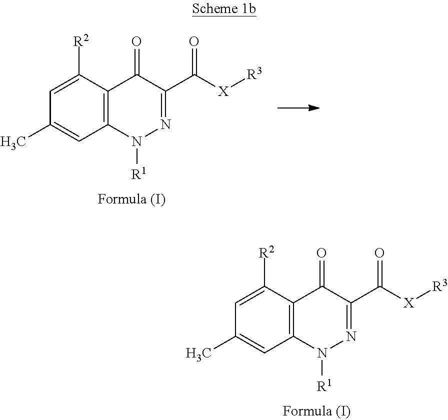

A compound of Formula (I) wherein X is oxygen and R³ is hydrogen, may be prepared by hydrolysis of a compound of Formula (I) wherein R³ is not hydrogen, but any other R³ group as defined above, with a suitable base (such as sodium hydroxide or lithium hydroxide), or with a suitable acid (such as trifluoroacetic acid, hydrochloric acid, formic acid or sulfuric acid), in a suitable solvent (such as methanol, ethanol, dichloromethane, chloroform, ethyl acetate or tetrahydrofuran), with an optional co-solvent (such as water) at temperatures between 0° C. and 100° C. This is shown above in Scheme 1b.

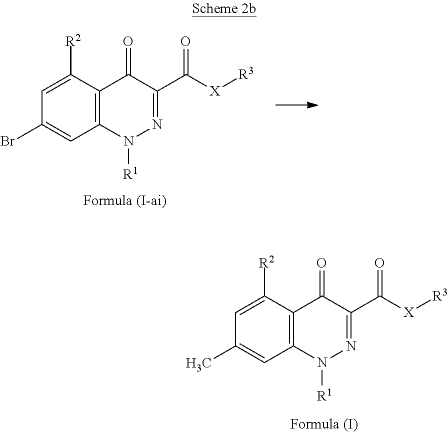

A compound of Formula (I) wherein R³ is not hydrogen, and R² is SO₂C₁-C₆alkyl, may be prepared from a compound of Formula (I-a) wherein R² is SO₂C₁-C₆alkyl, in the presence of a boroxine compound (such as trimethylboroxine) and a palladium catalyst (such as PdCl₂(dppf)), in a suitable solvent (such as 1,4-dioxane), and in the presence of a base (such as sodium carbonate) at an elevated temperature (85° C.). This is shown above in Scheme 2b.

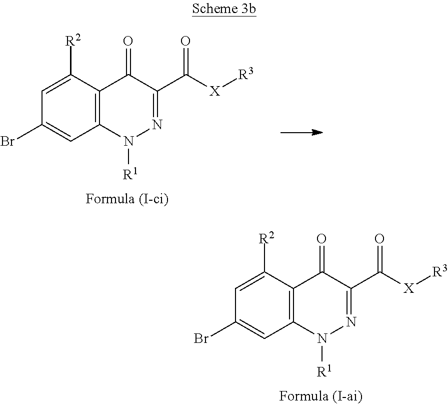

A compound of Formula (1-ai) wherein R² is S(O)C₁-C₆alkyl may be prepared from a compound of Formula (1-ci) wherein R² is SC₁-C₆alkyl by oxidation with a typical oxidant (such as oxone, sodium hypochlorite or meta-chloroperbenzoic acid), in an appropriate solvent and under standard conditions. Such methods of oxidation will be familiar to persons skilled in the art. This is shown above in Scheme 3b.

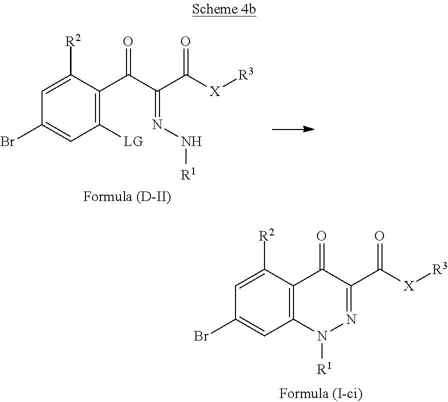

A compound of Formula (1-ci) wherein R² is SC₁-C₆alkyl, may be prepared from a compound of Formula (D-II) wherein LG is a suitable leaving group such as F, optionally in the presence of a base (such as a metal hydride e.g. sodium hydride, or potassium carbonate), in a suitable solvent (such as 1,4-dioxane, tetrahydrofuran or N,N-dimethylformamide) at an elevated temperature (100° C.). This is shown above in Scheme 4b.

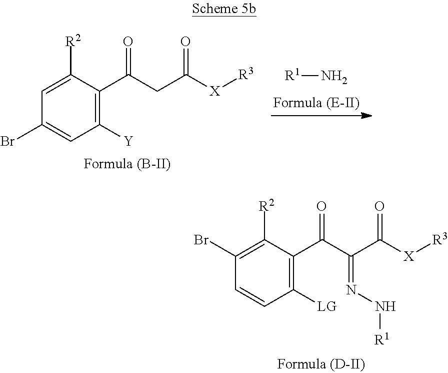

compound of formula (D-II) wherein R² is SC₁-C₆alkyl, is a suitable leaving group such as F, may be prepared from a compound of Formula (B-II), with an arene diazonium salt. The arene diazonium salts can be prepared in situ by diazotisation of anilines of Formula (E-II) with sodium nitrite in the presence of acid (such as hydrochloric acid), in water followed by reaction with compounds of Formula (B-II) in the presence of a suitable base (such as sodium or potassium acetate or potassium carbonate), in a suitable solvent (such as water, methanol or ethanol), at temperatures between 0° C. and 25° C. Compounds of Formula (E-II) are commercially available or may be prepared by methods familiar to persons skilled in the art. This is shown above in Scheme 5b.

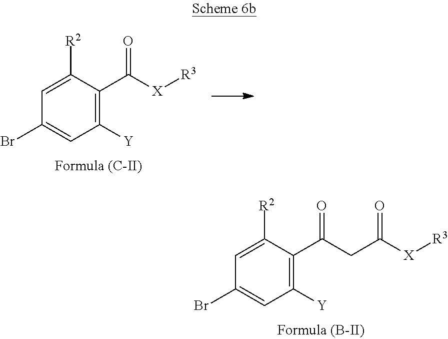

A compound of Formula (B-II) wherein R² is SC₁-C₆alkyl, Y is F, and X is oxygen, may be prepared from compounds of Formula (C-II) wherein R³ is hydrogen, in the presence of magnesium chloride and an acylation reagent (such as ethyl potassium malonate), in a suitable solvent (such as tetrahydrofuran) at an elevated temperature (80° C.). This is shown above in Scheme 6b.

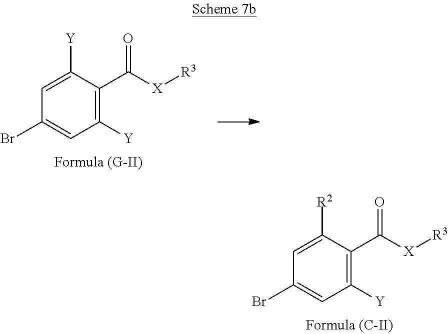

A compound of Formula (C-II) wherein R² is SC₁-C₆alkyl, X is oxygen, and R³ is hydrogen, may be prepared from a compound of Formula (G-II) wherein R³ is hydrogen, in the presence of in the presence of a methanthiol salt (such as sodium methanethiol) and a suitable base (such as lithium bis(trimethylsilyl)azanide), in a suitable solvent (such as 1,4-dioxane, tetrahydrofuran or N,N-dimethylformamide), and at an elevated temperature (80° C.). Compounds of Formula (G-II) are commercially available or may be prepared by methods familiar to persons skilled in the art. This is shown above in Scheme 7b.

The present invention still further provides a method of controlling weeds at a locus said method comprising application to the locus of a weed controlling amount of a composition comprising a compound of Formula (I). Moreover, the present invention may further provide a method of selectively controlling weeds at a locus comprising useful (crop) plants and weeds, wherein the method comprises application to the locus of a weed controlling amount of a composition according to the present invention. ‘Controlling’ means killing, reducing or retarding growth or preventing or reducing germination. It is noted that the compounds of the present invention show a much improved selectivity compared to know, structurally similar compounds. Generally the plants to be controlled are unwanted plants (weeds). ‘Locus’ means the area in which the plants are growing or will grow. The application may be applied to the locus pre-emergence and/or postemergence of the crop plant. Some crop plants may be inherently tolerant to herbicidal effects of compounds of Formula (I).

The rates of application of compounds of Formula (I) may vary within wide limits and depend on the nature of the soil, the method of application (pre- or post-emergence; seed dressing; application to the seed furrow; no tillage application etc.), the crop plant, the weed(s) to be controlled, the prevailing climatic conditions, and other factors governed by the method of application, the time of application and the target crop. The compounds of Formula I according to the invention are generally applied at a rate of from 10 to 2500 g/ha, especially from 25 to 1000 g/ha, more especially from 25 to 250 g/ha.

The application is generally made by spraying the composition, typically by tractor mounted sprayer for large areas, but other methods such as dusting (for powders), drip or drench can also be used.

The term “useful plants” is to be understood as also including useful plants that have been rendered tolerant to herbicides like bromoxynil or classes of herbicides such as, for example, 4-Hydroxyphenylpyruvate dioxygenase (HPPD) inhibitors, ALS inhibitors, for example primisulfuron, prosulfuron and trifloxysulfuron, 5-enol-pyrovyl-shikimate-3-phosphate-synthase (EPSPS) inhibitors, glutamine synthetase (GS) inhibitors or protoporphyrinogen-oxidase (PPO) inhibitors as a result of conventional methods of breeding or genetic engineering. An example of a crop that has been rendered tolerant to imidazolinones, e.g. imazamox, by conventional methods of breeding (mutagenesis) is Clearfield@ summer rape (Canola). Examples of crops that have been rendered tolerant to herbicides or classes of herbicides by genetic engineering methods include glyphosate- and glufosinate-resistant maize varieties commercially available under the trade names RoundupReady®, Herculex I® and LibertyLink®.

The term “useful plants” is to be understood as also including useful plants which have been so transformed by the use of recombinant DNA techniques that they are capable of synthesising one or more selectively acting toxins, such as are known, for example, from toxin-producing bacteria, especially those of the genus Bacillus.

Examples of such plants are: YieldGard® (maize variety that expresses a CrylA(b) toxin); YieldGard Rootworm® (maize variety that expresses a CrylllB(b1) toxin); YieldGard Plus@ (maize variety that expresses a CrylA(b) and a CrylllB(b1) toxin); Starlink® (maize variety that expresses a Cry9(c) toxin); Herculex I® (maize variety that expresses a CrylF(a2) toxin and the enzyme phosphinothricine N-acetyltransferase (PAT) to achieve tolerance to the herbicide glufosinate ammonium); NuCOTN 33B@ (cotton variety that expresses a CrylA(c) toxin); Bollgard I® (cotton variety that expresses a CrylA(c) toxin); Bollgard II@ (cotton variety that expresses a CrylA(c) and a CryllA(b) toxin); VIPCOT@ (cotton variety that expresses a VIP toxin); NewLeaf@ (potato variety that expresses a CrylllA toxin); NatureGard@ Agrisure® GT Advantage (GA21 glyphosate-tolerant trait), Agrisure® CB Advantage (Bt11 corn borer (CB) trait), Agrisure® RW (corn rootworm trait) and Protecta®.

Plant crops or seed material thereof can be both resistant to herbicides and, at the same time, resistant to insect feeding (“stacked” transgenic events). For example, seed can have the ability to express an insecticidal Cry3 protein while at the same time being tolerant to glyphosate.

Crop plants are also to be understood to include those which are obtained by conventional methods of breeding or genetic engineering and contain so-called output traits (e.g. improved storage stability, higher nutritional value and improved flavour).

The compounds of Formula (I) (or compositions comprising such) can be used to control unwanted plants (collectively, ‘weeds’). The weeds to be controlled may be both monocotyledonous species, for example Agrostis, Alopecurus, Avena, Brachiaria, Bromus, Cenchrus, Cyperus, Digitaria, Echinochloa, Eleusine, Lolium, Monochoria, Rottboellia, Sagittaria, Scirpus, Setaria and Sorghum, and dicotyledonous species, for example Abutilon, Amaranthus, Ambrosia, Chenopodium, Chrysanthemum, Conyza, Galium, Ipomoea, Nasturtium, Sida, Sinapis, Solanum, Stellaria, Veronica, Viola and Xanthium.

Compounds of Formula (I) may be used in unmodified form or, preferably, together with the adjuvants conventionally employed in the art of formulation to provide herbicidal compositions, using formulation adjuvants, such as carriers, solvents, and surface-active agents (SAA). The invention therefore further provides a herbicidal composition, comprising at least one compound Formula (I) and an agriculturally acceptable carrier and optionally an adjuvant. An agricultural acceptable carrier is for example a carrier that is suitable for agricultural use. Agricultural carriers are well known in the art.

The herbicidal compositions generally comprise from 0.1 to 99% by weight, especially from 0.1 to 95% by weight, compounds of Formula I and from 1 to 99.9% by weight of a formulation adjuvant which preferably includes from 0 to 25% by weight of a surface-active substance.

The compositions can be chosen from a number of formulation types. These include an emulsion concentrate (EC), a suspension concentrate (SC), a suspo-emulsion (SE), a capsule suspension (CS), a water dispersible granule (WG), an emulsifiable granule (EG), an emulsion, water in oil (EO), an emulsion, oil in water (EW), a micro-emulsion (ME), an oil dispersion (OD), an oil miscible flowable (OF), an oil miscible liquid (OL), a soluble concentrate (SL), an ultra-low volume suspension (SU), an ultra-low volume liquid (UL), a technical concentrate (TK), a dispersible concentrate (DC), a soluble powder (SP), a wettable powder (WP) and a soluble granule (SG). The formulation type chosen in any instance will depend upon the particular purpose envisaged and the physical, chemical, and biological properties of the compound of Formula (I).

Soluble powders (SP) may be prepared by mixing a compound of Formula (I) with one or more water-soluble inorganic salts (such as sodium bicarbonate, sodium carbonate or magnesium sulphate) or one or more water-soluble organic solids (such as a polysaccharide) and, optionally, one or more wetting agents, one or more dispersing agents or a mixture of said agents to improve water dispersibility/solubility. The mixture is then ground to a fine powder. Similar compositions may also be granulated to form water soluble granules (SG).

Wettable powders (WP) may be prepared by mixing a compound of Formula (I) with one or more solid diluents or carriers, one or more wetting agents and, preferably, one or more dispersing agents and, optionally, one or more suspending agents to facilitate the dispersion in liquids. The mixture is then ground to a fine powder. Similar compositions may also be granulated to form water dispersible granules (WG).

Granules (GR) may be formed either by granulating a mixture of a compound of Formula (I) and one or more powdered solid diluents or carriers, or from pre-formed blank granules by absorbing a compound of Formula (I) (or a solution thereof, in a suitable agent) in a porous granular material (such as pumice, attapulgite clays, fuller's earth, kieselguhr, diatomaceous earths or ground corn cobs) or by adsorbing a compound of Formula (I) (or a solution thereof, in a suitable agent) on to a hard core material (such as sands, silicates, mineral carbonates, sulphates or phosphates) and drying if necessary. Agents which are commonly used to aid absorption or adsorption include solvents (such as aliphatic and aromatic petroleum solvents, alcohols, ethers, ketones and esters) and sticking agents (such as polyvinyl acetates, polyvinyl alcohols, dextrins, sugars and vegetable oils). One or more other additives may also be included in granules (for example an emulsifying agent, wetting agent or dispersing agent).

Dispersible Concentrates (DC) may be prepared by dissolving a compound of Formula (I) in water or an organic solvent, such as a ketone, alcohol or glycol ether. These solutions may contain a surface active agent (for example to improve water dilution or prevent crystallisation in a spray tank).

Emulsifiable concentrates (EC) or oil-in-water emulsions (EW) may be prepared by dissolving a compound of Formula (I) in an organic solvent (optionally containing one or more wetting agents, one or more emulsifying agents or a mixture of said agents). Suitable organic solvents for use in ECs include aromatic hydrocarbons (such as alkylbenzenes or alkylnaphthalenes, exemplified by SOLVESSO 100, SOLVESSO 150 and SOLVESSO 200; SOLVESSO is a Registered Trade Mark), ketones (such as cyclohexanone or methylcyclohexanone) and alcohols (such as benzyl alcohol, furfuryl alcohol or butanol), N-alkylpyrrolidones (such as N-methylpyrrolidone or N-octylpyrrolidone), dimethyl amides of fatty acids (such as C₈-C₁₀ fatty acid dimethylamide) and chlorinated hydrocarbons. An EC product may spontaneously emulsify on addition to water, to produce an emulsion with sufficient stability to allow spray application through appropriate equipment.

Preparation of an EW involves obtaining a compound of Formula (I) either as a liquid (if it is not a liquid at room temperature, it may be melted at a reasonable temperature, typically below 70° C.) or in solution (by dissolving it in an appropriate solvent) and then emulsifying the resultant liquid or solution into water containing one or more SAAs, under high shear, to produce an emulsion. Suitable solvents for use in EWs include vegetable oils, chlorinated hydrocarbons (such as chlorobenzenes), aromatic solvents (such as alkylbenzenes or alkylnaphthalenes) and other appropriate organic solvents which have a low solubility in water.

Microemulsions (ME) may be prepared by mixing water with a blend of one or more solvents with one or more SAAs, to produce spontaneously a thermodynamically stable isotropic liquid formulation. A compound of Formula (I) is present initially in either the water or the solvent/SAA blend. Suitable solvents for use in MEs include those hereinbefore described for use in in ECs or in EWs. An ME may be either an oil-in-water or a water-in-oil system (which system is present may be determined by conductivity measurements) and may be suitable for mixing water-soluble and oil-soluble pesticides in the same formulation. An ME is suitable for dilution into water, either remaining as a microemulsion or forming a conventional oil-in-water emulsion.

Suspension concentrates (SC) may comprise aqueous or non-aqueous suspensions of finely divided insoluble solid particles of a compound of Formula (I). SCs may be prepared by ball or bead milling the solid compound of Formula (I) in a suitable medium, optionally with one or more dispersing agents, to produce a fine particle suspension of the compound. One or more wetting agents may be included in the composition and a suspending agent may be included to reduce the rate at which the particles settle. Alternatively, a compound of Formula (I) may be dry milled and added to water, containing agents hereinbefore described, to produce the desired end product.

Aerosol formulations comprise a compound of Formula (I) and a suitable propellant (for example n-butane). A compound of Formula (I) may also be dissolved or dispersed in a suitable medium (for example water or a water miscible liquid, such as n-propanol) to provide compositions for use in non-pressurised, hand-actuated spray pumps.

Capsule suspensions (CS) may be prepared in a manner similar to the preparation of EW formulations but with an additional polymerisation stage such that an aqueous dispersion of oil droplets is obtained, in which each oil droplet is encapsulated by a polymeric shell and contains a compound of Formula (I) and, optionally, a carrier or diluent therefor. The polymeric shell may be produced by either an interfacial polycondensation reaction or by a coacervation procedure. The compositions may provide for controlled release of the compound of Formula (I) and they may be used for seed treatment. A compound of Formula (I) may also be formulated in a biodegradable polymeric matrix to provide a slow, controlled release of the compound.

The composition may include one or more additives to improve the biological performance of the composition, for example by improving wetting, retention or distribution on surfaces; resistance to rain on treated surfaces; or uptake or mobility of a compound of Formula (I). Such additives include surface active agents (SAAs), spray additives based on oils, for example certain mineral oils or natural plant oils (such as soy bean and rape seed oil), modified plant oils such as methylated rape seed oil (MRSO), and blends of these with other bio-enhancing adjuvants (ingredients which may aid or modify the action of a compound of Formula (I).

Wetting agents, dispersing agents and emulsifying agents may be SAAs of the cationic, anionic, amphoteric or non-ionic type.

Suitable SAAs of the cationic type include quaternary ammonium compounds (for example cetyltrimethyl ammonium bromide), imidazolines and amine salts.

Suitable anionic SAAs include alkali metals salts of fatty acids, salts of aliphatic monoesters of sulphuric acid (for example sodium lauryl sulphate), salts of sulphonated aromatic compounds (for example sodium dodecylbenzenesulphonate, calcium dodecylbenzenesulphonate, butylnaphthalene sulphonate and mixtures of sodium di-isopropyl- and tri-isopropyl-naphthalene sulphonates), ether sulphates, alcohol ether sulphates (for example sodium laureth-3-sulphate), ether carboxylates (for example sodium laureth-3-carboxylate), phosphate esters (products from the reaction between one or more fatty alcohols and phosphoric acid (predominately mono-esters) or phosphorus pentoxide (predominately di-esters), for example the reaction between lauryl alcohol and tetraphosphoric acid; additionally these products may be ethoxylated), sulphosuccinamates, paraffin or olefine sulphonates, taurates, lignosulphonates and phosphates/sulphates of tristyrylphenols.

Suitable SAAs of the amphoteric type include betaines, propionates and glycinates.

Suitable SAAs of the non-ionic type include condensation products of alkylene oxides, such as ethylene oxide, propylene oxide, butylene oxide or mixtures thereof, with fatty alcohols (such as oleyl alcohol or cetyl alcohol) or with alkylphenols (such as octylphenol, nonylphenol or octylcresol); partial esters derived from long chain fatty acids or hexitol anhydrides; condensation products of said partial esters with ethylene oxide; block polymers (comprising ethylene oxide and propylene oxide); alkanolamides; simple esters (for example fatty acid polyethylene glycol esters); amine oxides (for example lauryl dimethyl amine oxide); lecithins and sorbitans and esters thereof, alkyl polyglycosides and tristyrylphenols.

Suitable suspending agents include hydrophilic colloids (such as polysaccharides, polyvinylpyrrolidone or sodium carboxymethylcellulose) and swelling clays (such as bentonite or attapulgite).

The compounds of present invention can also be used in mixture with one or more additional herbicides and/or plant growth regulators. Examples of such additional herbicides or plant growth regulators include acetochlor, acifluorfen (including acifluorfen-sodium), aclonifen, ametryn, amicarbazone, aminopyralid, aminotriazole, atrazine, beflubutamid-M, benquitrione, bensulfuron (including bensulfuron-methyl), bentazone, bicyclopyrone, bilanafos, bipyrazone, bispyribac-sodium, bixlozone, bromacil, bromoxynil, butachlor, butafenacil, carfentrazone (including carfentrazone-ethyl), cloransulam (including cloransulam-methyl), chlorimuron (including chlorimuron-ethyl), chlorotoluron, chlorsulfuron, cinmethylin, clacyfos, clethodim, clodinafop (including clodinafop-propargyl), clomazone, clopyralid, cyclopyranil, cyclopyrimorate, cyclosulfamuron, cyhalofop (including cyhalofop-butyl), 2,4-D (including the choline salt and 2-ethylhexyl ester thereof), 2,4-DB, desmedipham, dicamba (including the aluminium, aminopropyl, bis-aminopropylmethyl, choline, dichloroprop, diglycolamine, dimethylamine, dimethylammonium, potassium and sodium salts thereof) diclosulam, diflufenican, diflufenzopyr, dimethachlor, dimethenamid-P, dioxopyritrione, diquat dibromide, diuron, epyrifenacil, ethalfluralin, ethofumesate, fenoxaprop (including fenoxaprop-P-ethyl), fenoxasulfone, fenpyrazone, fenquinotrione, fentrazamide, flazasulfuron, florasulam, florpyrauxifen (including florpyrauxifen-benzyl), fluazifop (including fluazifop-P-butyl), flucarbazone (including flucarbazone-sodium), flufenacet, flumetsulam, flumioxazin, fluometuron, flupyrsulfuron (including flupyrsulfuron-methyl-sodium), fluroxypyr (including fluroxypyr-meptyl), fomesafen, foramsulfuron, glufosinate (including L-glufosinate and the ammonium salts of both), glyphosate (including the diammonium, isopropylammonium and potassium salts thereof), halauxifen (including halauxifen-methyl), haloxyfop (including haloxyfop-methyl), hexazinone, hydantocidin, imazamox (including R-imazamox), imazapic, imazapyr, imazethapyr, indaziflam, iodosulfuron (including iodosulfuron-methyl-sodium), iofensulfuron (including iofensulfuron-sodium), ioxynil, isoproturon, isoxaflutole, lancotrione, MCPA, MCPB, mecoprop-P, mesosulfuron (including mesosulfuron-methyl), mesotrione, metamitron, metazachlor, methiozolin, metolachlor, metosulam, metribuzin, metsulfuron, napropamide, nicosulfuron, norflurazon, oxadiazon, oxasulfuron, oxyfluorfen, paraquat dichloride, pendimethalin, penoxsulam, phenmedipham, picloram, pinoxaden, pretilachlor, primisulfuron-methyl, prometryne, propanil, propaquizafop, propyrisulfuron, propyzamide, prosulfocarb, prosulfuron, pyraclonil, pyraflufen (including pyraflufen-ethyl), pyrasulfotole, pyridate, pyriftalid, pyrimisulfan, pyroxasulfone, pyroxsulam, quinclorac, quinmerac, quizalofop (including quizalofop-P-ethyl and quizalofop-P-tefuryl), rimisoxafen, rimsulfuron, saflufenacil, sethoxydim, simazine, S-metalochlor, sulfentrazone, sulfosulfuron, tebuthiuron, tefuryltrione, tembotrione, terbuthylazine, terbutryn, tetflupyrolimet, thiencarbazone, thifensulfuron, tiafenacil, tolpyralate, topramezone, tralkoxydim, triafamone, triallate, triasulfuron, tribenuron (including tribenuron-methyl), triclopyr, trifloxysulfuron (including trifloxysulfuron-sodium), trifludimoxazin, trifluralin, triflusulfuron, tripyrasulfone, 3-(2-chloro-4-fluoro-5-(3-methyl-2,6-dioxo-4-trifluoromethyl-3,6-dihydropyrimidin-1(2H)-yl)phenyl)-5-methyl-4,5-dihydroisoxazole-5-carboxylic acid ethyl ester, 4-hydroxy-1-methoxy-5-methyl-3-[4-(trifluoromethyl)-2-pyridyl]imidazolidin-2-one, 4-hydroxy-1,5-dimethyl-3-[4-(trifluoromethyl)-2-pyridyl]imidazolidin-2-one, 5-ethoxy-4-hydroxy-1-methyl-3-[4-(trifluoromethyl)-2-pyridyl]imidazolidin-2-one, 4-hydroxy-1-methyl-3-[4-(trifluoromethyl)-2-pyridyl]imidazolidin-2-one, 4-hydroxy-1,5-dimethyl-3-[1-methyl-5-(trifluoromethyl)pyrazol-3-yl]imidazolidin-2-one, (4R)₁-(5-tert-butylisoxazol-3-yl)-4-ethoxy-5-hydroxy-3-methyl-imidazolidin-2-one, 4-amino-3-chloro-5-fluoro-6-(7-fluoro-1 H-indol-6-yl)pyridine-2-carboxylic acid (including agrochemically acceptable esters thereof, for example, methyl 4-amino-3-chloro-5-fluoro-6-(7-fluoro-1H-indol-6-yl)pyridine-2-carboxylate, prop-2-ynyl 4-amino-3-chloro-5-fluoro-6-(7-fluoro-1 H-indol-6-yl)pyridine-2-carboxylate and cyanomethyl 4-amino-3-chloro-5-fluoro-6-(7-fluoro-1 H-indol-6-yl)pyridine-2-carboxylate), 3-ethylsulfanyl-N-(1,3,4-oxadiazol-2-yl)-5-(trifluoromethyl)-[1,2,4]triazolo[4,3-a]pyridine-8-carboxamide, 3-(isopropylsulfanylmethyl)-N-(5-methyl-1,3,4-oxadiazol-2-yl)-5-(trifluoromethyl)-[1,2,4]triazolo[4,3-a]pyridine-8-carboxamide, 3-(isopropylsulfonylmethyl)-N-(5-methyl-1,3,4-oxadiazol-2-yl)-5-(trifluoromethyl)-[1,2,4]triazolo[4,3-a]pyridine-8-carboxamide, 3-(ethylsulfonylmethyl)-N-(5-methyl-1,3,4-oxadiazol-2-yl)-5-(trifluoromethyl)-[1,2,4]triazolo[4,3-a]pyridine-8-carboxamide, ethyl 2-[[3-[[3-chloro-5-fluoro-6-[3-methyl-2,6-dioxo-4-(trifluoromethyl)pyrimidin-1-yl]-2-pyridyl]oxy]acetate and 6-chloro-4-(2,7-dimethyl-1-naphthyl)-5-hydroxy-2-methyl-pyridazin-3-one.

The compounds or mixtures of the present invention can also be used in combination with one or more herbicide safeners. Examples of such safeners include benoxacor, cloquintocet (including cloquintocet-mexyl), cyprosulfamide, dichlormid, fenchlorazole (including fenchlorazole-ethyl), fenclorim, fluxofenim, furilazole, isoxadifen (including isoxadifen-ethyl), mefenpyr (including mefenpyr-diethyl), metcamifen and oxabetrinil.

The safeners of the compound of Formula (I) may also be in the form of esters or salts, as mentioned e.g. in The Pesticide Manual, 16^{th} Edition (BCPC), 2012. The reference to cloquintocet-mexyl also applies to a lithium, sodium, potassium, calcium, magnesium, aluminium, iron, ammonium, quaternary ammonium, sulfonium or phosphonium salt thereof as disclosed in WO 02/34048.

Preferably the mixing ratio of compound of Formula (I) to safener is from 100:1 to 1:10, especially from 20:1 to 1:1.

The compounds of Formula (I) are normally used in the form of agrochemical compositions and can be applied to the crop area or plant to be treated, simultaneously or in succession with further compounds. These further compounds can be e.g. fertilizers or micronutrient donors or other preparations, which influence the growth of plants. They can also be selective herbicides or non-selective herbicides as well as insecticides, fungicides, bactericides, nematicides, molluscicides or mixtures of several of these preparations, if desired together with further carriers, surfactants or application promoting adjuvants customarily employed in the art of formulation.

The term “locus” as used herein means fields in or on which plants are growing, or where seeds of cultivated plants are sown, or where seed will be placed into the soil. It includes soil, seeds, and seedlings, as well as established vegetation.

The term “plants” refers to all physical parts of a plant, including seeds, seedlings, saplings, roots, tubers, stems, stalks, foliage, and fruits.

The term “plant propagation material” is understood to denote generative parts of the plant, such as seeds, which can be used for the multiplication of the latter, and vegetative material, such as cuttings or tubers, for example potatoes. There may be mentioned for example seeds (in the strict sense), roots, fruits, tubers, bulbs, rhizomes, and parts of plants. Germinated plants and young plants which are to be transplanted after germination or after emergence from the soil, may also be mentioned. These young plants may be protected before transplantation by a total or partial treatment by immersion. Preferably “plant propagation material” is understood to denote seeds.

Pesticidal agents referred to herein using their common name are known, for example, from “The Pesticide Manual”, 15th Ed., British Crop Protection Council 2009.

The compounds of formula (I) may be used in unmodified form or, preferably, together with the adjuvants conventionally employed in the art of formulation. To this end, they may be conveniently formulated in known manner to emulsifiable concentrates, coatable pastes, directly sprayable or dilutable solutions or suspensions, dilute emulsions, wettable powders, soluble powders, dusts, granulates, and also encapsulations e.g. in polymeric substances. As with the type of the compositions, the methods of application, such as spraying, atomising, dusting, scattering, coating or pouring, are chosen in accordance with the intended objectives and the prevailing circumstances. The compositions may also contain further adjuvants such as stabilizers, antifoams, viscosity regulators, binders or tackifiers as well as fertilizers, micronutrient donors or other formulations for obtaining special effects.

Suitable carriers and adjuvants, e.g., for agricultural use, can be solid or liquid and are substances useful in formulation technology, e.g. natural or regenerated mineral substances, solvents, dispersants, wetting agents, tackifiers, thickeners, binders, or fertilizers. Such carriers are for example described in WO 97/33890.

The compounds of Formula (I) are normally used in the form of compositions and can be applied to the crop area or plant to be treated, simultaneously or in succession with further compounds. These further compounds can be, e.g., fertilizers or micronutrient donors or other preparations, which influence the growth of plants. They can also be selective herbicides or non-selective herbicides as well as insecticides, fungicides, bactericides, nematicides, molluscicides or mixtures of several of these preparations, if desired together with further carriers, surfactants or application promoting adjuvants customarily employed in the art of formulation.

The compound of Formula (I) may be the sole active ingredient of a composition or it may be admixed with one or more additional active ingredients such as a pesticide, fungicide, synergist, herbicide, or plant growth regulator where appropriate. An additional active ingredient may, in some cases, result in unexpected synergistic activities.

In general, the formulations include from 0.01 to 90% by weight of active agent, from 0 to 20% agriculturally acceptable surfactant and 10 to 99.99% solid or liquid formulation inerts and adjuvant(s), the active agent consisting of at least the compound of formula (I) together with component (B) and (C), and optionally other active agents, particularly microbiocides or conservatives or the like. Concentrated forms of compositions generally contain in between about 2 and 80%, preferably between about 5 and 70% by weight of active agent. Application forms of formulation may for example contain from 0.01 to 20% by weight, preferably from 0.01 to 5% by weight of active agent. Whereas commercial products will preferably be formulated as concentrates, the end user will normally employ diluted formulations.

The tables below illustrate examples of individual compounds of Formula (I) according to the invention:

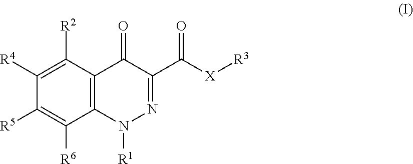

TABLE 1
Individual compounds of Formula (I) according to the invention

| | | | | Cpd | | | |
|---|---|---|---|---|---|---|---|
| No. | R⁴ | R⁵ | R² | No. | R⁴ | R⁵ | R² |
| 001 | H | H | methylsulfanyl | 385 | H | H | cyclopropylsulfanyl |
| 002 | H | F | methylsulfanyl | 386 | H | F | cyclopropylsulfanyl |
| 003 | H | Br | methylsulfanyl | 387 | H | Br | cyclopropylsulfanyl |
| 004 | H | Me | methylsulfanyl | 388 | H | Me | cyclopropylsulfanyl |
| 005 | H | iBu | methylsulfanyl | 389 | H | iBu | cyclopropylsulfanyl |
| 006 | H | CN | methylsulfanyl | 390 | H | CN | cyclopropylsulfanyl |
| 007 | H | OMe | methylsulfanyl | 391 | H | OMe | cyclopropylsulfanyl |
| 008 | H | CF₃ | methylsulfanyl | 392 | H | CF₃ | cyclopropylsulfanyl |
| 009 | F | H | methylsulfanyl | 393 | F | H | cyclopropylsulfanyl |
| 010 | F | F | methylsulfanyl | 394 | F | F | cyclopropylsulfanyl |
| 011 | F | Br | methylsulfanyl | 395 | F | Br | cyclopropylsulfanyl |
| 012 | F | Me | methylsulfanyl | 396 | F | Me | cyclopropylsulfanyl |
| 013 | F | iBu | methylsulfanyl | 397 | F | iBu | cyclopropylsulfanyl |
| 014 | F | CN | methylsulfanyl | 398 | F | CN | cyclopropylsulfanyl |
| 015 | F | OMe | methylsulfanyl | 399 | F | OMe | cyclopropylsulfanyl |
| 016 | F | CF₃ | methylsulfanyl | 400 | F | CF₃ | cyclopropylsulfanyl |
| 017 | Br | H | methylsulfanyl | 401 | Br | H | cyclopropylsulfanyl |
| 018 | Br | F | methylsulfanyl | 402 | Br | F | cyclopropylsulfanyl |
| 019 | Br | Br | methylsulfanyl | 403 | Br | Br | cyclopropylsulfanyl |
| 020 | Br | Me | methylsulfanyl | 404 | Br | Me | cyclopropylsulfanyl |
| 021 | Br | iBu | methylsulfanyl | 405 | Br | iBu | cyclopropylsulfanyl |
| 022 | Br | CN | methylsulfanyl | 406 | Br | CN | cyclopropylsulfanyl |
| 023 | Br | OMe | methylsulfanyl | 407 | Br | OMe | cyclopropylsulfanyl |
| 024 | Br | CF₃ | methylsulfanyl | 408 | Br | CF₃ | cyclopropylsulfanyl |
| 025 | Me | H | methylsulfanyl | 409 | Me | H | cyclopropylsulfanyl |
| 026 | Me | F | methylsulfanyl | 410 | Me | F | cyclopropylsulfanyl |
| 027 | Me | Br | methylsulfanyl | 411 | Me | Br | cyclopropylsulfanyl |
| 028 | Me | Me | methylsulfanyl | 412 | Me | Me | cyclopropylsulfanyl |
| 029 | Me | iBu | methylsulfanyl | 413 | Me | iBu | cyclopropylsulfanyl |
| 030 | Me | CN | methylsulfanyl | 414 | Me | CN | cyclopropylsulfanyl |
| 031 | Me | OMe | methylsulfanyl | 415 | Me | OMe | cyclopropylsulfanyl |
| 032 | Me | CF₃ | methylsulfanyl | 416 | Me | CF₃ | cyclopropylsulfanyl |
| 033 | iBu | H | methylsulfanyl | 417 | iBu | H | cyclopropylsulfanyl |
| 034 | iBu | F | methylsulfanyl | 418 | iBu | F | cyclopropylsulfanyl |
| 035 | iBu | Br | methylsulfanyl | 419 | iBu | Br | cyclopropylsulfanyl |
| 036 | iBu | Me | methylsulfanyl | 420 | iBu | Me | cyclopropylsulfanyl |
| 037 | iBu | iBu | methylsulfanyl | 421 | iBu | iBu | cyclopropylsulfanyl |
| 038 | iBu | CN | methylsulfanyl | 422 | iBu | CN | cyclopropylsulfanyl |
| 039 | iBu | OMe | methylsulfanyl | 423 | iBu | OMe | cyclopropylsulfanyl |
| 040 | iBu | CF₃ | methylsulfanyl | 424 | iBu | CF₃ | cyclopropylsulfanyl |
| 041 | CN | H | methylsulfanyl | 425 | CN | H | cyclopropylsulfanyl |
| 042 | CN | F | methylsulfanyl | 426 | CN | F | cyclopropylsulfanyl |
| 043 | CN | Br | methylsulfanyl | 427 | CN | Br | cyclopropylsulfanyl |
| 044 | CN | Me | methylsulfanyl | 428 | CN | Me | cyclopropylsulfanyl |
| 045 | CN | iBu | methylsulfanyl | 429 | CN | iBu | cyclopropylsulfanyl |
| 046 | CN | CN | methylsulfanyl | 430 | CN | CN | cyclopropylsulfanyl |
| 047 | CN | OMe | methylsulfanyl | 431 | CN | OMe | cyclopropylsulfanyl |
| 048 | CN | CF₃ | methylsulfanyl | 432 | CN | CF₃ | cyclopropylsulfanyl |
| 049 | OMe | H | methylsulfanyl | 433 | OMe | H | cyclopropylsulfanyl |
| 050 | OMe | F | methylsulfanyl | 434 | OMe | F | cyclopropylsulfanyl |
| 051 | OMe | Br | methylsulfanyl | 435 | OMe | Br | cyclopropylsulfanyl |
| 052 | OMe | Me | methylsulfanyl | 436 | OMe | Me | cyclopropylsulfanyl |
| 053 | OMe | iBu | methylsulfanyl | 437 | OMe | iBu | cyclopropylsulfanyl |
| 054 | OMe | CN | methylsulfanyl | 438 | OMe | CN | cyclopropylsulfanyl |
| 055 | OMe | OMe | methylsulfanyl | 439 | OMe | OMe | cyclopropylsulfanyl |
| 056 | OMe | CF₃ | methylsulfanyl | 440 | OMe | CF₃ | cyclopropylsulfanyl |
| 057 | CF₃ | H | methylsulfanyl | 441 | CF₃ | H | cyclopropylsulfanyl |
| 058 | CF₃ | F | methylsulfanyl | 442 | CF₃ | F | cyclopropylsulfanyl |
| 059 | CF₃ | Br | methylsulfanyl | 443 | CF₃ | Br | cyclopropylsulfanyl |
| 060 | CF₃ | Me | methylsulfanyl | 444 | CF₃ | Me | cyclopropylsulfanyl |
| 061 | CF₃ | iBu | methylsulfanyl | 445 | CF₃ | iBu | cyclopropylsulfanyl |
| 062 | CF₃ | CN | methylsulfanyl | 446 | CF₃ | CN | cyclopropylsulfanyl |
| 063 | CF₃ | OMe | methylsulfanyl | 447 | CF₃ | OMe | cyclopropylsulfanyl |
| 064 | CF₃ | CF₃ | methylsulfanyl | 448 | CF₃ | CF₃ | cyclopropylsulfanyl |
| 065 | H | H | methylsulfinyl | 449 | H | H | cyclopropylsulfinyl |
| 066 | H | F | methylsulfinyl | 450 | H | F | cyclopropylsulfinyl |
| 067 | H | Br | methylsulfinyl | 451 | H | Br | cyclopropylsulfinyl |
| 068 | H | Me | methylsulfinyl | 452 | H | Me | cyclopropylsulfinyl |
| 069 | H | iBu | methylsulfinyl | 453 | H | iBu | cyclopropylsulfinyl |
| 070 | H | CN | methylsulfinyl | 454 | H | CN | cyclopropylsulfinyl |
| 071 | H | OMe | methylsulfinyl | 455 | H | OMe | cyclopropylsulfinyl |
| 072 | H | CF₃ | methylsulfinyl | 456 | H | CF₃ | cyclopropylsulfinyl |
| 073 | F | H | methylsulfinyl | 457 | F | H | cyclopropylsulfinyl |
| 074 | F | F | methylsulfinyl | 458 | F | F | cyclopropylsulfinyl |
| 075 | F | Br | methylsulfinyl | 459 | F | Br | cyclopropylsulfinyl |
| 076 | F | Me | methylsulfinyl | 460 | F | Me | cyclopropylsulfinyl |
| 077 | F | iBu | methylsulfinyl | 461 | F | iBu | cyclopropylsulfinyl |
| 078 | F | CN | methylsulfinyl | 462 | F | CN | cyclopropylsulfinyl |
| 079 | F | OMe | methylsulfinyl | 463 | F | OMe | cyclopropylsulfinyl |
| 080 | F | CF₃ | methylsulfinyl | 464 | F | CF₃ | cyclopropylsulfinyl |
| 081 | Br | H | methylsulfinyl | 465 | Br | H | cyclopropylsulfinyl |
| 082 | Br | F | methylsulfinyl | 466 | Br | F | cyclopropylsulfinyl |
| 083 | Br | Br | methylsulfinyl | 467 | Br | Br | cyclopropylsulfinyl |
| 084 | Br | Me | methylsulfinyl | 468 | Br | Me | cyclopropylsulfinyl |
| 085 | Br | iBu | methylsulfinyl | 469 | Br | iBu | cyclopropylsulfinyl |
| 086 | Br | CN | methylsulfinyl | 470 | Br | CN | cyclopropylsulfinyl |
| 087 | Br | OMe | methylsulfinyl | 471 | Br | OMe | cyclopropylsulfinyl |
| 088 | Br | CF₃ | methylsulfinyl | 472 | Br | CF₃ | cyclopropylsulfinyl |
| 089 | Me | H | methylsulfinyl | 473 | Me | H | cyclopropylsulfinyl |
| 090 | Me | F | methylsulfinyl | 474 | Me | F | cyclopropylsulfinyl |
| 091 | Me | Br | methylsulfinyl | 475 | Me | Br | cyclopropylsulfinyl |
| 092 | Me | Me | methylsulfinyl | 476 | Me | Me | cyclopropylsulfinyl |
| 093 | Me | iBu | methylsulfinyl | 477 | Me | iBu | cyclopropylsulfinyl |
| 094 | Me | CN | methylsulfinyl | 478 | Me | CN | cyclopropylsulfinyl |
| 095 | Me | OMe | methylsulfinyl | 479 | Me | OMe | cyclopropylsulfinyl |
| 096 | Me | CF₃ | methylsulfinyl | 480 | Me | CF₃ | cyclopropylsulfinyl |
| 097 | iBu | H | methylsulfinyl | 481 | iBu | H | cyclopropylsulfinyl |
| 098 | iBu | F | methylsulfinyl | 482 | iBu | F | cyclopropylsulfinyl |
| 099 | iBu | Br | methylsulfinyl | 483 | iBu | Br | cyclopropylsulfinyl |
| 100 | iBu | Me | methylsulfinyl | 484 | iBu | Me | cyclopropylsulfinyl |
| 101 | iBu | iBu | methylsulfinyl | 485 | iBu | iBu | cyclopropylsulfinyl |
| 102 | iBu | CN | methylsulfinyl | 486 | iBu | CN | cyclopropylsulfinyl |
| 103 | iBu | OMe | methylsulfinyl | 487 | iBu | OMe | cyclopropylsulfinyl |
| 104 | iBu | CF₃ | methylsulfinyl | 488 | iBu | CF₃ | cyclopropylsulfinyl |
| 105 | CN | H | methylsulfinyl | 489 | CN | H | cyclopropylsulfinyl |
| 106 | CN | F | methylsulfinyl | 490 | CN | F | cyclopropylsulfinyl |
| 107 | CN | Br | methylsulfinyl | 491 | CN | Br | cyclopropylsulfinyl |
| 108 | CN | Me | methylsulfinyl | 492 | CN | Me | cyclopropylsulfinyl |
| 109 | CN | iBu | methylsulfinyl | 493 | CN | iBu | cyclopropylsulfinyl |
| 110 | CN | CN | methylsulfinyl | 494 | CN | CN | cyclopropylsulfinyl |
| 111 | CN | OMe | methylsulfinyl | 495 | CN | OMe | cyclopropylsulfinyl |
| 112 | CN | CF₃ | methylsulfinyl | 496 | CN | CF₃ | cyclopropylsulfinyl |
| 113 | OMe | H | methylsulfinyl | 497 | OMe | H | cyclopropylsulfinyl |
| 114 | OMe | F | methylsulfinyl | 498 | OMe | F | cyclopropylsulfinyl |
| 115 | OMe | Br | methylsulfinyl | 499 | OMe | Br | cyclopropylsulfinyl |
| 116 | OMe | Me | methylsulfinyl | 500 | OMe | Me | cyclopropylsulfinyl |
| 117 | OMe | iBu | methylsulfinyl | 501 | OMe | iBu | cyclopropylsulfinyl |
| 118 | OMe | CN | methylsulfinyl | 502 | OMe | CN | cyclopropylsulfinyl |
| 119 | OMe | OMe | methylsulfinyl | 503 | OMe | OMe | cyclopropylsulfinyl |
| 120 | OMe | CF₃ | methylsulfinyl | 504 | OMe | CF₃ | cyclopropylsulfinyl |
| 121 | CF₃ | H | methylsulfinyl | 505 | CF₃ | H | cyclopropylsulfinyl |
| 122 | CF₃ | F | methylsulfinyl | 506 | CF₃ | F | cyclopropylsulfinyl |
| 123 | CF₃ | Br | methylsulfinyl | 507 | CF₃ | Br | cyclopropylsulfinyl |
| 124 | CF₃ | Me | methylsulfinyl | 508 | CF₃ | Me | cyclopropylsulfinyl |
| 125 | CF₃ | iBu | methylsulfinyl | 509 | CF₃ | iBu | cyclopropylsulfinyl |
| 126 | CF₃ | CN | methylsulfinyl | 510 | CF₃ | CN | cyclopropylsulfinyl |
| 127 | CF₃ | OMe | methylsulfinyl | 511 | CF₃ | OMe | cyclopropylsulfinyl |
| 128 | CF₃ | CF₃ | methylsulfinyl | 512 | CF₃ | CF₃ | cyclopropylsulfinyl |
| 129 | H | H | methylsulfonyl | 513 | H | H | cyclopropylsulfonyl |
| 130 | H | F | methylsulfonyl | 514 | H | F | cyclopropylsulfonyl |
| 131 | H | Br | methylsulfonyl | 515 | H | Br | cyclopropylsulfonyl |
| 132 | H | Me | methylsulfonyl | 516 | H | Me | cyclopropylsulfonyl |
| 133 | H | iBu | methylsulfonyl | 517 | H | iBu | cyclopropylsulfonyl |
| 134 | H | CN | methylsulfony | 518 | H | CN | cyclopropylsulfonyl |
| 135 | H | OMe | methylsulfonyl | 519 | H | OMe | cyclopropylsulfonyl |
| 136 | H | CF₃ | methylsulfonyl | 520 | H | CF₃ | cyclopropylsulfonyl |
| 137 | F | H | methylsulfonyl | 521 | F | H | cyclopropylsulfonyl |
| 138 | F | F | methylsulfonyl | 522 | F | F | cyclopropylsulfonyl |
| 139 | F | Br | methylsulfonyl | 523 | F | Br | cyclopropylsulfonyl |
| 140 | F | Me | methylsulfonyl | 524 | F | Me | cyclopropylsulfonyl |
| 141 | F | iBu | methylsulfonyl | 525 | F | iBu | cyclopropylsulfonyl |
| 142 | F | CN | methylsulfonyl | 526 | F | CN | cyclopropylsulfonyl |
| 143 | F | OMe | methylsulfonyl | 527 | F | OMe | cyclopropylsulfonyl |
| 144 | F | CF₃ | methylsulfonyl | 528 | F | CF₃ | cyclopropylsulfonyl |
| 145 | Br | H | methylsulfonyl | 529 | Br | H | cyclopropylsulfonyl |
| 146 | Br | F | methylsulfonyl | 530 | Br | F | cyclopropylsulfonyl |
| 147 | Br | Br | methylsulfonyl | 531 | Br | Br | cyclopropylsulfonyl |
| 148 | Br | Me | methylsulfonyl | 532 | Br | Me | cyclopropylsulfonyl |
| 149 | Br | iBu | methylsulfonyl | 533 | Br | iBu | cyclopropylsulfonyl |
| 150 | Br | CN | methylsulfonyl | 534 | Br | CN | cyclopropylsulfonyl |
| 151 | Br | OMe | methylsulfonyl | 535 | Br | OMe | cyclopropylsulfonyl |
| 152 | Br | CF₃ | methylsulfonyl | 536 | Br | CF₃ | cyclopropylsulfonyl |
| 153 | Me | H | methylsulfonyl | 537 | Me | H | cyclopropylsulfonyl |
| 154 | Me | F | methylsulfonyl | 538 | Me | F | cyclopropylsulfonyl |
| 155 | Me | Br | methylsulfonyl | 539 | Me | Br | cyclopropylsulfonyl |
| 156 | Me | Me | methylsulfonyl | 540 | Me | Me | cyclopropylsulfonyl |
| 157 | Me | iBu | methylsulfonyl | 541 | Me | iBu | cyclopropylsulfonyl |
| 158 | Me | CN | methylsulfonyl | 542 | Me | CN | cyclopropylsulfonyl |
| 159 | Me | OMe | methylsulfonyl | 543 | Me | OMe | cyclopropylsulfonyl |
| 160 | Me | CF₃ | methylsulfonyl | 544 | Me | CF₃ | cyclopropylsulfonyl |
| 161 | iBu | H | methylsulfonyl | 545 | iBu | H | cyclopropylsulfonyl |
| 162 | iBu | F | methylsulfonyl | 546 | iBu | F | cyclopropylsulfonyl |
| 163 | iBu | Br | methylsulfonyl | 547 | iBu | Br | cyclopropylsulfonyl |
| 164 | iBu | Me | methylsulfonyl | 548 | iBu | Me | cyclopropylsulfonyl |
| 165 | iBu | iBu | methylsulfonyl | 549 | iBu | iBu | cyclopropylsulfonyl |
| 166 | iBu | CN | methylsulfonyl | 550 | iBu | CN | cyclopropylsulfonyl |
| 167 | iBu | OMe | methylsulfonyl | 551 | iBu | OMe | cyclopropylsulfonyl |
| 168 | iBu | CF₃ | methylsulfonyl | 552 | iBu | CF₃ | cyclopropylsulfonyl |
| 169 | CN | H | methylsulfonyl | 553 | CN | H | cyclopropylsulfonyl |
| 170 | CN | F | methylsulfonyl | 554 | CN | F | cyclopropylsulfonyl |
| 171 | CN | Br | methylsulfonyl | 555 | CN | Br | cyclopropylsulfonyl |
| 172 | CN | Me | methylsulfonyl | 556 | CN | Me | cyclopropylsulfonyl |
| 173 | CN | iBu | methylsulfonyl | 557 | CN | iBu | cyclopropylsulfonyl |
| 174 | CN | CN | methylsulfonyl | 558 | CN | CN | cyclopropylsulfonyl |
| 175 | CN | OMe | methylsulfonyl | 559 | CN | OMe | cyclopropylsulfonyl |
| 176 | CN | CF₃ | methylsulfonyl | 560 | CN | CF₃ | cyclopropylsulfonyl |
| 177 | OMe | H | methylsulfonyl | 561 | OMe | H | cyclopropylsulfonyl |
| 178 | OMe | F | methylsulfonyl | 562 | OMe | F | cyclopropylsulfonyl |
| 179 | OMe | Br | methylsulfonyl | 563 | OMe | Br | cyclopropylsulfonyl |
| 180 | OMe | Me | methylsulfonyl | 564 | OMe | Me | cyclopropylsulfonyl |
| 181 | OMe | iBu | methylsulfonyl | 565 | OMe | iBu | cyclopropylsulfonyl |
| 182 | OMe | CN | methylsulfonyl | 566 | OMe | CN | cyclopropylsulfonyl |
| 183 | OMe | OMe | methylsulfonyl | 567 | OMe | OMe | cyclopropylsulfonyl |
| 184 | OMe | CF₃ | methylsulfonyl | 568 | OMe | CF₃ | cyclopropylsulfonyl |
| 185 | CF₃ | H | methylsulfonyl | 569 | CF₃ | H | cyclopropylsulfonyl |
| 186 | CF₃ | F | methylsulfonyl | 570 | CF₃ | F | cyclopropylsulfonyl |
| 187 | CF₃ | Br | methylsulfonyl | 571 | CF₃ | Br | cyclopropylsulfonyl |
| 188 | CF₃ | Me | methylsulfonyl | 572 | CF₃ | Me | cyclopropylsulfonyl |
| 189 | CF₃ | iBu | methylsulfonyl | 573 | CF₃ | iBu | cyclopropylsulfonyl |
| 190 | CF₃ | CN | methylsulfonyl | 574 | CF₃ | CN | cyclopropylsulfonyl |
| 191 | CF₃ | OMe | methylsulfonyl | 575 | CF₃ | OMe | cyclopropylsulfonyl |
| 192 | CF₃ | CF₃ | methylsulfonyl | 576 | CF₃ | CF₃ | cyclopropylsulfonyl |
| 193 | H | H | ethylsulfanyl | 577 | H | H | 2,2,2-trifluoroethylsulfanyl |
| 194 | H | F | ethylsulfanyl | 578 | H | F | 2,2,2-trifluoroethylsulfanyl |
| 195 | H | Br | ethylsulfanyl | 579 | H | Br | 2,2,2-trifluoroethylsulfanyl |
| 196 | H | Me | ethylsulfanyl | 580 | H | Me | 2,2,2-trifluoroethylsulfanyl |
| 197 | H | iBu | ethylsulfanyl | 581 | H | iBu | 2,2,2-trifluoroethylsulfanyl |
| 198 | H | CN | ethylsulfanyl | 582 | H | CN | 2,2,2-trifluoroethylsulfanyl |
| 199 | H | OMe | ethylsulfanyl | 583 | H | OMe | 2,2,2-trifluoroethylsulfanyl |
| 200 | H | CF₃ | ethylsulfanyl | 584 | H | CF₃ | 2,2,2-trifluoroethylsulfanyl |
| 201 | F | H | ethylsulfanyl | 585 | F | H | 2,2,2-trifluoroethylsulfanyl |
| 202 | F | F | ethylsulfanyl | 586 | F | F | 2,2,2-trifluoroethylsulfanyl |
| 203 | F | Br | ethylsulfanyl | 587 | F | Br | 2,2,2-trifluoroethylsulfanyl |
| 204 | F | Me | ethylsulfanyl | 588 | F | Me | 2,2,2-trifluoroethylsulfanyl |
| 205 | F | iBu | ethylsulfanyl | 589 | F | iBu | 2,2,2-trifluoroethylsulfanyl |
| 206 | F | CN | ethylsulfanyl | 590 | F | CN | 2,2,2-trifluoroethylsulfanyl |
| 207 | F | OMe | ethylsulfanyl | 591 | F | OMe | 2,2,2-trifluoroethylsulfanyl |
| 208 | F | CF₃ | ethylsulfanyl | 592 | F | CF₃ | 2,2,2-trifluoroethylsulfanyl |
| 209 | Br | H | ethylsulfanyl | 593 | Br | H | 2,2,2-trifluoroethylsulfanyl |
| 210 | Br | F | ethylsulfanyl | 594 | Br | F | 2,2,2-trifluoroethylsulfanyl |
| 211 | Br | Br | ethylsulfanyl | 595 | Br | Br | 2,2,2-trifluoroethylsulfanyl |
| 212 | Br | Me | ethylsulfanyl | 596 | Br | Me | 2,2,2-trifluoroethylsulfanyl |
| 213 | Br | iBu | ethylsulfanyl | 597 | Br | iBu | 2,2,2-trifluoroethylsulfanyl |
| 214 | Br | CN | ethylsulfanyl | 598 | Br | CN | 2,2,2-trifluoroethylsulfanyl |
| 215 | Br | OMe | ethylsulfanyl | 599 | Br | OMe | 2,2,2-trifluoroethylsulfanyl |
| 216 | Br | CF₃ | ethylsulfanyl | 600 | Br | CF₃ | 2,2,2-trifluoroethylsulfanyl |
| 217 | Me | H | ethylsulfanyl | 601 | Me | H | 2,2,2-trifluoroethylsulfanyl |
| 218 | Me | F | ethylsulfanyl | 602 | Me | F | 2,2,2-trifluoroethylsulfanyl |
| 219 | Me | Br | ethylsulfanyl | 603 | Me | Br | 2,2,2-trifluoroethylsulfanyl |
| 220 | Me | Me | ethylsulfanyl | 604 | Me | Me | 2,2,2-trifluoroethylsulfanyl |
| 221 | Me | iBu | ethylsulfanyl | 605 | Me | iBu | 2,2,2-trifluoroethylsulfanyl |
| 222 | Me | CN | ethylsulfanyl | 606 | Me | CN | 2,2,2-trifluoroethylsulfanyl |
| 223 | Me | OMe | ethylsulfanyl | 607 | Me | OMe | 2,2,2-trifluoroethylsulfanyl |
| 224 | Me | CF₃ | ethylsulfanyl | 608 | Me | CF₃ | 2,2,2-trifluoroethylsulfanyl |
| 225 | iBu | H | ethylsulfanyl | 609 | iBu | H | 2,2,2-trifluoroethylsulfanyl |
| 226 | iBu | F | ethylsulfanyl | 610 | iBu | F | 2,2,2-trifluoroethylsulfanyl |
| 227 | iBu | Br | ethylsulfanyl | 611 | iBu | Br | 2,2,2-trifluoroethylsulfanyl |
| 228 | iBu | Me | ethylsulfanyl | 612 | iBu | Me | 2,2,2-trifluoroethylsulfanyl |
| 229 | iBu | iBu | ethylsulfanyl | 613 | iBu | iBu | 2,2,2-trifluoroethylsulfanyl |
| 230 | iBu | CN | ethylsulfanyl | 614 | iBu | CN | 2,2,2-trifluoroethylsulfanyl |
| 231 | iBu | OMe | ethylsulfanyl | 615 | iBu | OMe | 2,2,2-trifluoroethylsulfanyl |
| 232 | iBu | CF₃ | ethylsulfanyl | 616 | iBu | CF₃ | 2,2,2-trifluoroethylsulfanyl |
| 233 | CN | H | ethylsulfanyl | 617 | CN | H | 2,2,2-trifluoroethylsulfanyl |
| 234 | CN | F | ethylsulfanyl | 618 | CN | F | 2,2,2-trifluoroethylsulfanyl |
| 235 | CN | Br | ethylsulfanyl | 619 | CN | Br | 2,2,2-trifluoroethylsulfanyl |
| 236 | CN | Me | ethylsulfanyl | 620 | CN | Me | 2,2,2-trifluoroethylsulfanyl |
| 237 | CN | iBu | ethylsulfanyl | 621 | CN | iBu | 2,2,2-trifluoroethylsulfanyl |
| 238 | CN | CN | ethylsulfanyl | 622 | CN | CN | 2,2,2-trifluoroethylsulfanyl |
| 239 | CN | OMe | ethylsulfanyl | 623 | CN | OMe | 2,2,2-trifluoroethylsulfanyl |
| 240 | CN | CF₃ | ethylsulfanyl | 624 | CN | CF₃ | 2,2,2-trifluoroethylsulfanyl |
| 241 | OMe | H | ethylsulfanyl | 625 | OMe | H | 2,2,2-trifluoroethylsulfanyl |
| 242 | OMe | F | ethylsulfanyl | 626 | OMe | F | 2,2,2-trifluoroethylsulfanyl |
| 243 | OMe | Br | ethylsulfanyl | 627 | OMe | Br | 2,2,2-trifluoroethylsulfanyl |
| 244 | OMe | Me | ethylsulfanyl | 628 | OMe | Me | 2,2,2-trifluoroethylsulfanyl |
| 245 | OMe | iBu | ethylsulfanyl | 629 | OMe | iBu | 2,2,2-trifluoroethylsulfanyl |
| 246 | OMe | CN | ethylsulfanyl | 630 | OMe | CN | 2,2,2-trifluoroethylsulfanyl |
| 247 | OMe | OMe | ethylsulfanyl | 631 | OMe | OMe | 2,2,2-trifluoroethylsulfanyl |
| 248 | OMe | CF₃ | ethylsulfanyl | 632 | OMe | CF₃ | 2,2,2-trifluoroethylsulfanyl |
| 249 | CF₃ | H | ethylsulfanyl | 633 | CF₃ | H | 2,2,2-trifluoroethylsulfanyl |
| 250 | CF₃ | F | ethylsulfanyl | 634 | CF₃ | F | 2,2,2-trifluoroethylsulfanyl |
| 251 | CF₃ | Br | ethylsulfanyl | 635 | CF₃ | Br | 2,2,2-trifluoroethylsulfanyl |
| 252 | CF₃ | Me | ethylsulfanyl | 636 | CF₃ | Me | 2,2,2-trifluoroethylsulfanyl |
| 253 | CF₃ | iBu | ethylsulfanyl | 637 | CF₃ | iBu | 2,2,2-trifluoroethylsulfanyl |
| 254 | CF₃ | CN | ethylsulfanyl | 638 | CF₃ | CN | 2,2,2-trifluoroethylsulfanyl |
| 255 | CF₃ | OMe | ethylsulfanyl | 639 | CF₃ | OMe | 2,2,2-trifluoroethylsulfanyl |
| 256 | CF₃ | CF₃ | ethylsulfanyl | 640 | CF₃ | CF₃ | 2,2,2-trifluoroethylsulfanyl |
| 257 | H | H | ethylsulfinyl | 641 | H | H | 2,2,2-trifluoroethylsulfinyl |
| 258 | H | F | ethylsulfinyl | 642 | H | F | 2,2,2-trifluoroethylsulfinyl |
| 259 | H | Br | ethylsulfinyl | 643 | H | Br | 2,2,2-trifluoroethylsulfinyl |
| 260 | H | Me | ethylsulfinyl | 644 | H | Me | 2,2,2-trifluoroethylsulfinyl |
| 261 | H | iBu | ethylsulfinyl | 645 | H | iBu | 2,2,2-trifluoroethylsulfinyl |
| 262 | H | CN | ethylsulfinyl | 646 | H | CN | 2,2,2-trifluoroethylsulfinyl |
| 263 | H | OMe | ethylsulfinyl | 647 | H | OMe | 2,2,2-trifluoroethylsulfinyl |
| 264 | H | CF₃ | ethylsulfinyl | 648 | H | CF₃ | 2,2,2-trifluoroethylsulfinyl |
| 265 | F | H | ethylsulfinyl | 649 | F | H | 2,2,2-trifluoroethylsulfinyl |
| 266 | F | F | ethylsulfinyl | 650 | F | F | 2,2,2-trifluoroethylsulfinyl |
| 267 | F | Br | ethylsulfinyl | 651 | F | Br | 2,2,2-trifluoroethylsulfinyl |
| 268 | F | Me | ethylsulfinyl | 652 | F | Me | 2,2,2-trifluoroethylsulfinyl |
| 269 | F | iBu | ethylsulfinyl | 653 | F | iBu | 2,2,2-trifluoroethylsulfinyl |
| 270 | F | CN | ethylsulfinyl | 654 | F | CN | 2,2,2-trifluoroethylsulfinyl |
| 271 | F | OMe | ethylsulfinyl | 655 | F | OMe | 2,2,2-trifluoroethylsulfinyl |
| 272 | F | CF₃ | ethylsulfinyl | 656 | F | CF₃ | 2,2,2-trifluoroethylsulfinyl |
| 273 | Br | H | ethylsulfinyl | 657 | Br | H | 2,2,2-trifluoroethylsulfinyl |
| 274 | Br | F | ethylsulfinyl | 658 | Br | F | 2,2,2-trifluoroethylsulfinyl |
| 275 | Br | Br | ethylsulfinyl | 659 | Br | Br | 2,2,2-trifluoroethylsulfinyl |
| 276 | Br | Me | ethylsulfinyl | 660 | Br | Me | 2,2,2-trifluoroethylsulfinyl |
| 277 | Br | iBu | ethylsulfinyl | 661 | Br | iBu | 2,2,2-trifluoroethylsulfinyl |
| 278 | Br | CN | ethylsulfinyl | 662 | Br | CN | 2,2,2-trifluoroethylsulfinyl |
| 279 | Br | OMe | ethylsulfinyl | 663 | Br | OMe | 2,2,2-trifluoroethylsulfinyl |
| 280 | Br | CF₃ | ethylsulfinyl | 664 | Br | CF₃ | 2,2,2-trifluoroethylsulfinyl |
| 281 | Me | H | ethylsulfinyl | 665 | Me | H | 2,2,2-trifluoroethylsulfinyl |
| 282 | Me | F | ethylsulfinyl | 666 | Me | F | 2,2,2-trifluoroethylsulfinyl |
| 283 | Me | Br | ethylsulfinyl | 667 | Me | Br | 2,2,2-trifluoroethylsulfinyl |
| 284 | Me | Me | ethylsulfinyl | 668 | Me | Me | 2,2,2-trifluoroethylsulfinyl |
| 285 | Me | iBu | ethylsulfinyl | 669 | Me | iBu | 2,2,2-trifluoroethylsulfinyl |
| 286 | Me | CN | ethylsulfinyl | 670 | Me | CN | 2,2,2-trifluoroethylsulfinyl |
| 287 | Me | OMe | ethylsulfinyl | 671 | Me | OMe | 2,2,2-trifluoroethylsulfinyl |
| 288 | Me | CF₃ | ethylsulfinyl | 672 | Me | CF₃ | 2,2,2-trifluoroethylsulfinyl |
| 289 | iBu | H | ethylsulfinyl | 673 | iBu | H | 2,2,2-trifluoroethylsulfinyl |
| 290 | iBu | F | ethylsulfinyl | 674 | iBu | F | 2,2,2-trifluoroethylsulfinyl |
| 291 | iBu | Br | ethylsulfinyl | 675 | iBu | Br | 2,2,2-trifluoroethylsulfinyl |
| 292 | iBu | Me | ethylsulfinyl | 676 | iBu | Me | 2,2,2-trifluoroethylsulfinyl |
| 293 | iBu | iBu | ethylsulfinyl | 677 | iBu | iBu | 2,2,2-trifluoroethylsulfinyl |
| 294 | iBu | CN | ethylsulfinyl | 678 | iBu | CN | 2,2,2-trifluoroethylsulfinyl |
| 295 | iBu | OMe | ethylsulfinyl | 679 | iBu | OMe | 2,2,2-trifluoroethylsulfinyl |
| 296 | iBu | CF₃ | ethylsulfinyl | 680 | iBu | CF₃ | 2,2,2-trifluoroethylsulfinyl |
| 297 | CN | H | ethylsulfinyl | 681 | CN | H | 2,2,2-trifluoroethylsulfinyl |
| 298 | CN | F | ethylsulfinyl | 682 | CN | F | 2,2,2-trifluoroethylsulfinyl |
| 299 | CN | Br | ethylsulfinyl | 683 | CN | Br | 2,2,2-trifluoroethylsulfinyl |
| 300 | CN | Me | ethylsulfinyl | 684 | CN | Me | 2,2,2-trifluoroethylsulfinyl |
| 301 | CN | iBu | ethylsulfinyl | 685 | CN | iBu | 2,2,2-trifluoroethylsulfinyl |
| 302 | CN | CN | ethylsulfinyl | 686 | CN | CN | 2,2,2-trifluoroethylsulfinyl |
| 303 | CN | OMe | ethylsulfinyl | 687 | CN | OMe | 2,2,2-trifluoroethylsulfinyl |
| 304 | CN | CF₃ | ethylsulfinyl | 688 | CN | CF₃ | 2,2,2-trifluoroethylsulfinyl |
| 305 | OMe | H | ethylsulfinyl | 689 | OMe | H | 2,2,2-trifluoroethylsulfinyl |
| 306 | OMe | F | ethylsulfinyl | 690 | OMe | F | 2,2,2-trifluoroethylsulfinyl |
| 307 | OMe | Br | ethylsulfinyl | 691 | OMe | Br | 2,2,2-trifluoroethylsulfinyl |
| 308 | OMe | Me | ethylsulfinyl | 692 | OMe | Me | 2,2,2-trifluoroethylsulfinyl |
| 309 | OMe | iBu | ethylsulfinyl | 693 | OMe | iBu | 2,2,2-trifluoroethylsulfinyl |
| 310 | OMe | CN | ethylsulfinyl | 694 | OMe | CN | 2,2,2-trifluoroethylsulfinyl |
| 311 | OMe | OMe | ethylsulfinyl | 695 | OMe | OMe | 2,2,2-trifluoroethylsulfinyl |
| 312 | OMe | CF₃ | ethylsulfinyl | 696 | OMe | CF₃ | 2,2,2-trifluoroethylsulfinyl |
| 313 | CF₃ | H | ethylsulfinyl | 697 | CF₃ | H | 2,2,2-trifluoroethylsulfinyl |
| 314 | CF₃ | F | ethylsulfinyl | 698 | CF₃ | F | 2,2,2-trifluoroethylsulfinyl |
| 315 | CF₃ | Br | ethylsulfinyl | 699 | CF₃ | Br | 2,2,2-trifluoroethylsulfinyl |
| 316 | CF₃ | Me | ethylsulfinyl | 700 | CF₃ | Me | 2,2,2-trifluoroethylsulfinyl |
| 317 | CF₃ | iBu | ethylsulfinyl | 701 | CF₃ | iBu | 2,2,2-trifluoroethylsulfinyl |
| 318 | CF₃ | CN | ethylsulfinyl | 702 | CF₃ | CN | 2,2,2-trifluoroethylsulfinyl |
| 319 | CF₃ | OMe | ethylsulfinyl | 703 | CF₃ | OMe | 2,2,2-trifluoroethylsulfinyl |
| 320 | CF₃ | CF₃ | ethylsulfinyl | 704 | CF₃ | CF₃ | 2,2,2-trifluoroethylsulfinyl |
| 321 | H | H | ethylsulfonyl | 705 | H | H | 2,2,2-trifluoroethylsulfonyl |
| 322 | H | F | ethylsulfonyl | 706 | H | F | 2,2,2-trifluoroethylsulfonyl |
| 323 | H | Br | ethylsulfonyl | 707 | H | Br | 2,2,2-trifluoroethylsulfonyl |
| 324 | H | Me | ethylsulfonyl | 708 | H | Me | 2,2,2-trifluoroethylsulfonyl |
| 325 | H | iBu | ethylsulfonyl | 709 | H | iBu | 2,2,2-trifluoroethylsulfonyl |
| 326 | H | CN | ethylsulfonyl | 710 | H | CN | 2,2,2-trifluoroethylsulfonyl |
| 327 | H | OMe | ethylsulfonyl | 711 | H | OMe | 2,2,2-trifluoroethylsulfonyl |
| 328 | H | CF₃ | ethylsulfonyl | 712 | H | CF₃ | 2,2,2-trifluoroethylsulfonyl |
| 329 | F | H | ethylsulfonyl | 713 | F | H | 2,2,2-trifluoroethylsulfonyl |
| 330 | F | F | ethylsulfonyl | 714 | F | F | 2,2,2-trifluoroethylsulfonyl |
| 331 | F | Br | ethylsulfonyl | 715 | F | Br | 2,2,2-trifluoroethylsulfonyl |
| 332 | F | Me | ethylsulfonyl | 716 | F | Me | 2,2,2-trifluoroethylsulfonyl |
| 333 | F | iBu | ethylsulfonyl | 717 | F | iBu | 2,2,2-trifluoroethylsulfonyl |
| 334 | F | CN | ethylsulfonyl | 718 | F | CN | 2,2,2-trifluoroethylsulfonyl |
| 335 | F | OMe | ethylsulfonyl | 719 | F | OMe | 2,2,2-trifluoroethylsulfonyl |
| 336 | F | CF₃ | ethylsulfonyl | 720 | F | CF₃ | 2,2,2-trifluoroethylsulfonyl |
| 337 | Br | H | ethylsulfonyl | 721 | Br | H | 2,2,2-trifluoroethylsulfonyl |
| 338 | Br | F | ethylsulfonyl | 722 | Br | F | 2,2,2-trifluoroethylsulfonyl |
| 339 | Br | Br | ethylsulfonyl | 723 | Br | Br | 2,2,2-trifluoroethylsulfonyl |
| 340 | Br | Me | ethylsulfonyl | 724 | Br | Me | 2,2,2-trifluoroethylsulfonyl |
| 341 | Br | iBu | ethylsulfonyl | 725 | Br | iBu | 2,2,2-trifluoroethylsulfonyl |
| 342 | Br | CN | ethylsulfonyl | 726 | Br | CN | 2,2,2-trifluoroethylsulfonyl |
| 343 | Br | OMe | ethylsulfonyl | 727 | Br | OMe | 2,2,2-trifluoroethylsulfonyl |
| 344 | Br | CF₃ | ethylsulfonyl | 728 | Br | CF₃ | 2,2,2-trifluoroethylsulfonyl |
| 345 | Me | H | ethylsulfonyl | 729 | Me | H | 2,2,2-trifluoroethylsulfonyl |
| 346 | Me | F | ethylsulfonyl | 730 | Me | F | 2,2,2-trifluoroethylsulfonyl |
| 347 | Me | Br | ethylsulfonyl | 731 | Me | Br | 2,2,2-trifluoroethylsulfonyl |
| 348 | Me | Me | ethylsulfonyl | 732 | Me | Me | 2,2,2-trifluoroethylsulfonyl |
| 349 | Me | iBu | ethylsulfonyl | 733 | Me | iBu | 2,2,2-trifluoroethylsulfonyl |
| 350 | Me | CN | ethylsulfonyl | 734 | Me | CN | 2,2,2-trifluoroethylsulfonyl |
| 351 | Me | OMe | ethylsulfonyl | 735 | Me | OMe | 2,2,2-trifluoroethylsulfonyl |
| 352 | Me | CF₃ | ethylsulfonyl | 736 | Me | CF₃ | 2,2,2-trifluoroethylsulfonyl |
| 353 | iBu | H | ethylsulfonyl | 737 | iBu | H | 2,2,2-trifluoroethylsulfonyl |
| 354 | iBu | F | ethylsulfonyl | 738 | iBu | F | 2,2,2-trifluoroethylsulfonyl |
| 355 | iBu | Br | ethylsulfonyl | 739 | iBu | Br | 2,2,2-trifluoroethylsulfonyl |
| 356 | iBu | Me | ethylsulfonyl | 740 | iBu | Me | 2,2,2-trifluoroethylsulfonyl |
| 357 | iBu | iBu | ethylsulfonyl | 741 | iBu | iBu | 2,2,2-trifluoroethylsulfonyl |
| 358 | iBu | CN | ethylsulfonyl | 742 | iBu | CN | 2,2,2-trifluoroethylsulfonyl |
| 359 | iBu | OMe | ethylsulfonyl | 743 | iBu | OMe | 2,2,2-trifluoroethylsulfonyl |
| 360 | iBu | CF₃ | ethylsulfonyl | 744 | iBu | CF₃ | 2,2,2-trifluoroethylsulfonyl |
| 361 | CN | H | ethylsulfonyl | 745 | CN | H | 2,2,2-trifluoroethylsulfonyl |
| 362 | CN | F | ethylsulfonyl | 746 | CN | F | 2,2,2-trifluoroethylsulfonyl |
| 363 | CN | Br | ethylsulfonyl | 747 | CN | Br | 2,2,2-trifluoroethylsulfonyl |
| 364 | CN | Me | ethylsulfonyl | 748 | CN | Me | 2,2,2-trifluoroethylsulfonyl |
| 365 | CN | iBu | ethylsulfonyl | 749 | CN | iBu | 2,2,2-trifluoroethylsulfonyl |
| 366 | CN | CN | ethylsulfonyl | 750 | CN | CN | 2,2,2-trifluoroethylsulfonyl |
| 367 | CN | OMe | ethylsulfonyl | 751 | CN | OMe | 2,2,2-trifluoroethylsulfonyl |
| 368 | CN | CF₃ | ethylsulfonyl | 752 | CN | CF₃ | 2,2,2-trifluoroethylsulfonyl |
| 369 | OMe | H | ethylsulfonyl | 753 | OMe | H | 2,2,2-trifluoroethylsulfonyl |
| 370 | OMe | F | ethylsulfonyl | 754 | OMe | F | 2,2,2-trifluoroethylsulfonyl |
| 371 | OMe | Br | ethylsulfonyl | 755 | OMe | Br | 2,2,2-trifluoroethylsulfonyl |
| 372 | OMe | Me | ethylsulfonyl | 756 | OMe | Me | 2,2,2-trifluoroethylsulfonyl |
| 373 | OMe | iBu | ethylsulfonyl | 757 | OMe | iBu | 2,2,2-trifluoroethylsulfonyl |
| 374 | OMe | CN | ethylsulfonyl | 758 | OMe | CN | 2,2,2-trifluoroethylsulfonyl |
| 375 | OMe | OMe | ethylsulfonyl | 759 | OMe | OMe | 2,2,2-trifluoroethylsulfonyl |
| 376 | OMe | CF₃ | ethylsulfonyl | 760 | OMe | CF₃ | 2,2,2-trifluoroethylsulfonyl |
| 377 | CF₃ | H | ethylsulfonyl | 761 | CF₃ | H | 2,2,2-trifluoroethylsulfonyl |
| 378 | CF₃ | F | ethylsulfonyl | 762 | CF₃ | F | 2,2,2-trifluoroethylsulfonyl |
| 379 | CF₃ | Br | ethylsulfonyl | 763 | CF₃ | Br | 2,2,2-trifluoroethylsulfonyl |
| 380 | CF₃ | Me | ethylsulfonyl | 764 | CF₃ | Me | 2,2,2-trifluoroethylsulfonyl |
| 381 | CF₃ | iBu | ethylsulfonyl | 765 | CF₃ | iBu | 2,2,2-trifluoroethylsulfonyl |
| 382 | CF₃ | CN | ethylsulfonyl | 766 | CF₃ | CN | 2,2,2-trifluoroethylsulfonyl |
| 383 | CF₃ | OMe | ethylsulfonyl | 767 | CF₃ | OMe | 2,2,2-trifluoroethylsulfonyl |
| 384 | CF₃ | CF₃ | ethylsulfonyl | 768 | CF₃ | CF₃ | 2,2,2-trifluoroethylsulfonyl |

Table A-1 provides 768 compounds A-1.001 to A.1.768 of Formula (I) wherein R¹ is 4-(trifluoromethoxy)phenyl, R³ is hydrogen, R⁶ is hydrogen, X is oxygen, and R², R⁴, and R⁵ are as defined in Table 1.

Table A-2 provides 768 compounds A-2.001 to A.2.768 of Formula (I) wherein R¹ is 4-(trifluoromethoxy)phenyl, R³ is methyl, R⁶ is hydrogen, X is oxygen, and R², R⁴, and R⁵ are as defined in Table 1.

Table A-3 provides 768 compounds A-3.001 to A.3.768 of Formula (I) wherein R¹ is 4-(trifluoromethoxy)phenyl, R³ is ethyl, R⁶ is hydrogen, X is oxygen, and R², R⁴, and R⁵ are as defined in Table 1.

Table A-4 provides 768 compounds A-4.001 to A.4.768 of Formula (I) wherein R¹ is 4-chlorophenyl, R³ is hydrogen, R⁶ is hydrogen, X is oxygen, and R², R⁴, and R⁵ are as defined in Table 1.

Table A-5 provides 768 compounds A-5.001 to A.5.768 of Formula (I) wherein R¹ is 4-chlorophenyl, R³ is methyl, R⁶ is hydrogen, X is oxygen, and R², R⁴, and R⁵ are as defined in Table 1.

Table A-6 provides 768 compounds A-6.001 to A.6.768 of Formula (I) wherein R¹ is 4-chlorophenyl, R³ is ethyl, R⁶ is hydrogen, X is oxygen, and R², R⁴, and R⁵ are as defined in Table 1.

### Formulation Examples

| Wettable powders | a) | b) | c) |
|---|---|---|---|
| active ingredient [compound of formula (I)] | 25% | 50% | 75% |
| sodium lignosulfonate | 5% | 5% | — |
| sodium lauryl sulfate | 3% | — | 5% |
| sodium diisobutylnaphthalenesulfonate | — | 6% | 10% |
| phenol polyethylene glycol ether | — | 2% | — |
| (7-8 mol of ethylene oxide) | | | |
| highly dispersed silicic acid | 5% | 10% | 10% |
| Kaolin | 62% | 27% | — |

The active ingredient is thoroughly mixed with the adjuvants and the mixture is thoroughly ground in a suitable mill, affording wettable powders that can be diluted with water to give suspensions of the desired concentration.

| Powders for dry seed treatment | a) | b) | c) |
|---|---|---|---|
| active ingredient [compound of formula (I)] | 25% | 50% | 75% |
| light mineral oil | 5% | 5% | 5% |
| highly dispersed silicic acid | 5% | 5% | — |
| Kaolin | 65% | 40% | — |
| Talcum | — | | 20% |

The active ingredient is thoroughly mixed with the adjuvants and the mixture is thoroughly ground in a suitable mill, affording powders that can be used directly for seed treatment.

### Emulsifiable Concentrate

| | |
|---|---|
| active ingredient [compound of formula (I)] | 10% |
| octylphenol polyethylene glycol ether | 3% |
| (4-5 mol of ethylene oxide) | |
| calcium dodecylbenzenesulfonate | 3% |
| castor oil polyglycol ether (35 mol of ethylene oxide) | 4% |
| Cyclohexanone | 30% |
| xylene mixture | 50% |

Emulsions of any required dilution, which can be used in plant protection, can be obtained from this concentrate by dilution with water.

| Dusts | a) | b) | c) |
|---|---|---|---|
| Active ingredient [compound of formula (I)] | 5% | 6% | 4% |
| talcum | 95% | — | — |
| Kaolin | — | 94% | — |
| mineral filler | — | — | 96% |

Ready-for-use dusts are obtained by mixing the active ingredient with the carrier and grinding the mixture in a suitable mill. Such powders can also be used for dry dressings for seed.

### Extruder Granules

| | |
|---|---|
| Active ingredient [compound of formula (I)] | 15% |
| sodium lignosulfonate | 2% |
| carboxymethylcellulose | 1% |
| Kaolin | 82% |

The active ingredient is mixed and ground with the adjuvants, and the mixture is moistened with water.
The mixture is extruded and then dried in a stream of air.

### Coated Granules

| | |
|---|---|
| Active ingredient [compound of formula (I)] | 8% |
| polyethylene glycol (mol. wt. 200) | 3% |
| Kaolin | 89% |

The finely ground active ingredient is uniformly applied, in a mixer, to the kaolin moistened with polyethylene glycol. Non-dusty coated granules are obtained in this manner.

### Suspension Concentrate

| | |
|---|---|
| active ingredient [compound of formula (I)] | 40% |
| propylene glycol | 10% |
| nonylphenol polyethylene glycol ether (15 mol of ethylene oxide) | 6% |
| Sodium lignosulfonate | 10% |
| carboxymethylcellulose | 1% |
| silicone oil (in the form of a 75% emulsion in water) | 1% |
| Water | 32% |

The finely ground active ingredient is intimately mixed with the adjuvants, giving a suspension concentrate from which suspensions of any desired dilution can be obtained by dilution with water. Using such dilutions, living plants as well as plant propagation material can be treated and protected against infestation by microorganisms, by spraying, pouring or immersion.

### Flowable Concentrate for Seed Treatment

| | |
|---|---|
| active ingredient [compound of formula (I)] | 40% |
| propylene glycol | 5% |
| copolymer butanol PO/EO | 2% |
| tristyrenephenole with 10-20 moles EO | 2% |
| 1,2-benzisothiazolin-3-one (in the form of a 20% solution in | 0.5% |
| water) | |
| monoazo-pigment calcium salt | 5% |
| Silicone oil (in the form of a 75% emulsion in water) | 0.2% |
| Water | 45.3% |

The finely ground active ingredient is intimately mixed with the adjuvants, giving a suspension concentrate from which suspensions of any desired dilution can be obtained by dilution with water. Using such dilutions, living plants as well as plant propagation material can be treated and protected against infestation by microorganisms, by spraying, pouring or immersion.

### Slow Release Capsule Suspension

28 parts of a combination of the compound of formula (I) are mixed with 2 parts of an aromatic solvent and 7 parts of toluene diisocyanate/polymethylene-polyphenylisocyanate-mixture (8:1). This mixture is emulsified in a mixture of 1.2 parts of polyvinyl alcohol, 0.05 parts of a defoamer and 51.6 parts of water until the desired particle size is achieved. To this emulsion a mixture of 2.8 parts 1,6-diaminohexane in 5.3 parts of water is added. The mixture is agitated until the polymerization reaction is completed. The obtained capsule suspension is stabilized by adding 0.25 parts of a thickener and 3 parts of a dispersing agent. The capsule suspension formulation contains 28% of the active ingredients. The medium capsule diameter is 8-15 microns. The resulting formulation is applied to seeds as an aqueous suspension in an apparatus suitable for that purpose.

### EXAMPLES

The following non-limiting examples provide specific synthesis methods for representative compounds of the present invention, as referred to in Table 2 below.

### Example 1: Synthesis of 5-methylsulfonyl-4-oxo-1-[4-(trifluoromethoxy)phenyl]cinnoline-3-carboxylic acid (Compound P2)

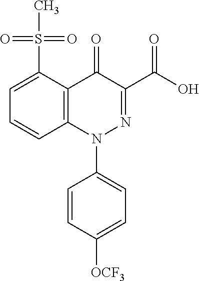 [SMILES: CS(=O)(=O)c1cccc2c1c(=O)c(C(=O)O)nn2-c1ccc(OC(F)(F)F)cc1]

Step 1: Synthesis of ethyl 3-(2,6-difluorophenyl)-3-oxo-propanoate

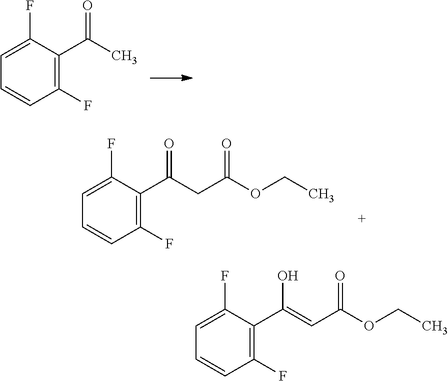

To a solution of potassium;3-ethoxy-3-oxo-propanoic acid (6.11 g, 35.7 mmol) in acetonitrile (66 mL) at 0° C. and under nitrogen was added triethylamine (3.78 g, 37.4 mmol) and dichloromagnesium (4.1 g, 42.5 mmol). The reaction mixture was stirred at room temperature for 3.5 hours. The reaction mixture was cooled to 0° C. and 2,6-difluorobenzoyl chloride (3.0 g, 17 mmol) was added portionwise. The reaction mixture was stirred for 1.5 hours in ice and then at room temperature for 2 hours before standing for 18 hours. The reaction mixture was evaporated under reduced pressure and azeotroped with toluene. The residue was suspended in ethyl acetate (50 mL) and 2M aqueous hydrochloric acid. The phases were separated and the aqueous was re-extracted twice with ethyl acetate. The combined organic extracts were dried over magnesium sulfate and evaporated to dryness under reduced pressure to give the crude desired product (mixture of tautomers) as a pale-yellow liquid (4.5 g, 20 mmol). 1 H NMR (400 MHz, chloroform) 6=7.53-7.37 (m, 1H), 7.04-6.88 (m, 2H), 4.30-4.22 (m, 2H), 3.47-3.38 (m, 2H), 1.34-1.28 (m, 3H) (data for keto form only).

Step 2: Synthesis of ethyl (2E)-3-(2,6-difluorophenyl)-3-oxo-2-[[4-(trifluoromethoxy)phenyl]hydrazono]propanoate

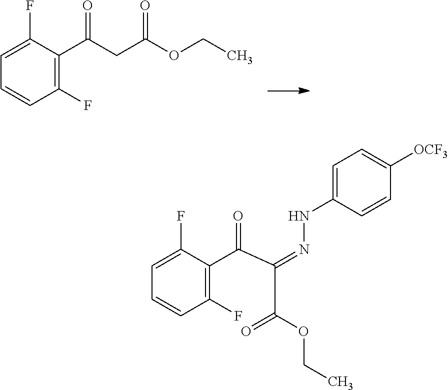 [SMILES: CCOC(=O)CC(=O)c1c(F)cccc1F ; CCOC(=O)/C(=N/Nc1ccc(OC(F)(F)F)cc1)C(=O)c1c(F)cccc1F]

To a solution of 4-(trifluoromethoxy)aniline (1.10 g, 6.25 mmol) in hydrochloric acid (5.2 mL, 31 mmol) at 0° C. was added a solution of sodium nitrite (0.48 g, 6.87 mmol) in water (1.3 mL). The reaction mixture was stirred for 30 mins at 0° C. before being added portionwise to a suspension of ethyl 3-(2,6-difluorophenyl)-3-oxo-propanoate (2.03 g, 6.25 mmol) and potassium acetate (3.1 g, 31.2 mmol) in water (1.2 mL). The reaction mixture was stirred for 2.75 hours before the solution was decanted to leave a red gum. This was dissolved in ethyl acetate, dried over magnesium sulfate, and evaporated to dryness under reduced pressure to give the desired product as a red solid (2.6 g, 6.25 mmol, 64%). ¹H NMR (400 MHz, chloroform) 6=7.45-7.41 (m, 3H), 7.18-7.11 (m, 2H), 7.05-7.00 (m, 2H), 4.49-4.35 (m, 2H), 1.50-1.35 (m, 3H)

Step 3: Synthesis of ethyl 5-fluoro-4-oxo-1-[4-(trifluoromethoxy)phenyl]cinnoline-3-carboxylate

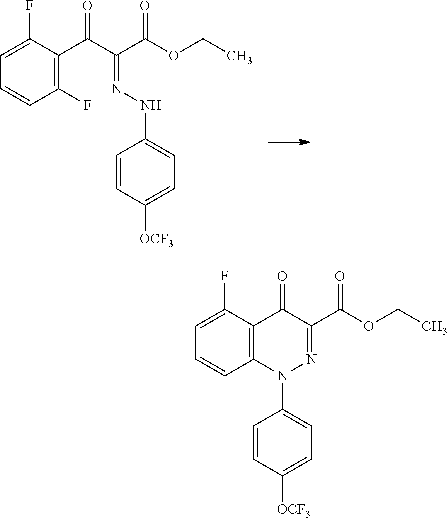 [SMILES: CCOC(=O)/C(=N\Nc1ccc(OC(F)(F)F)cc1)C(=O)c1c(F)cccc1F ; CCOC(=O)c1nn(-c2ccc(OC(F)(F)F)cc2)c2cccc(F)c2c1=O]

To a solution of ethyl (2Z)-3-(2,6-difluorophenyl)-3-oxo-2-[[4-(trifluoromethoxy)phenyl]hydrazono] propanoate (2.16 g, 5.179 mmol) in N,N-dimethylformamide (10 mL) was added potassium carbonate (0.58 g, 5.697 mmol). The reaction was mixture heated at 100° C. for 3.5 hours. The cooled reaction mixture was diluted with water and extracted twice into diethyl ether. The combined organic extracts were dried over magnesium sulfate and evaporated to dryness under reduced pressure to give a red solid. Trituration with cyclohexane gave the desired product as an off-white solid (1.2 g, 3.02 mmol, 58%). ¹H NMR (500 MHz, chloroform) 6=7.61-7.54 (m, 3H), 7.50-7.38 (m, 2H), 7.15-7.04 (m, 1H), 6.97-6.88 (m, 1H), 4.50-4.30 (m, 2H), 1.43-1.33 (m, 3H)

Step 4: Synthesis of 5-fluoro-4-oxo-1-[4-(trifluoromethoxy)phenyl]cinnoline-3-carboxylic acid

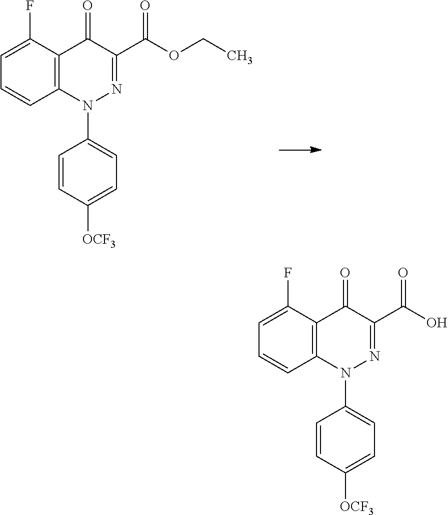 [SMILES: CCOC(=O)c1nn(-c2ccc(OC(F)(F)F)cc2)c2cccc(F)c2c1=O ; O=C(O)c1nn(-c2ccc(OC(F)(F)F)cc2)c2cccc(F)c2c1=O]

To a solution of ethyl 5-fluoro-4-oxo-1-[4-(trifluoromethoxy)phenyl]cinnoline-3-carboxylate (0.71 g, 1.8 mmol) in tetrahydrofuran (10 mL) was added a solution of lithium;hydroxide;hydrate (0.31 g, 7.19 mmol) in water (1.8 mL). The reaction mixture was stirred at room temperature for 2 hours. The reaction mixture was acidified by the addition of 2M aqueous hydrochloric acid and the precipitated solid was collected by filtration and air-dried to give the desired product as an off-white powder (0.65 g, 1.76 mmol, 98%). ¹H NMR (400 MHz, chloroform) 6=7.82-7.73 (m, 1H), 7.63-7.56 (m, 2H), 7.52-7.46 (m, 2H), 7.36-7.30 (m, 1H), 7.17-7.12 (m, 1H)

Step 5: Synthesis of 5-methylsulfonyl-4-oxo-1-[4-(trifluoromethoxy)phenyl]cinnoline-3-carboxylic acid

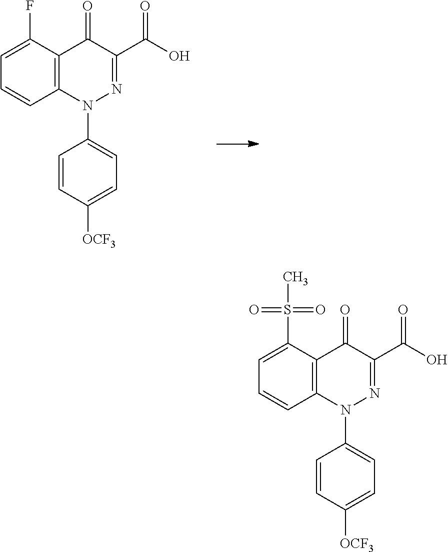 [SMILES: O=C(O)c1nn(-c2ccc(OC(F)(F)F)cc2)c2cccc(F)c2c1=O ; CS(=O)(=O)c1cccc2c1c(=O)c(C(=O)O)nn2-c1ccc(OC(F)(F)F)cc1]

To a solution of 5-fluoro-4-oxo-1-[4-(trifluoromethoxy)phenyl]cinnoline-3-carboxylic acid (0.40 g, 1.1 mmol) in N,N-dimethylformamide (5 mL) was added sodium methanesulfinate (0.34 g, 3.26 mmol). The reaction mixture was heated at 80° C. for 5 hours. The cooled reaction mixture was poured onto ice upon which a yellow solid crashed out of solution. The solid was collected by filtration to give the desired product as a pale-yellow powder (0.37 g, 0.85 mmol, 78%). ¹H NMR (400 MHz, DMSO-d₆) δ=8.34-8.25 (m, 1H), 8.00-7.90 (m, 1H), 7.88-7.83 (m, 2H), 7.75-7.66 (m, 2H), 7.59-7.52 (m, 1H), 3.72-3.63 (m, 3H)

### Example 2: Synthesis of 5-methylsulfanyl-4-oxo-1-[4-(trifluoromethoxy)phenyl]cinnoline-3-carboxylic acid (Compound P1)

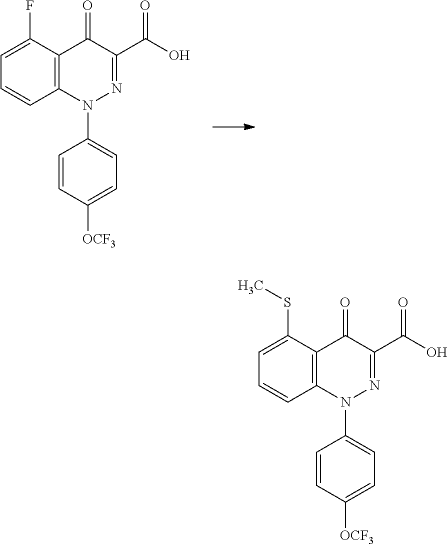 [SMILES: O=C(O)c1nn(-c2ccc(OC(F)(F)F)cc2)c2cccc(F)c2c1=O ; CSc1cccc2c1c(=O)c(C(=O)O)nn2-c1ccc(OC(F)(F)F)cc1]

To a solution of 5-fluoro-4-oxo-1-[4-(trifluoromethoxy)phenyl]cinnoline-3-carboxylic acid (0.20 g, 0.53 mmol) in N,N-dimethylformamide (2 mL) at room temperature was added sodium thiomethoxide (0.11 g, 1.6 mmol). The reaction mixture was heated under microwave irradiation at 100° C. for 1 hour. The reaction mixture was diluted with 2M aqueous hydrochloric acid and the precipitated solid was collected by filtration and washed with water to give the desired product as a yellow powder (0.16 g, 0.40 mmol, 75%). ¹H NMR (400 MHz, DMSO-d₆) δ=7.88-7.78 (m, 2H), 7.75-7.59 (m, 3H), 7.37-7.31 (m, 1H), 6.87-6.81 (m, 1H), 2.49-2.43 (m, 3H)

### Example 3: Synthesis of methyl 5-methylsulfonyl-4-oxo-1-[4-(trifluoromethoxy)phenyl]cinnoline-3-carboxylate (Compound P3)

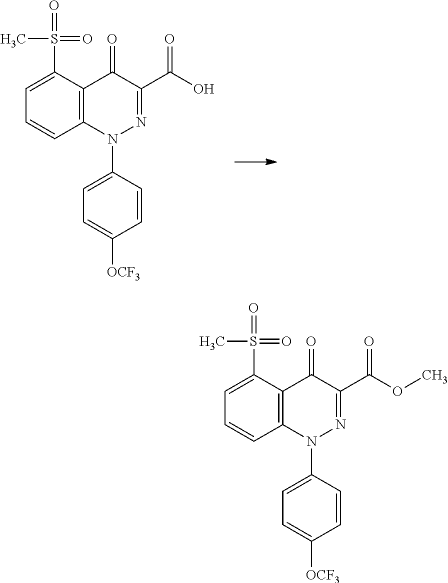

To a suspension of 5-methylsulfonyl-4-oxo-1-[4-(trifluoromethoxy)phenyl]cinnoline-3-carboxylic acid (0.20 g, 0.47 mmol) in methanol (10 mL) was added concentrated sulfuric acid (0.003 mL, 0.047 mmol). The reaction mixture was heated at 80° C. for 2 hours. On cooling, a pale solid precipitated out of solution. The solid was collected by filtration, washed with water, and air-dried to give the desired product as an off-white powder (0.18 g, 0.40 mmol, 87%). ¹H NMR (400 MHz, chloroform) 6=8.49-8.38 (m, 1H), 7.83-7.71 (m, 1H), 7.58-7.53 (m, 2H), 7.51-7.47 (m, 3H), 4.00-3.95 (m, 3H), 3.77-3.66 (m, 3H)

### Example 4: Synthesis of 1-(4-chlorophenyl)-5-methylsulfonyl-4-oxo-cinnoline-3-carboxylic acid (Compound P5)

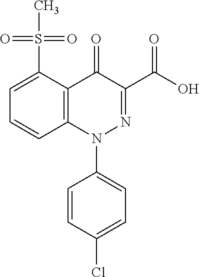 [SMILES: CS(=O)(=O)c1cccc2c1c(=O)c(C(=O)O)nn2-c1ccc(Cl)cc1]

Step 1: Synthesis of ethyl (2E)-2-[(4-chlorophenyl)hydrazono]-3-(2,6-difluorophenyl)-3-oxo-propanoate

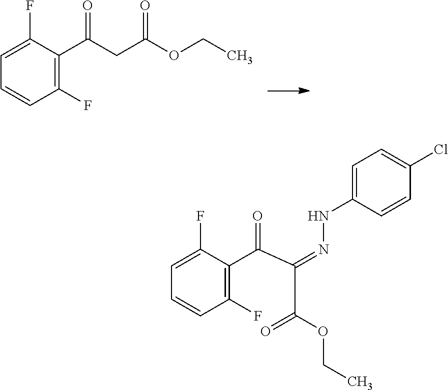 [SMILES: CCOC(=O)CC(=O)c1c(F)cccc1F ; CCOC(=O)/C(=N/Nc1ccc(Cl)cc1)C(=O)c1c(F)cccc1F]

Prepared as for ethyl (2E)-3-(2,6-difluorophenyl)-3-oxo-2-[[4-(trifluoromethoxy)phenyl]hydrazono] propanoate (example 1; step 2) using 4-chloroaniline (1.17 g, 9.2 mmol). After a reaction time of 2.75 hours, the solid was collected by filtration to give the desired product as a yellow solid (2.2 g, 5.9 mmol, 64%). ¹H NMR (400 MHz, chloroform) 6=13.15-13.05 (m, 1H), 7.44-7.32 (m, 1H), 7.27-7.23 (m, 3H), 7.00-6.91 (m, 3H), 4.49-4.38 (m, 2H), 1.51-1.39 (m, 3H)

Step 2: Synthesis of ethyl 1-(4-chlorophenyl)-5-fluoro-4-oxo-cinnoline-3-carboxylate

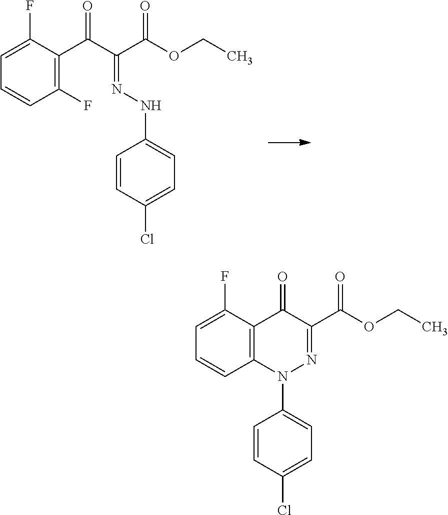 [SMILES: CCOC(=O)/C(=N\Nc1ccc(Cl)cc1)C(=O)c1c(F)cccc1F ; CCOC(=O)c1nn(-c2ccc(Cl)cc2)c2cccc(F)c2c1=O]

Prepared as for ethyl 5-fluoro-4-oxo-1-[4-(trifluoromethoxy)phenyl]cinnoline-3-carboxylate (example 1; step 3) using ethyl (2Z)-2-[(4-chlorophenyl)hydrazono]-3-(2,6-difluorophenyl)-3-oxo-propanoate (2.2 g, 5.9 mmol). On completion of reaction, the cooled reaction mixture was poured onto ice and the precipitated solid was collected by filtration to give the desired product as a yellow powder (1.8 g, 5.3 mmol, 89%). ¹H NMR (400 MHz, chloroform) 6=7.60-7.52 (m, 3H), 7.48-7.40 (m, 2H), 7.14-7.07 (m, 1H), 7.00-6.87 (m, 1H), 4.51-4.40 (m, 2H), 1.45-1.34 (m, 3H)

Step 3: Synthesis of 1-(4-chlorophenyl)-5-fluoro-4-oxo-cinnoline-3-carboxylic acid

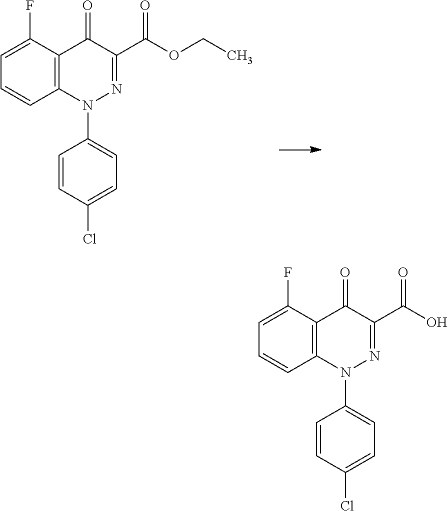 [SMILES: CCOC(=O)c1nn(-c2ccc(Cl)cc2)c2cccc(F)c2c1=O ; O=C(O)c1nn(-c2ccc(Cl)cc2)c2cccc(F)c2c1=O]

Prepared as for 5-fluoro-4-oxo-1-[4-(trifluoromethoxy)phenyl]cinnoline-3-carboxylic acid (example 1; step 4) using ethyl 1-(4-chlorophenyl)-5-fluoro-4-oxo-cinnoline-3-carboxylate (1.29 g, 3.7 mmol) to give the desired product as an off-white solid (1.15 g, 3.6 mmol, 97%). ¹H NMR (500 MHz, chloroform) 6=14.22-13.90 (m, 1H), 7.82-7.74 (m, 1H), 7.63-7.59 (m, 2H), 7.51-7.37 (m, 2H), 7.36-7.26 (m, 1H), 7.19-7.00 (m, 1H)

Step 4: Synthesis of 1-(4-chlorophenyl)-5-methylsulfonyl-4-oxo-cinnoline-3-carboxylic acid

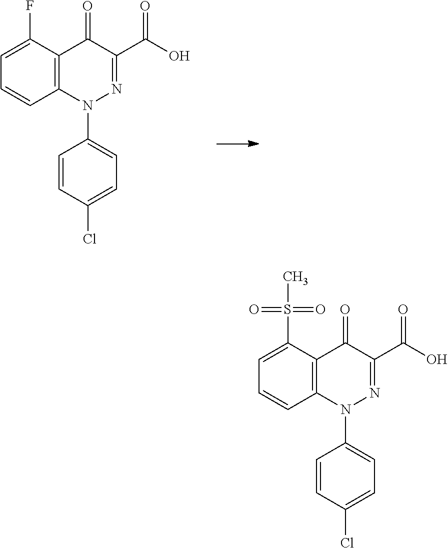 [SMILES: O=C(O)c1nn(-c2ccc(Cl)cc2)c2cccc(F)c2c1=O ; CS(=O)(=O)c1cccc2c1c(=O)c(C(=O)O)nn2-c1ccc(Cl)cc1]

To a solution of 1-(4-chlorophenyl)-5-fluoro-4-oxo-cinnoline-3-carboxylic acid (0.20 g, 0.63 mmol) in N,N-dimethylformamide (2 mL) was added sodium methanesulfinate (0.19 g, 1.9 mmol). The reaction mixture was heated under microwave irradiation at 80° C. for 45+45 minutes. The cooled reaction mixture was poured onto ice and the precipitated solid was collected by filtration to give a pale-yellow powder which was triturated with dichloromethane. Addition of dimethyl sulfoxide/methanol mixture (9:1) resulted in precipitation of a white solid which was collected by filtration to give the desired product as a white solid (0.071 g, 0.19 mmol, 30%). ¹H NMR (400 MHz, DMSO-d₆) δ=8.36-8.27 (m, 1H), 7.99-7.91 (m, 1H), 7.82-7.68 (m, 4H), 7.61-7.46 (m, 1H), 3.73-3.65 (m, 3H)

### Example 5: Synthesis of 1-(4-chlorophenyl)-5-methylsulfanyl-4-oxo-cinnoline-3-carboxylic acid

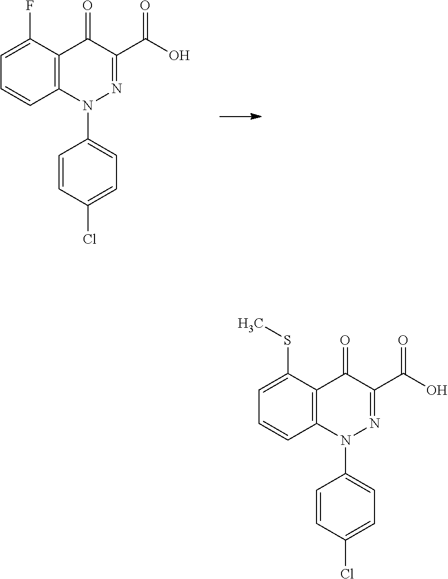 [SMILES: O=C(O)c1nn(-c2ccc(Cl)cc2)c2cccc(F)c2c1=O ; CSc1cccc2c1c(=O)c(C(=O)O)nn2-c1ccc(Cl)cc1]

A solution of 1-(4-chlorophenyl)-5-fluoro-4-oxo-cinnoline-3-carboxylic acid (0.20 g, 0.63 mmol) and sodium thiomethoxide (0.13 g, 1.9 mmol) in N,N-dimethylformamide (2 mL) was heated under microwave irradiation at 80° C. for 60+60 minutes. The cooled reaction mixture was diluted with 2M aqueous hydrochloric acid resulting in precipitation of a yellow solid which was insoluble upon extraction into either ethyl acetate or dichloromethane. The solids were collected by filtration from the aqueous phase to give the desired product as a bright yellow powder (0.048 g, 0.14 mmol, 22%). ¹H NMR (400 MHz, DMSO-d₆) δ=7.78-7.75 (m, 2H), 7.72-7.64 (m, 3H), 7.38-7.30 (m, 1H), 6.88-6.84 (m, 1H), 2.48-2.44 (m, 3H).

### Example 6: Synthesis of 6-methyl-5-methylsulfonyl-4-oxo-1-[4-(trifluoromethoxy) phenyl]cinnoline-3-carboxylic acid (Compound P6)

Step 1: Synthesis of ethyl 3-(3-bromo-2,6-difluoro-phenyl)-3-oxo-propanoate

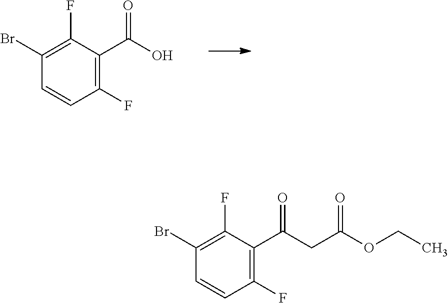 [SMILES: O=C(O)c1c(F)ccc(Br)c1F ; CCOC(=O)CC(=O)c1c(F)ccc(Br)c1F]

To a solution of 3-bromo-2,6-difluoro-benzoic acid (18 g, 76.0 mmol) in tetrahydrofuran (1.85 mmol) at 0° C. was added 1,1′-carbonyldiimidazole (83.5 mmol) portionwise. The reaction mixture was warmed to room temperature and stirred for 1 hour. The reaction mixture was then added dropwise into a suspension of magnesium chloride (114.0 mmol) and ethyl potassium malonate (114.0 mmol) in tetrahydrofuran (1860 mmol). The reaction mixture was heated at 50° C. for 5 hours. The cooled reaction mixture quenched with 2M aqueous hydrochloric acid and extracted into ethyl acetate (3×100 mL). The combined organic extracts were washed with saturated aqueous sodium hydrogen carbonate solution then brine, dried over sodium sulfate and evaporated to dryness under reduced pressure. The crude residue was purified by flash chromatography on silica gel using a gradient of 0-15% ethyl acetate in cyclohexane as eluent to give desired product as a mixture of keto-enol isomers (15 g).
Step 2: Synthesis of ethyl (2E)-3-(3-bromo-2,6-difluoro-phenyl)-3-oxo-2-[[4-(trifluoromethoxy) phenyl]hydrazono]propanoate

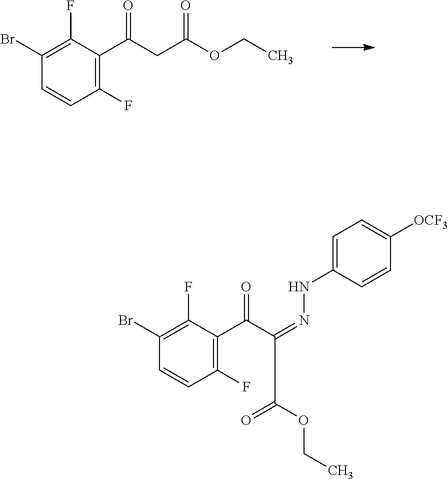 [SMILES: CCOC(=O)CC(=O)c1c(F)ccc(Br)c1F ; CCOC(=O)/C(=N/Nc1ccc(OC(F)(F)F)cc1)C(=O)c1c(F)ccc(Br)c1F]

To a cooled (0° C.) mixture of 4-(trifluoromethoxy)aniline (52.3 mmol) in 6M aqueous hydrochloric acid (261 mmol) was added dropwise over 10 minutes a solution of sodium nitrite (57.5 mmol) in water (2 mL/mmol). This was stirred at 0° C. for 60 minutes before being added dropwise over 10 minutes to a cooled (0° C.) solution of ethyl 3-(3-bromo-2,6-difluoro-phenyl)-3-oxo-propanoate (15.0 g, 48.8 mmol) and potassium acetate (244.2 mmol) in methanol (2 mL/mmol) and water (48.8 mmol, 5 mol/L). The reaction mixture was stirred at room temperature for 2 hours after which the reaction mixture was diluted with water (100 mL) and extracted into tert-butyl methyl ether (3×250 mL). The combined organic extracts were washed with brine, dried over sodium sulfate, filtered, and evaporated to dryness under reduced pressure to give desired product as a yellow solid (22 g).

Step 3: Synthesis of ethyl 6-bromo-5-fluoro-4-oxo-1-[4-(trifluoromethoxy)phenyl] cinnoline-3-carboxylate (and ethyl 8-bromo-5-fluoro-4-oxo-1-[4-(trifluoromethoxy)phenyl] cinnoline-3-carboxylate)

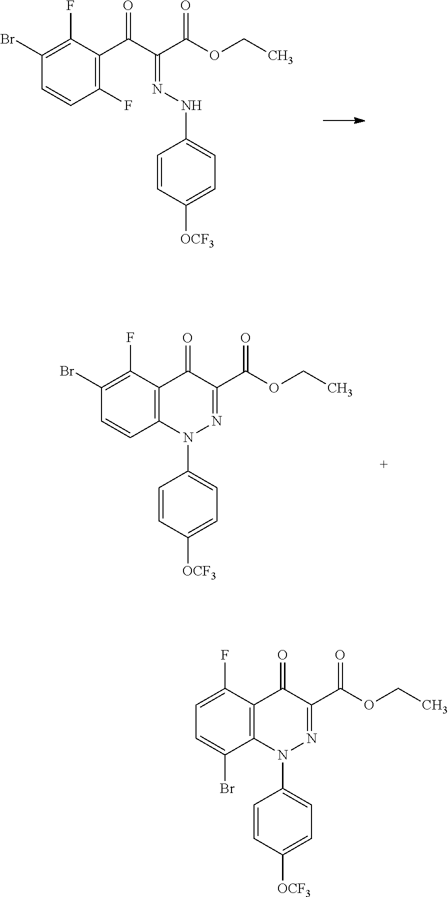 [SMILES: CCOC(=O)/C(=N\Nc1ccc(OC(F)(F)F)cc1)C(=O)c1c(F)ccc(Br)c1F ; CCOC(=O)c1nn(-c2ccc(OC(F)(F)F)cc2)c2ccc(Br)c(F)c2c1=O ; CCOC(=O)c1nn(-c2ccc(OC(F)(F)F)cc2)c2c(Br)ccc(F)c2c1=O]

To a solution of ethyl (2Z)-3-(3-bromo-2,6-difluoro-phenyl)-3-oxo-2-[[4-(trifluoromethoxy)phenyl]hydrazono]propanoate (8.0 g, 16.2 mmol) in tetrahydrofuran (160 mL) at 0° C. and under nitrogen was added portionwise a 60% suspension of sodium hydride in mineral oil (24.2 mmol). The reaction mixture was stirred at 0° C. for 4 hours. The reaction mixture was quenched by addition of ice-cold water, acidified with 1 M aqueous hydrochloric acid and extracted into ethyl acetate. The combined organic extracts were washed with brine, dried over sodium sulfate, filtered and evaporated to dryness under reduced pressure. The crude residue was purified by flash chromatography on silica gel using ethyl acetate in cyclohexane as eluent to give desired product isomer (5.1 g). ¹H NMR (400 MHz, CDCl₃): 1.41 (t, 3H), 4.45 (q, 2H), 6.88 (dd, 1H), 7.49-7.44 (m, 2H), 7.58-7.53 (m, 2H), 7.74 (dd, 1H) Step 4: Synthesis of ethyl 6-bromo-5-methylsulfanyl-4-oxo-1-[4-(trifluoromethoxy)phenyl]cinnoline-3-carboxylate (and ethyl 5,6-bis(methylsulfanyl)-4-oxo-1-[4-(trifluoromethoxy)phenyl]cinnoline-3-carboxylate)

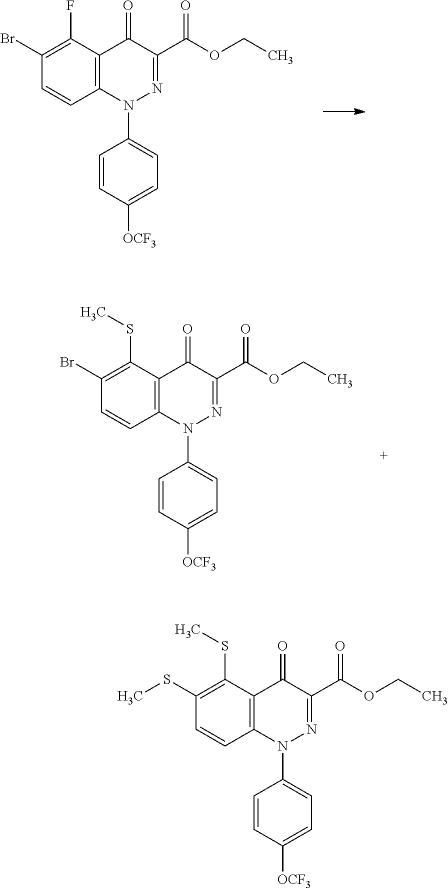

To a solution of ethyl 6-bromo-5-fluoro-4-oxo-1-[4-(trifluoromethoxy)phenyl]cinnoline-3-carboxylate (2.5 g, 5.3 mmol) in N,N-dimethylformamide (7 mL/g) at room temperature and under nitrogen was added sodium;methanethiol (1.2 equiv., 6.3 mmol). The reaction mixture was stirred at room temperature for 3 hours. The reaction mixture was quenched by addition of water (200 mL), acidified with 1M aqueous hydrochloric acid and extracted into ethyl acetate (3×300 mL). The combined organic extracts were washed with brine, dried over sodium sulfate, filtered, and evaporated to dryness under reduced pressure. The crude residue was purified by flash chromatography on silica gel using a gradient of 0 to 20% ethyl acetate in cyclohexane as eluent to give desired product as a yellow solid (2.0 g).

Step 5: Synthesis of ethyl 6-bromo-5-methylsulfonyl-4-oxo-1-[4-(trifluoromethoxy)phenyl]cinnoline-3-carboxylate

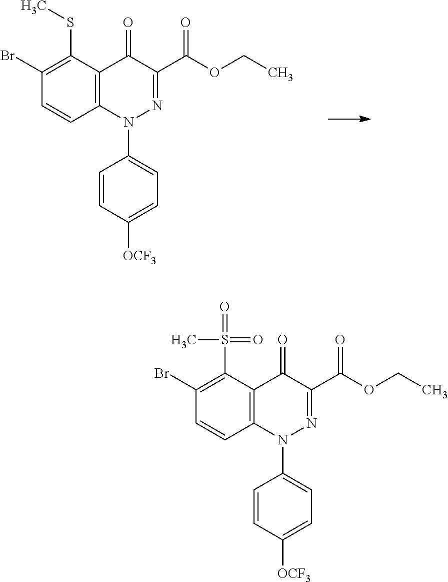 [SMILES: CCOC(=O)c1nn(-c2ccc(OC(F)(F)F)cc2)c2ccc(Br)c(SC)c2c1=O ; CCOC(=O)c1nn(-c2ccc(OC(F)(F)F)cc2)c2ccc(Br)c(S(C)(=O)=O)c2c1=O]

To a solution of ethyl 6-bromo-5-methylsulfanyl-4-oxo-1-[4-(trifluoromethoxy)phenyl]cinnoline-3-carboxylate (5.4 g, 11 mmol) in trifluoromethylbenzene (10 mL/mmol) at room temperature and under nitrogen was added 3-chloroperoxybenzoic acid (24 mmol, 70 mass). The reaction mixture was stirred at room temperature for 4 hours. The reaction mixture was diluted with water (200 mL) and extracted into ethyl acetate (3×200 mL). The combined organic extracts were washed with saturated bicarbonate solution (3×100 mL) and brine (200 mL) then dried over sodium sulphate, filtered, and evaporated to dryness under reduced pressure. The crude residue was purified by flash chromatography on silica gel using ethyl acetate in cyclohexane as eluent to give desired product (4.6 g). ¹H NMR (400 MHz, CDCl₃): 1.40 (t, 3H), 3.76 (s, 3H), 4.46 (q, 2H), 7.16 (d, 1H), 7.38-7.63 (m, 4H), 7.82 (d, 1H)
Step 6: Synthesis of ethyl 6-methyl-5-methylsulfonyl-4-oxo-1-[4-(trifluoromethoxy)phenyl]cinnoline-3-carboxylate

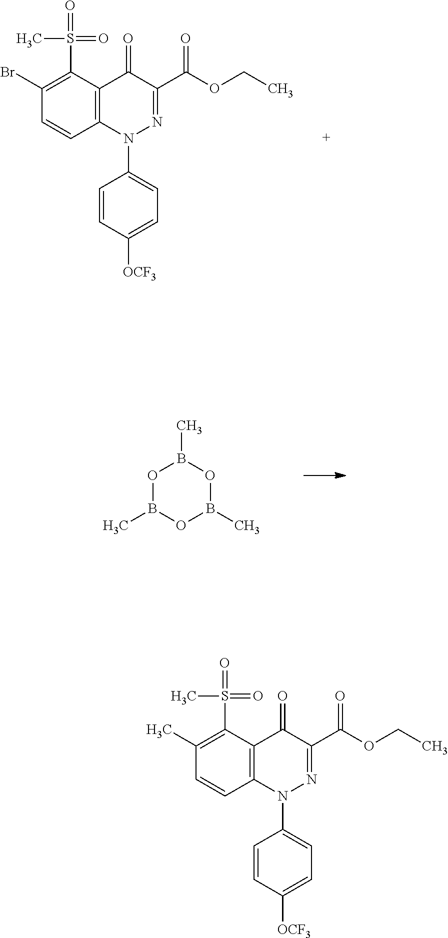

To a solution of ethyl 6-bromo-5-methylsulfonyl-4-oxo-1-[4-(trifluoromethoxy)phenyl]cinnoline-3-carboxylate (500 mg, 0.934 mmol) in diethylene dioxide (30 mL/g) was added sequentially 2,4,6-trimethyl-1,3,5,2,4,6-trioxatriborinane (2.34 mmol), sodium carbonate (1.87 mmol) and water (1 mL/g) and the resultant reaction mixture was degassed by bubbling through nitrogen for 10 minutes. The PdCI2(dppf).DCM (0.140 mmol) was added and the reaction mixture was heated at 85° C. for 20 hours. The reaction mixture was poured onto ice and diluted with water (100 mL) then acidified with 1M aqueous hydrochloric acid and extracted into ethyl acetate (3×50 mL). The combined organic extracts were washed with brine (100 mL), dried over sodium sulfate, filtered, and evaporated to dryness under reduced pressure. The crude residue was purified by flash chromatography on silica gel using a gradient of 0 to 20% ethyl acetate in cyclohexane as eluent to give desired product (0.230 g). ¹H NMR (400 MHz, CDCl₃): 1.40 (t, 3H), 2.82 (s, 3H), 3.77 (s, 3H), 4.46 (q, 2H), 7.24 (d, 1H), 7.44-7.49 (m, 3H), 7.50-7.56 (m, 2H)
Step 7: Synthesis of 6-methyl-5-methylsulfonyl-4-oxo-1-[4-(trifluoromethoxy)phenyl]cinnoline-3-carboxylic acid

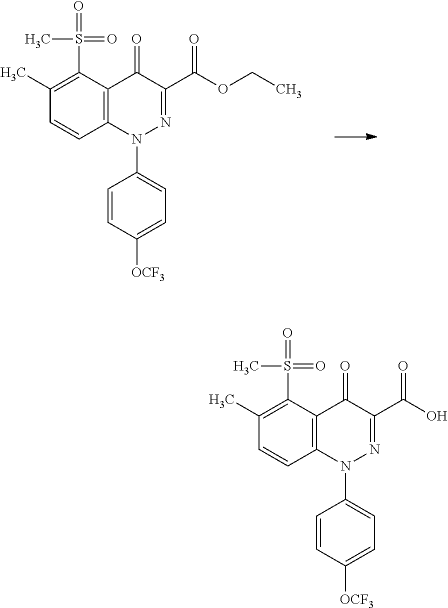

To a solution of ethyl 6-methyl-5-methylsulfonyl-4-oxo-1-[4-(trifluoromethoxy)phenyl]cinnoline-3-carboxylate (180 mg, 0.383 mmol) in tetrahydrofuran (15 mL/g) was added a solution of lithium hydroxide hydrate (1.53 mmol) in water (2 mL/g). The reaction mixture was stirred at room temperature for 2 hours. The reaction mixture was diluted with water (100 mL) and washed with ethyl acetate. The aqueous phase was acidified by addition of 1 M aqueous hydrochloric acid and then extracted into ethyl acetate. The combined organic extracts were washed with brine, dried over sodium sulfate, filtered, and evaporated to dryness under reduced pressure to give desired product as a white solid (0.150 g). ¹H NMR (400 MHz, DMSO-d6): 2.73 (s, 3H), 3.75 (s, 3H), 7.34 (d, 1H), 7.69 (d, 3H), 7.85 (d, 2H), 13.48-13.71 (brs, 1H)

### Example 7: Synthesis of 7-methyl-5-methylsulfonyl-4-oxo-1-[4-(trifluoromethoxy)phenyl]cinnoline-3-carboxylic acid (Compound P7)

Step 1: Synthesis of 4-bromo-2-fluoro-6-methylsulfanyl-benzoic acid

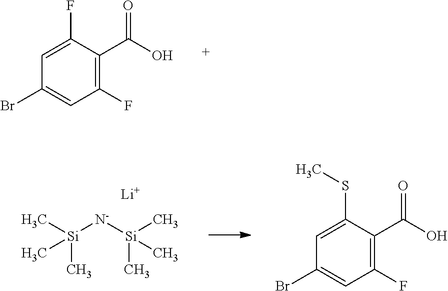

To a solution of 4-bromo-2,6-difluoro-benzoic acid (1.0 g, 4.22 mmol) in tetrahydrofuran (10 mL/g) at 0° C. was added lithium bis(trimethylsilyl)azanide (4.64 mmol,). The reaction mixture was stirred at 0° C. for 20 minutes before addition of sodium methanethiol (4.64 mmol). The resultant mixture was heated at 80° C. for 3 hours. The cooled reaction mixture was acidified by addition of 1M aqueous hydrochloric acid and diluted with ethyl acetate and water. The organic phase was washed with brine, dried over sodium sulfate, filtered and evaporated to dryness under reduced pressure to give desired product. ¹H NMR (400 MHz, CDCl₃): 2.48-2.51 (m, 3H), 7.08-7.18 (m, 1H), 7.19 (s, 1H) Step 2: Synthesis of ethyl 3-(4-bromo-2-fluoro-6-methylsulfanyl-phenyl)-3-oxo-propanoate

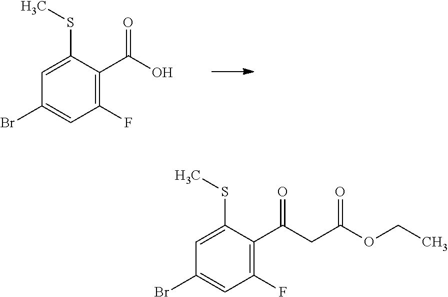 [SMILES: CSc1cc(Br)cc(F)c1C(=O)O ; CCOC(=O)CC(=O)c1c(F)cc(Br)cc1SC]

To a solution of 4-bromo-2-fluoro-6-methylsulfanyl-benzoic acid (1.1 g) in tetrahydrofuran (100 mmol) at 0° C. was added portionwise 1,1′-carbonyldiimidazole (5.0 mmol). The reaction mixture was warmed to room temperature and stirred for 1 hour. The reaction mixture was then added to a suspension of magnesium chloride (6.2 mmol) and ethyl potassium malonate (6.2 mmol) in tetrahydrofuran (100 mmol). The reaction mixture was heated at 50° C. for 18 hours. The cooled reaction mixture was quenched by addition of 2M aqueous hydrochloric acid and extracted into ethyl acetate. The combined organic extracts were washed with saturated aqueous sodium hydrogen carbonate solution then dried over sodium sulfate, filtered, and evaporated to dryness under reduced pressure. The crude residue was purified by flash chromatography on silica gel using a gradient of 15 to 20% ethyl acetate in cyclohexane as eluent to give desired product as a colourless liquid.
Step 3: Synthesis of ethyl (2E)-3-(4-bromo-2-fluoro-6-methylsulfanyl-phenyl)-3-oxo-2-[[4-(trifluoromethoxy)phenyl]hydrazono]propanoate

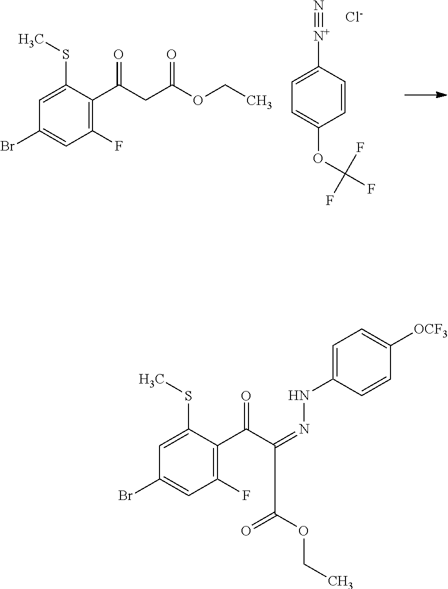 [SMILES: CCOC(=O)CC(=O)c1c(F)cc(Br)cc1SC ; N#[N+]c1ccc(OC(F)(F)F)cc1 ; [Cl-] ; CCOC(=O)/C(=N/Nc1ccc(OC(F)(F)F)cc1)C(=O)c1c(F)cc(Br)cc1SC]

To 6M aqueous hydrochloric acid (20.9 mmol) was added 4-(trifluoromethoxy)aniline (4.18 mmol). The resultant mixture was cooled to 0° C. and in an ice bath and to it was added dropwise a solution of sodium nitrite (4.60 mmol) in water (2 mL/mmol). The resultant mixture was stirred at 0° C. for 30 minutes before being added dropwise over 10 minutes to a solution of ethyl 3-(4-bromo-2-fluoro-6-methylsulfanyl-phenyl)-3-oxo-propanoate (1.0 g) and potassium acetate (14.9 mmol) in methanol (2.0 mL/mmol) and water (2.98 mmol) at 0° C. On completion of addition, the reaction mixture was stirred at room temperature for 2 hours. The gummy brownish mass formed was extracted into ethyl acetate, washed with brine, dried over sodium sulfate, filtered, and evaporated to dryness under reduced pressure to afford crude desired product.
Step 4: Synthesis of ethyl 7-bromo-5-methylsulfanyl-4-oxo-1-[4-(trifluoromethoxy)phenyl]cinnoline-3-carboxylate

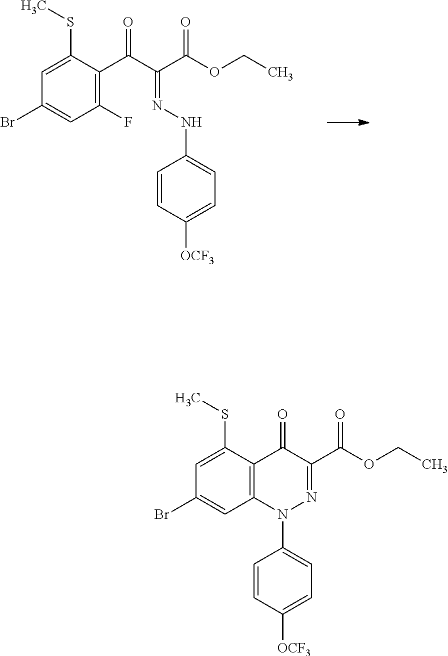 [SMILES: CCOC(=O)/C(=N\Nc1ccc(OC(F)(F)F)cc1)C(=O)c1c(F)cc(Br)cc1SC ; CCOC(=O)c1nn(-c2ccc(OC(F)(F)F)cc2)c2cc(Br)cc(SC)c2c1=O]

To a solution of ethyl (2Z)-3-(4-bromo-2-fluoro-6-methylsulfanyl-phenyl)-3-oxo-2-[[4-(trifluoromethoxy)phenyl]hydrazono]propanoate (900 mg) in N,N-dimethylformamide (10 mL) was added potassium carbonate (1.89 mmol). The reaction mixture was heated at 100° C. for 2.5 hours. To the cooled reaction mixture was added cold water and the precipitated solid was collected by filtration and air-dried to give the desired product. ¹H NMR (400 MHz, DMSO-d6): 1.22-1.30 (m, 3H), 2.45-2.47 (m, 3H), 4.30 (d, 2H), 6.82 (d, 1H), 7.30 (d, 1H), 7.67 (d, 2H), 7.83 (d, 2H)
Step 5: Synthesis of ethyl 7-bromo-5-methylsulfonyl-4-oxo-1-[4-(trifluoromethoxy)phenyl]cinnoline-3-carboxylate

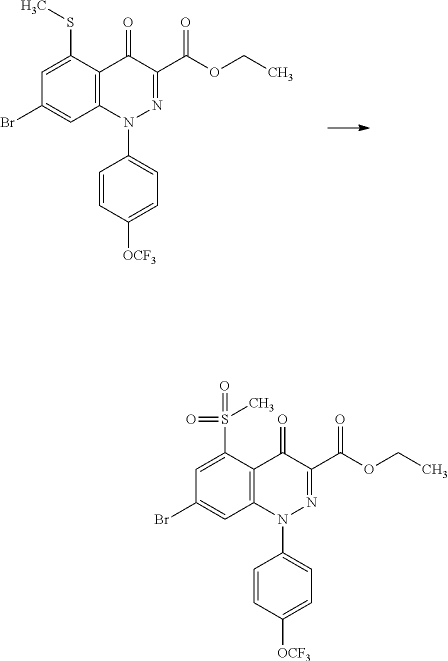 [SMILES: CCOC(=O)c1nn(-c2ccc(OC(F)(F)F)cc2)c2cc(Br)cc(SC)c2c1=O ; CCOC(=O)c1nn(-c2ccc(OC(F)(F)F)cc2)c2cc(Br)cc(S(C)(=O)=O)c2c1=O]

To a solution of ethyl 7-bromo-5-methylsulfanyl-4-oxo-1-[4-(trifluoromethoxy)phenyl]cinnoline-3-carboxylate (650 mg) in acetonitrile (20 mL) at 0° C. was added 3-chlorobenzenecarboperoxoic acid (2.84 mmol, 70 mass %). The reaction mixture was stirred at room temperature for 18 hours. The reaction mixture was quenched by addition of saturated aqueous potassium carbonate solution (20 mL) and water (20 mL) and then extracted into ethyl acetate. The combined organic extracts were washed with brine, dried over sodium sulfate, filtered, and evaporated to dryness under reduced pressure. The crude residue was purified by flash chromatography on silica gel using a gradient of 40 to 50% ethyl acetate in cyclohexane as eluent to give desired product. ¹H NMR (400 MHz, DMSO-d₆): 1.23-1.33 (m, 3H), 3.70 (s, 3H), 4.34 (q, 2H), 7.63 (d, 1H), 7.69 (d, 2H), 7.82-7.90 (m, 2H), 8.25 (d, 1H)
Step 6: Synthesis of ethyl 7-methyl-5-methylsulfonyl-4-oxo-1-[4-(trifluoromethoxy)phenyl]cinnoline-3-carboxylate

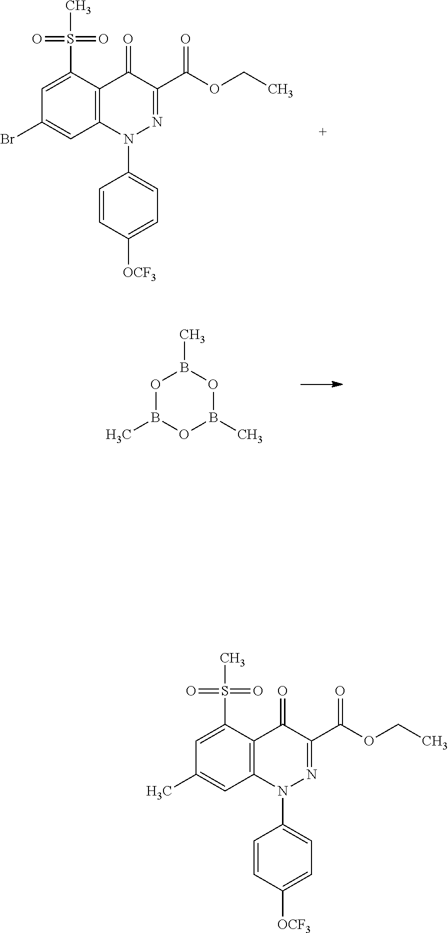

To a solution of ethyl 7-bromo-5-methylsulfonyl-4-oxo-1-[4-(trifluoromethoxy)phenyl]cinnoline-3-carboxylate (500 mg) in diethylene dioxide (30 mL/g) was added sequentially 2,4,6-trimethyl-1,3,5,2,4,6-trioxatriborinane (2.34 mmol), sodium carbonate (1.87 mmol) and water (1 mL/g). The reaction mixture was degassed by bubbling through with nitrogen for 15 minutes. PdCI2(dppf).DCM (0.14 mmol) was added and the reaction mixture was heated at 100° C. for 2 hours. The reaction mixture was diluted with ethyl acetate and washed with water then brine, then dried over sodium sulfate, filtered, and evaporated to dryness under reduced pressure. The crude residue was purified by flash chromatography on silica gel using a gradient of 40 to 50% ethyl acetate in cyclohexane as eluent to give desired product. ¹H NMR (400 MHz, CDCl₃): 7.56-7.50 (m, 2H), 7.49-7.44 (m, 3H), 7.24 (d, 1H), 4.46 (q, 2H), 3.77 (s, 3H), 2.82 (s, 3H), 1.40 (t, 3H)
Step 7: Synthesis of 7-methyl-5-methylsulfonyl-4-oxo-1-[4-(trifluoromethoxy)phenyl]cinnoline-3-carboxylic acid

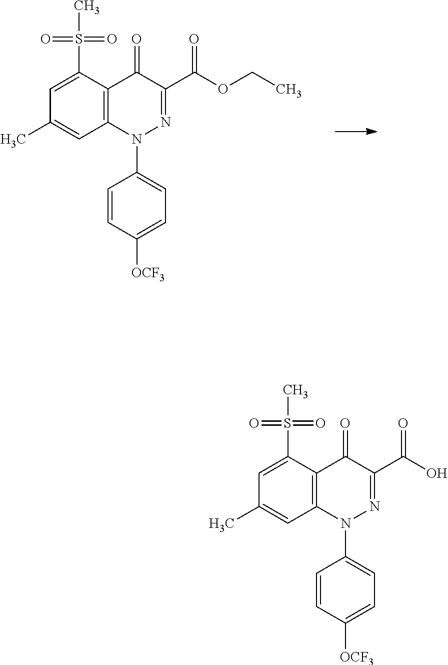

To a solution of ethyl 7-methyl-5-methylsulfonyl-4-oxo-1-[4-(trifluoromethoxy)phenyl]cinnoline-3-carboxylate (200 mg) in tetrahydrofuran (10 mL) was added a suspension of lithium hydroxide hydrate (3 equiv., 1.276 mmol) in water (1 mL/g). The reaction mixture was stirred at room temperature for 18 hours. The reaction mixture was acidified by the addition of 2M aqueous hydrochloric acid and diluted with additional water. The precipitated solid was collected by filtration, washed with tert-butyl methyl ether and air-dried to give the desired product. ¹H NMR (400 MHz, DMSO-d6): 14.26-13.44 (m, 1H), 8.16-8.14 (m, 1H), 7.70 (d, 2H), 7.84 (d, 2H), 7.35 (s, 1H), 3.67 (s, 3H), 2.49-2.47 (m, 3H)

TABLE 2
¹H NMR and LC/MS data for selected compounds of Table 1

| Cpd | Compound | | |
|---|---|---|---|
| No. | Name | Structure & ¹H NMR Data | LC/MS |
| P1 | 5-methylsulfanyl-4-oxo-1-[4- (trifluoromethoxy)phenyl]cin- noline-3-carboxylic acid | 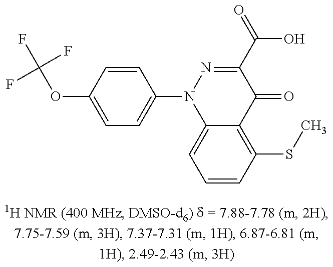 [SMILES: CSc1cccc2c1c(=O)c(C(=O)O)nn2-c1ccc(OC(F)(F)F)cc1] | Rₜ = 1.03 min; MS: m/z = 397 (M + H) |
| P2 | 5-methylsulfonyl-4-oxo-1-[4- (trifluoromethoxy)phenyl]cin- noline-3-carboxylic acid | 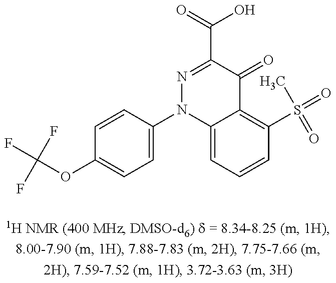 [SMILES: CS(=O)(=O)c1cccc2c1c(=O)c(C(=O)O)nn2-c1ccc(OC(F)(F)F)cc1] | Rₜ = 0.76 min; MS: m/z = 429 (M + H) |
| P3 | methyl 5-methylsulfonyl-4- oxo-1-[4- (trifluoromethoxy)phenyl]cin- noline-3-carboxylate | 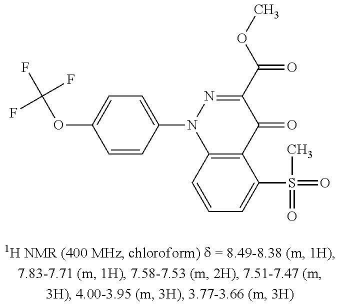 [SMILES: COC(=O)c1nn(-c2ccc(OC(F)(F)F)cc2)c2cccc(S(C)(=O)=O)c2c1=O] | Rₜ = 0.90 min; MS: m/z = 443 (M + H) |
| P4 | 1-(4-chlorophenyl)-5- methylsulfanyl-4-oxo- cinnoline-3-carboxylic acid | 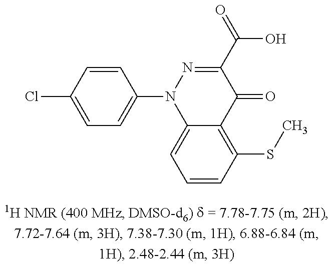 [SMILES: CSc1cccc2c1c(=O)c(C(=O)O)nn2-c1ccc(Cl)cc1] | Rₜ = 0.94 min; MS: m/z = 347 (M + H) |
| P5 | 1-(4-chlorophenyl)-5- methylsulfonyl-4-oxo- cinnoline-3-carboxylic acid | 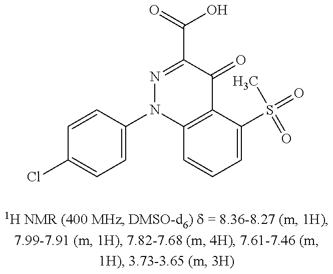 [SMILES: CS(=O)(=O)c1cccc2c1c(=O)c(C(=O)O)nn2-c1ccc(Cl)cc1] | Rₜ = 0.64 min; MS: m/z = 379 (M + H) |
| P6 | 6-methyl-5-methylsulfonyl-4- oxo-1-[4-(trifluoromethoxy) phenyl]cinnoline-3- carboxylic acid | 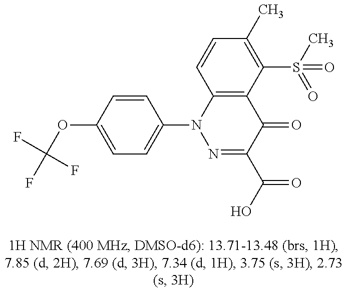 [SMILES: Cc1ccc2c(c1S(C)(=O)=O)c(=O)c(C(=O)O)nn2-c1ccc(OC(F)(F)F)cc1] | |
| P7 | 7-methyl-5-methylsulfonyl-4- oxo-1-[4-(trifluoromethoxy) phenyl]cinnoline-3- carboxylic acid | 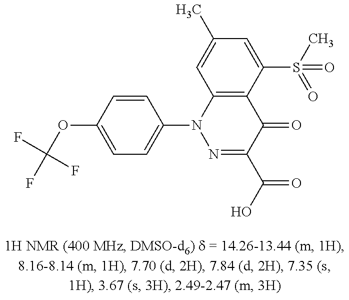 [SMILES: Cc1cc(S(C)(=O)=O)c2c(=O)c(C(=O)O)nn(-c3ccc(OC(F)(F)F)cc3)c2c1] | |
| P8 | ethyl 6-methyl-5- methylsulfonyl-4-oxo-1-[4- (trifluoromethoxy)phenyl]cin- noline-3-carboxylic acid | 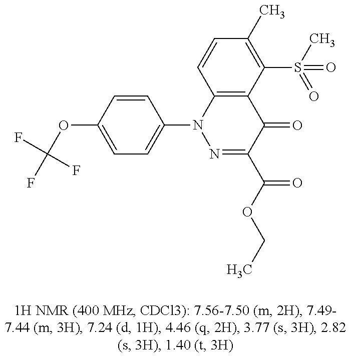 [SMILES: CCOC(=O)c1nn(-c2ccc(OC(F)(F)F)cc2)c2ccc(C)c(S(C)(=O)=O)c2c1=O] | |
| P9 | ethyl 7-methyl-5- methylsulfonyl-4-oxo-1-[4- (trifluoromethoxy)phenyl]cin- noline-3-carboxylate | 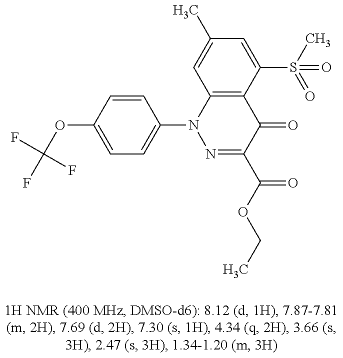 [SMILES: CCOC(=O)c1nn(-c2ccc(OC(F)(F)F)cc2)c2cc(C)cc(S(C)(=O)=O)c2c1=O] | |
| P10 | ethyl 6-bromo-5- methysulfonyl-4-oxo-1-[4- (trifluoromethoxy)phenyl]cin- noline-3-carboxylate | 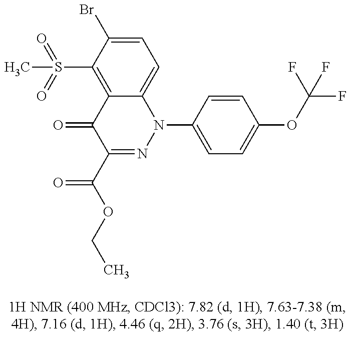 [SMILES: CCOC(=O)c1nn(-c2ccc(OC(F)(F)F)cc2)c2ccc(Br)c(S(C)(=O)=O)c2c1=O] | |
| P11 | ethyl 7-bromo-5- methylsulfonyl-4-oxo-1-[4- (trifluoromethoxy)phenyl]cin- noline-3-carboxylate | 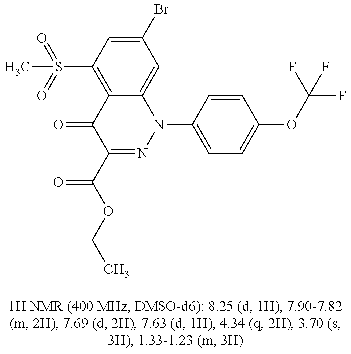 [SMILES: CCOC(=O)c1nn(-c2ccc(OC(F)(F)F)cc2)c2cc(Br)cc(S(C)(=O)=O)c2c1=O] | |
| P12 | 6-bromo-5-methylsulfonyl-4- oxo-1-[4- (trifluoromethoxy)phenyl]cin- noline-3-carboxylic acid | 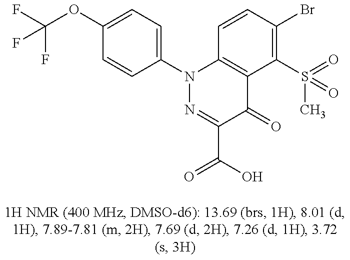 [SMILES: CS(=O)(=O)c1c(Br)ccc2c1c(=O)c(C(=O)O)nn2-c1ccc(OC(F)(F)F)cc1] | |
| P13 | 7-bromo-5-methylsulfonyl-4- oxo-1-[4- (trifluoromethoxy)phenyl]cin- noline-3-carboxylic acid | 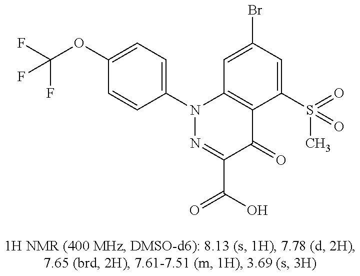 [SMILES: CS(=O)(=O)c1cc(Br)cc2c1c(=O)c(C(=O)O)nn2-c1ccc(OC(F)(F)F)cc1] | |
| P14 | 7-isobutyl-5-methylsulfonyl- 4-oxo-1-[4- (trifluoromethoxy)phenyl]cin- noline-3-carboxylic acid | 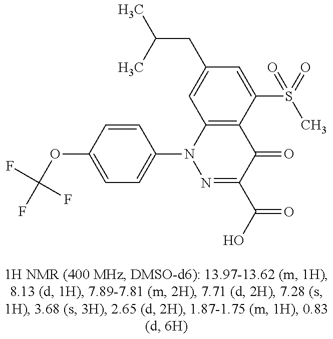 [SMILES: CC(C)Cc1cc(S(C)(=O)=O)c2c(=O)c(C(=O)O)nn(-c3ccc(OC(F)(F)F)cc3)c2c1] | |
| P15 | 6-isobutyl-5-methylsulfonyl- 4-oxo-1-[4- (trifluoromethoxy)phenyl]cin- noline-3-carboxylic acid | 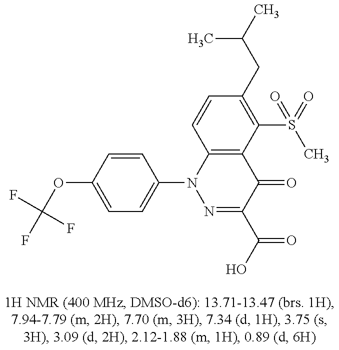 [SMILES: CC(C)Cc1ccc2c(c1S(C)(=O)=O)c(=O)c(C(=O)O)nn2-c1ccc(OC(F)(F)F)cc1] | |
| P16 | 6-cyano-5-methysulfonyl-4- oxo-1-[4- (trifluoromethoxy)phenyl]cin- noline-3-carboxylic acid | 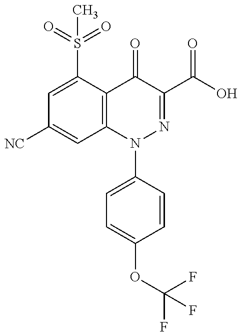 [SMILES: CS(=O)(=O)c1cc(C#N)cc2c1c(=O)c(C(=O)O)nn2-c1ccc(OC(F)(F)F)cc1] | |
| P17 | 7-cyano-5-methylsulfonyl-4- oxo-1-[4- (trifluoromethoxy)phenyl]cin- noline-3-carboxylic acid | 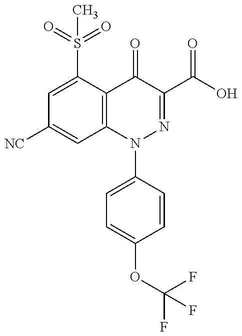 [SMILES: CS(=O)(=O)c1cc(C#N)cc2c1c(=O)c(C(=O)O)nn2-c1ccc(OC(F)(F)F)cc1] | |
| P18 | 6-methoxy-5-methylsulfonyl- 4-oxo-1-[4- (trifluoromethoxy)phenyl]cin- noline-3-carboxylic acid | 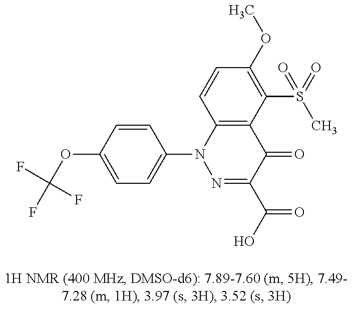 [SMILES: COc1ccc2c(c1S(C)(=O)=O)c(=O)c(C(=O)O)nn2-c1ccc(OC(F)(F)F)cc1] | |
| P19 | 7-methoxy-5-methylsulfonyl- 4-oxo-1-[4- (trifluoromethoxy)phenyl]cin- noline-3-carboxylic acid | 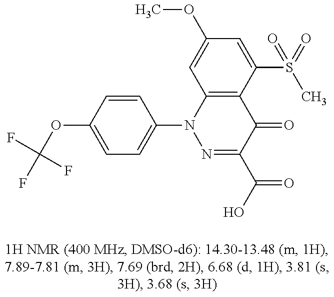 [SMILES: COc1cc(S(C)(=O)=O)c2c(=O)c(C(=O)O)nn(-c3ccc(OC(F)(F)F)cc3)c2c1] | |
| P20 | 6-fluoro-5-methylsulfonyl-4- oxo-1-[4- (trifluoromethoxy)phenyl]cin- noline-3-carboxylic acid | 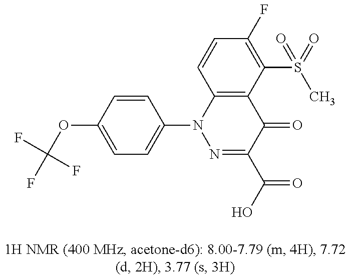 [SMILES: CS(=O)(=O)c1c(F)ccc2c1c(=O)c(C(=O)O)nn2-c1ccc(OC(F)(F)F)cc1] | |
| P21 | 7-fluoro-5-methylsulfonyl-4- oxo-1-[4- (trifluoromethoxy)phenyl]cin- noline-3-carboxylic acid | 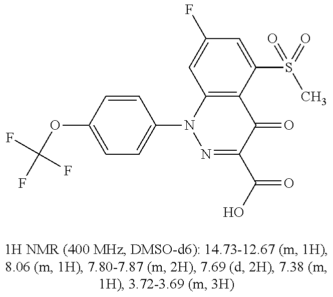 [SMILES: CS(=O)(=O)c1cc(F)cc2c1c(=O)c(C(=O)O)nn2-c1ccc(OC(F)(F)F)cc1] | |
| P22 | ethyl 5-ethylsulfonyl-4-oxo- 1-[4- (trifluoromethoxy)phenyl]cin- noline-3-carboxylate | 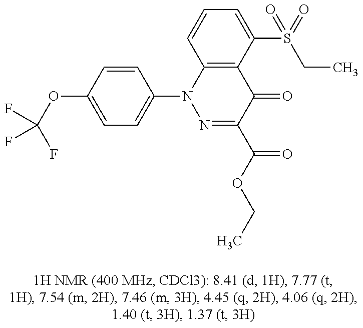 [SMILES: CCOC(=O)c1nn(-c2ccc(OC(F)(F)F)cc2)c2cccc(S(=O)(=O)CC)c2c1=O] | |
| P23 | ethyl 5-ethylsulfanyl-4-oxo- 1-[4- (trifluoromethoxy)phenyl]cin- noline-3-carboxylate | 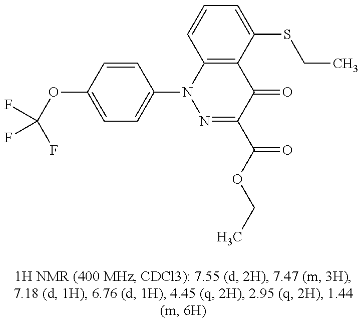 [SMILES: CCOC(=O)c1nn(-c2ccc(OC(F)(F)F)cc2)c2cccc(SCC)c2c1=O] | |
| P24 | ethyl 5-ethylsulfinyl-4-oxo-1- [4- (trifluoromethoxy)phenyl]cin- noline-3-carboxylate | 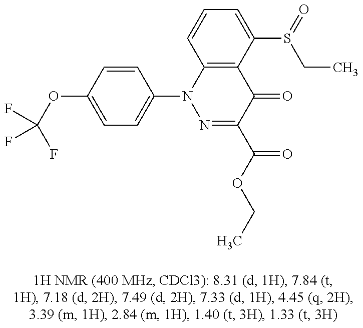 [SMILES: CCOC(=O)c1nn(-c2ccc(OC(F)(F)F)cc2)c2cccc(S(=O)CC)c2c1=O] | |
| P25 | 5-ethylsulfinyl-4-oxo-1-[4- (trifluoromethoxy)phenyl]cin- noline-3-carboxylic acid | 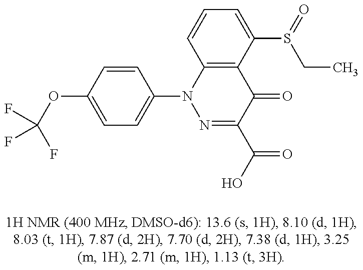 [SMILES: CCS(=O)c1cccc2c1c(=O)c(C(=O)O)nn2-c1ccc(OC(F)(F)F)cc1] | |
| P26 | 5-ethylsulfanyl-4-oxo-1-[4- (trifluoromethoxy)phenyl]cin- noline-3-carboxylic acid | 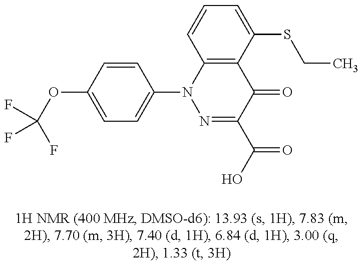 [SMILES: CCSc1cccc2c1c(=O)c(C(=O)O)nn2-c1ccc(OC(F)(F)F)cc1] | |
| P27 | 5-ethylsulfonyl-4-oxo-1-[4- (trifluoromethoxy)phenyl]cin- noline-3-carboxylic acid | 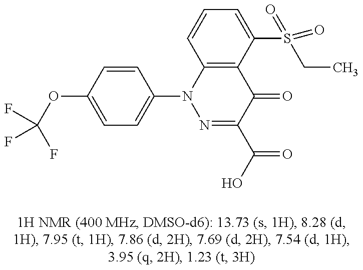 [SMILES: CCS(=O)(=O)c1cccc2c1c(=O)c(C(=O)O)nn2-c1ccc(OC(F)(F)F)cc1] | |
| P28 | ethyl 5-methylsulfinyl-4-oxo- 1-[4- (trifluoromethoxy)phenyl]cin- noline-3-carboxylate | 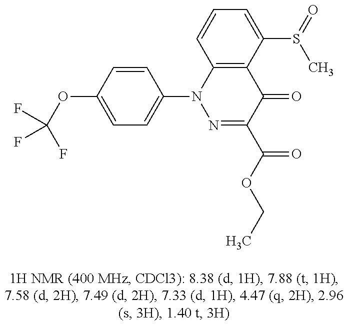 [SMILES: CCOC(=O)c1nn(-c2ccc(OC(F)(F)F)cc2)c2cccc(S(C)=O)c2c1=O] | |
| P29 | 5-cyclopropylsulfonyl-4-oxo- 1-[4- (trifluoromethoxy)phenyl]cin- noline-3-carboxylic acid | 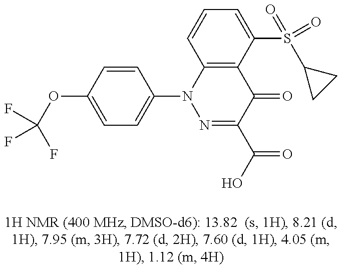 [SMILES: O=C(O)c1nn(-c2ccc(OC(F)(F)F)cc2)c2cccc(S(=O)(=O)C3CC3)c2c1=O] | |
| P30 | 5-cyclopropylsulfanyl-4-oxo- 1-[4- (trifluoromethoxy)phenyl]cin- noline-3-carboxylic acid | 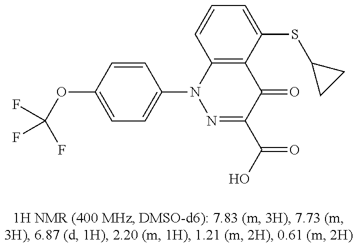 [SMILES: O=C(O)c1nn(-c2ccc(OC(F)(F)F)cc2)c2cccc(SC3CC3)c2c1=O] | |
| P31 | 5-cyclopropylsulfinyl-4-oxo- 1-[4- (trifluoromethoxy)phenyl]cin- noline-3-carboxylic acid | 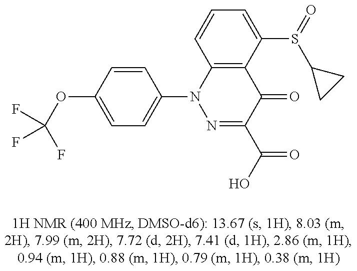 [SMILES: O=C(O)c1nn(-c2ccc(OC(F)(F)F)cc2)c2cccc(S(=O)C3CC3)c2c1=O] | |
| P32 | ethyl 5-cyclopropylsulfinyl-4- oxo-1-[4- (trifluoromethoxy)phenyl]cin- noline-3-carboxylate | 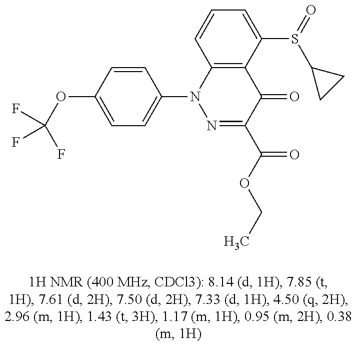 [SMILES: CCOC(=O)c1nn(-c2ccc(OC(F)(F)F)cc2)c2cccc(S(=O)C3CC3)c2c1=O] | |
| P33 | ethyl 5-cyclopropylsulfanyl- 4-oxo-1-[4- (trifluoromethoxy)phenyl]cin- noline-3-carboxylate | 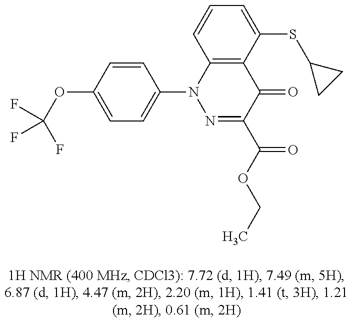 [SMILES: CCOC(=O)c1nn(-c2ccc(OC(F)(F)F)cc2)c2cccc(SC3CC3)c2c1=O] | |
| P34 | ethyl 5-cyclopropylsulfonyl- 4-oxo-1-[4- (trifluoromethoxy)phenyl]cin- noline-3-carboxylate | 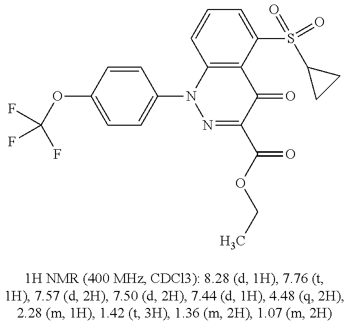 [SMILES: CCOC(=O)c1nn(-c2ccc(OC(F)(F)F)cc2)c2cccc(S(=O)(=O)C3CC3)c2c1=O] | |
| P35 | 4-oxo-5-(2,2,2- trifluoroethylsulfonyl)-1-[4- (trifluoromethoxy)phenyl]cin- noline-3-carboxylic acid | 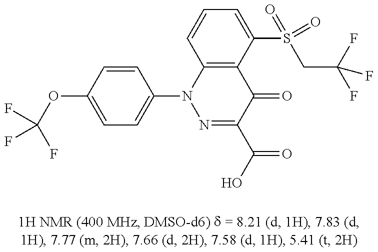 [SMILES: O=C(O)c1nn(-c2ccc(OC(F)(F)F)cc2)c2cccc(S(=O)(=O)CC(F)(F)F)c2c1=O] | |
| P36 | 4-oxo-5-(2,2,2- trifluoroethylsulfanyl)-1-[4- (trifluoromethoxy)phenyl]cin- noline-3-carboxylic acid | 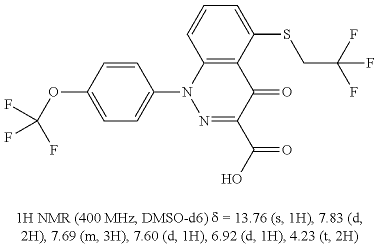 [SMILES: O=C(O)c1nn(-c2ccc(OC(F)(F)F)cc2)c2cccc(SCC(F)(F)F)c2c1=O] | |
| P37 | 4-oxo-5-(2,2,2- trifluoroethylsulfinyl)-1-[4- (trifluoromethoxy)phenyl]cin- noline-3-carboxylic acid | 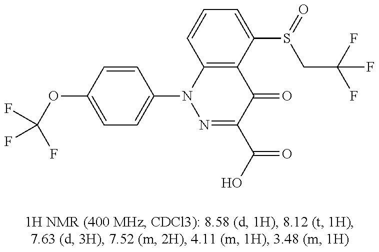 [SMILES: O=C(O)c1nn(-c2ccc(OC(F)(F)F)cc2)c2cccc(S(=O)CC(F)(F)F)c2c1=O] | |
| P38 | ethyl 4-oxo-5-(2,2,2- trifluoroethylsulfinyl)-1-[4- (trifluoromethoxy)phenyl]cin- noline-3-carboxylate | 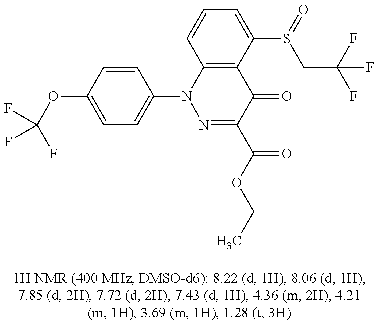 [SMILES: CCOC(=O)c1nn(-c2ccc(OC(F)(F)F)cc2)c2cccc(S(=O)CC(F)(F)F)c2c1=O] | |
| P39 | ethyl 4-oxo-5-(2,2,2- trifluoroethylsulfanyl)-1-[4- (trifluoromethoxy)phenyl]cin- noline-3-carboxylate | 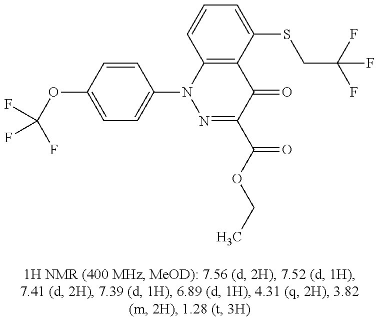 [SMILES: CCOC(=O)c1nn(-c2ccc(OC(F)(F)F)cc2)c2cccc(SCC(F)(F)F)c2c1=O] | |
| P40 | ethyl 4-oxo-5-(2,2,2- trifluoroethylsulfonyl)-1-[4- (trifluoromethoxy)phenyl]cin- noline-3-carboxylate | 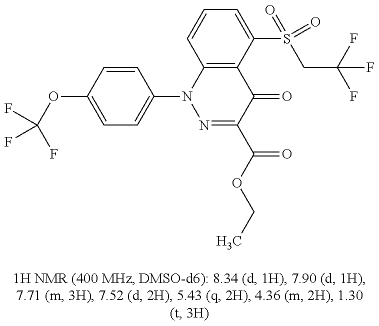 [SMILES: CCOC(=O)c1nn(-c2ccc(OC(F)(F)F)cc2)c2cccc(S(=O)(=O)CC(F)(F)F)c2c1=O] | |
| P41 | 1-(4-chloro-2-fluoro-phenyl)- 5-methylsulfonyl-4-oxo- cinnoline-3-carboxylic acid | 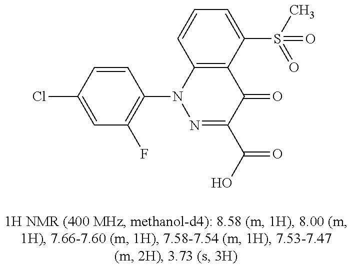 [SMILES: CS(=O)(=O)c1cccc2c1c(=O)c(C(=O)O)nn2-c1ccc(Cl)cc1F] | |
| P42 | ethyl 1-(4-chloro-2-fluoro- phenyl)-5-methylsulfonyl-4- oxo-cinnoline-3-carboxylate | 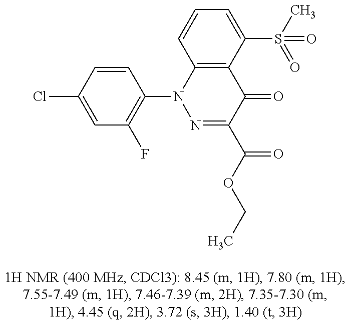 [SMILES: CCOC(=O)c1nn(-c2ccc(Cl)cc2F)c2cccc(S(C)(=O)=O)c2c1=O] | |
| P43 | 1-(2,4-dichlorophenyl)-5- methylsulfonyl-4-oxo- cinnoline-3-carboxylic acid | 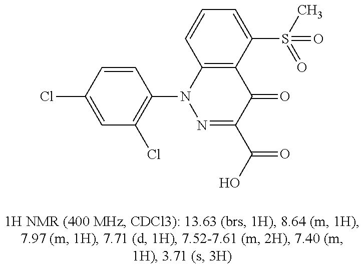 [SMILES: CS(=O)(=O)c1cccc2c1c(=O)c(C(=O)O)nn2-c1ccc(Cl)cc1Cl] | |
| P44 | ethyl 1-(2,4-dichlorophenyl)- 5-methylsulfonyl-4-oxo- cinnoline-3-carboxylate | 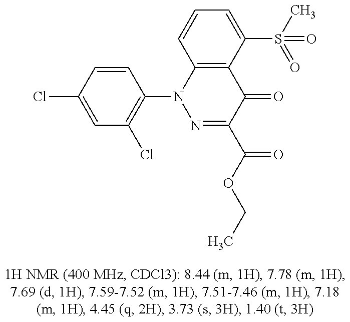 [SMILES: CCOC(=O)c1nn(-c2ccc(Cl)cc2Cl)c2cccc(S(C)(=O)=O)c2c1=O] | |
| P45 | ethyl 6-isobutyl-5- methylsulfonyl-4-oxo-1-[4- (trifluoromethoxy)phenyl]cin- noline-3-carboxylate | 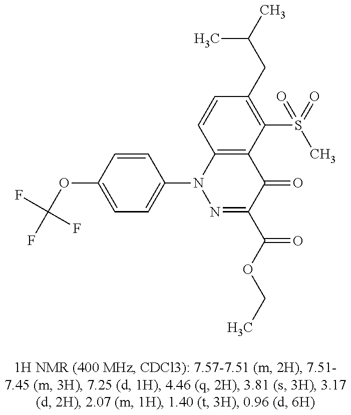 [SMILES: CCOC(=O)c1nn(-c2ccc(OC(F)(F)F)cc2)c2ccc(CC(C)C)c(S(C)(=O)=O)c2c1=O] | |
| P46 | ethyl 7-isobutyl-5- methysulfonyl-4-oxo-1-[4- (trifluoromethoxy)phenyl]cin- noline-3-carboxylate | 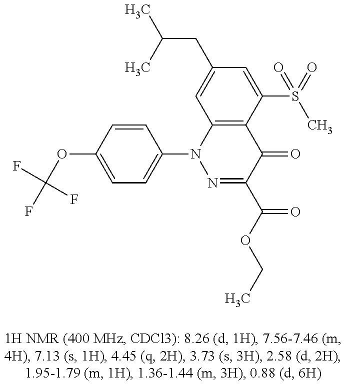 [SMILES: CCOC(=O)c1nn(-c2ccc(OC(F)(F)F)cc2)c2cc(CC(C)C)cc(S(C)(=O)=O)c2c1=O] | |
| P47 | ethyl 6-cyano-5- methylsulfonyl-4-oxo-1-[4- (trifluoromethoxy)phenyl]cin- noline-3-carboxylate | 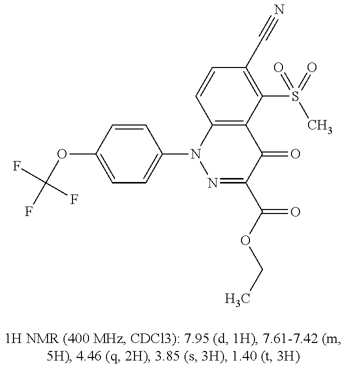 [SMILES: CCOC(=O)c1nn(-c2ccc(OC(F)(F)F)cc2)c2ccc(C#N)c(S(C)(=O)=O)c2c1=O] | |
| P48 | ethyl 7-cyano-5- methylsulfonyl-4-oxo-1-[4- (trifluoromethoxy)phenyl]cin- noline-3-carboxylate | 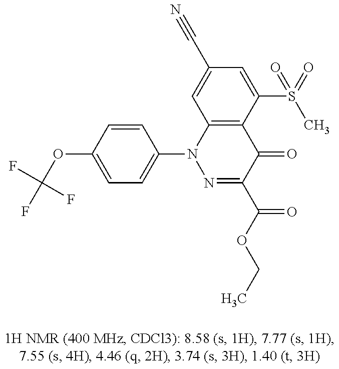 [SMILES: CCOC(=O)c1nn(-c2ccc(OC(F)(F)F)cc2)c2cc(C#N)cc(S(C)(=O)=O)c2c1=O] | |
| P49 | ethyl 6-methoxy-5- methylsulfonyl-4-oxo-1-[4- (trifluoromethoxy)phenyl]cin- noline-3-carboxylate | 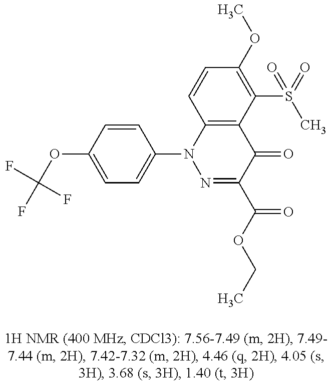 [SMILES: CCOC(=O)c1nn(-c2ccc(OC(F)(F)F)cc2)c2ccc(OC)c(S(C)(=O)=O)c2c1=O] | |
| P50 | ethyl 7-methoxy-5- methysulfonyl-4-oxo-1-[4- (trifluoromethoxy)phenyl]cin- noline-3-carboxylate | 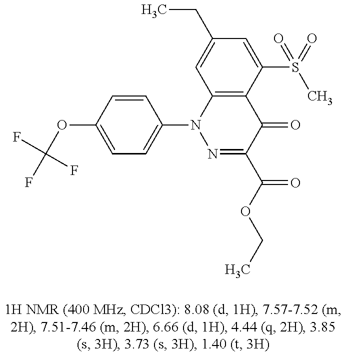 [SMILES: CCOC(=O)c1nn(-c2ccc(OC(F)(F)F)cc2)c2cc(CC)cc(S(C)(=O)=O)c2c1=O] | |
| P51 | ethyl 6-fluoro-5- methylsulfonyl-4-oxo-1-[4- (trifluoromethoxy)phenyl]cin- noline-3-carboxylate | 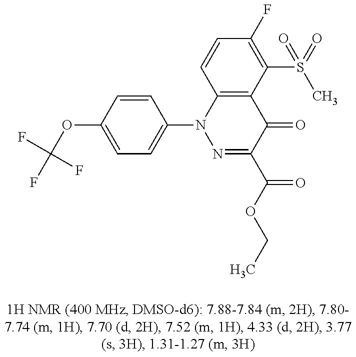 [SMILES: CCOC(=O)c1nn(-c2ccc(OC(F)(F)F)cc2)c2ccc(F)c(S(C)(=O)=O)c2c1=O] | |
| P52 | ethyl 7-fluoro-5- methylsulfonyl-4-oxo-1-[4- (trifluoromethoxy)phenyl]cin- noline-3-carboxylate | 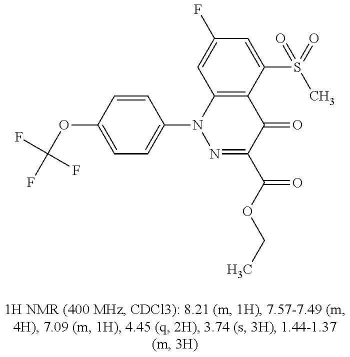 [SMILES: CCOC(=O)c1nn(-c2ccc(OC(F)(F)F)cc2)c2cc(F)cc(S(C)(=O)=O)c2c1=O] | |
| P53 | ethyl 6-bromo-5- methylsulfanyl-4-oxo-1-[4- (trifluoromethoxy)phenyl]cin- noline-3-carboxylate | 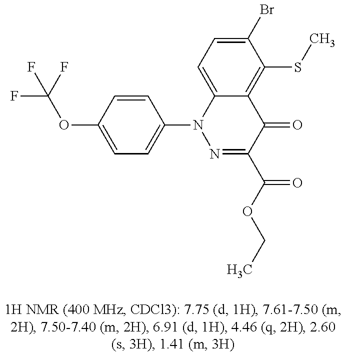 [SMILES: CCOC(=O)c1nn(-c2ccc(OC(F)(F)F)cc2)c2ccc(Br)c(SC)c2c1=O] | |
| P54 | ethyl 7-fluoro-5- methylsulfanyl-4-oxo-1-[4- (trifluoromethoxy)phenyl]cin- noline-3-carboxylate | 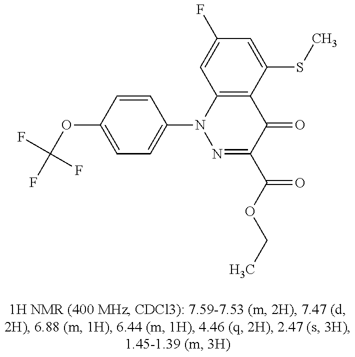 [SMILES: CCOC(=O)c1nn(-c2ccc(OC(F)(F)F)cc2)c2cc(F)cc(SC)c2c1=O] | |
| P55 | ethyl 7-cyano-5- methylsulfanyl-4-oxo-1-[4- (trifluoromethoxy)phenyl]cin- noline-3-carboxylate | 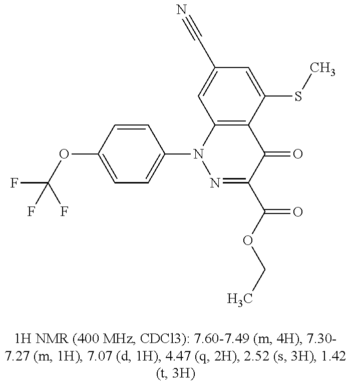 [SMILES: CCOC(=O)c1nn(-c2ccc(OC(F)(F)F)cc2)c2cc(C#N)cc(SC)c2c1=O] | |
| P56 | methyl 7-methoxy-5- methylsulfanyl-4-oxo-1-[4- (trifluoromethoxy)phenyl]cin- noline-3-carboxylate | 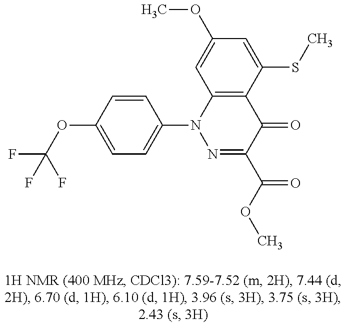 [SMILES: COC(=O)c1nn(-c2ccc(OC(F)(F)F)cc2)c2cc(OC)cc(SC)c2c1=O] | |
| P57 | ethyl 7-methoxy-5- methylsulfanyl-4-oxo-1-[4- (trifluoromethoxy)phenyl]cin- noline-3-carboxylate | 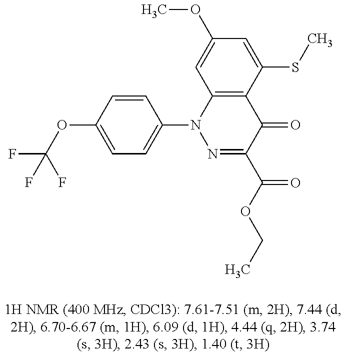 [SMILES: CCOC(=O)c1nn(-c2ccc(OC(F)(F)F)cc2)c2cc(OC)cc(SC)c2c1=O] | |
| P58 | 7-cyano-5-methylsulfanyl-4- oxo-1-[4- (trifluoromethoxy)phenyl]cin- noline-3-carboxylic acid | 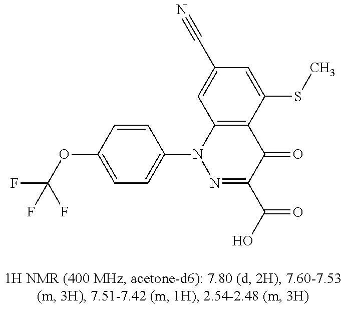 [SMILES: CSc1cc(C#N)cc2c1c(=O)c(C(=O)O)nn2-c1ccc(OC(F)(F)F)cc1] | |
| P59 | hexyl 5-methylsulfonyl-4- oxo-1-[4- (trifluoromethoxy)phenyl]cin- oline-3-carboxylate | 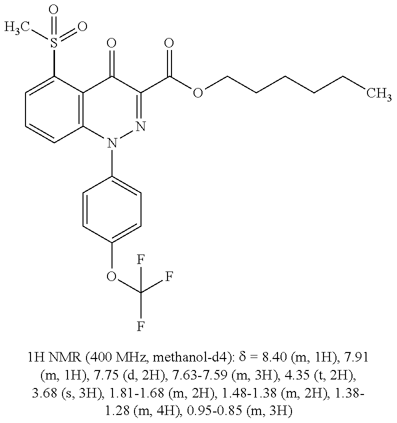 [SMILES: CCCCCCOC(=O)c1nn(-c2ccc(OC(F)(F)F)cc2)c2cccc(S(C)(=O)=O)c2c1=O] | |
| P60 | undecyl 5-methylsulfonyl-4- oxo-1-[4- (trifluoromethoxy)phenyl]cin- noline-3-carboxylate | 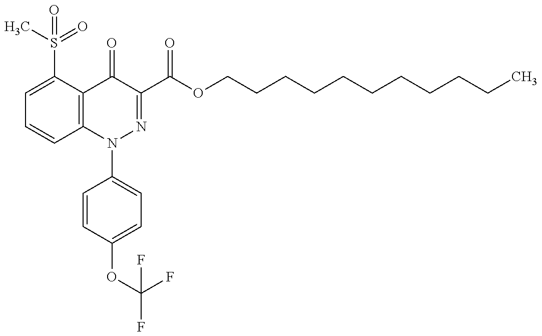 [SMILES: CCCCCCCCCCCOC(=O)c1nn(-c2ccc(OC(F)(F)F)cc2)c2cccc(S(C)(=O)=O)c2c1=O] | Rₜ = 3.31 min; MS: m/z = 583 (M + H) |
| P61 | 2-chloroethyl 5- methylsulfonyl-4-oxo-1-[4- (trifluoromethoxy)phenyl]cin- noline-3-carboxylate | 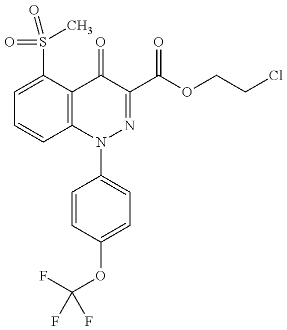 [SMILES: CS(=O)(=O)c1cccc2c1c(=O)c(C(=O)OCCCl)nn2-c1ccc(OC(F)(F)F)cc1] | Rₜ = 2.27 min; MS: m/z = 491 (M + H) |
| P62 | pent-4-ynyl 5- methylsulfonyl-4-oxo-1-[4- (trifluoromethoxy)phenyl]cin- noline-3-carboxylate | 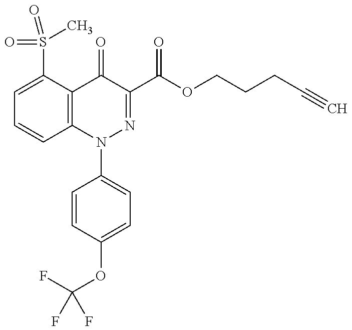 [SMILES: C#CCCCOC(=O)c1nn(-c2ccc(OC(F)(F)F)cc2)c2cccc(S(C)(=O)=O)c2c1=O] | Rₜ = 2.35 min; MS: m/z = 495 (M + H) |
| P63 | cyclopropylmethyl 5- methylsulfonyl-4-oxo-1-[4- (trifluoromethoxy)phenyl]cin- noline-3-carboxylate | 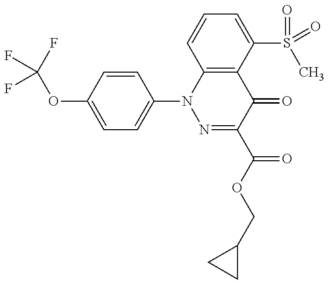 [SMILES: CS(=O)(=O)c1cccc2c1c(=O)c(C(=O)OCC1CC1)nn2-c1ccc(OC(F)(F)F)cc1] | Rₜ = 2.37 min; MS: m/z = 483 (M + H) |
| P64 | 1-methylallyl 5- methylsulfonyl-4-oxo-1-[4- (trifluoromethoxy)phenyl]cin- noline-3-carboxylate | 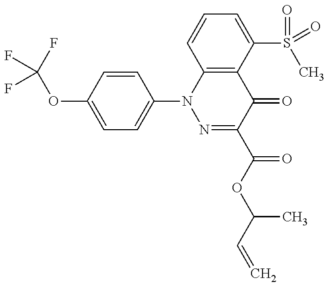 [SMILES: C=CC(C)OC(=O)c1nn(-c2ccc(OC(F)(F)F)cc2)c2cccc(S(C)(=O)=O)c2c1=O] | Rₜ = 2.41 min; MS: m/z = 483 (M + H) |
| P65 | isopropyl 5-methylsulfonyl-4- oxo-1-[4- (trifluoromethoxy)phenyl]cin- noline-3-carboxylate | 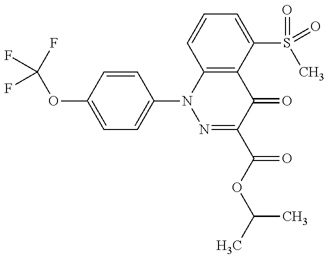 [SMILES: CC(C)OC(=O)c1nn(-c2ccc(OC(F)(F)F)cc2)c2cccc(S(C)(=O)=O)c2c1=O] | Rₜ = 2.35 min; MS: m/z = 471 (M + H) |
| P66 | 2-chloroallyl 5- methylsulfonyl-4-oxo-1-[4- (trifluoromethoxy)phenyl]cin- noline-3-carboxylate | 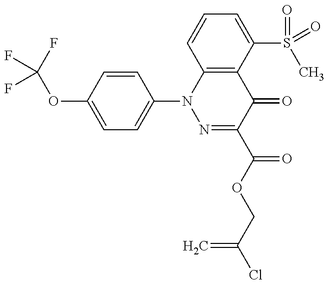 [SMILES: C=C(Cl)COC(=O)c1nn(-c2ccc(OC(F)(F)F)cc2)c2cccc(S(C)(=O)=O)c2c1=O] | Rₜ = 2.41 min; MS: m/z = 503 (M + H) |
| P67 | 2,2-difluoroethyl 5- methylsulfonyl-4-oxo-1-[4- (trifluoromethoxy)phenyl]cin- noline-3-carboxylate | 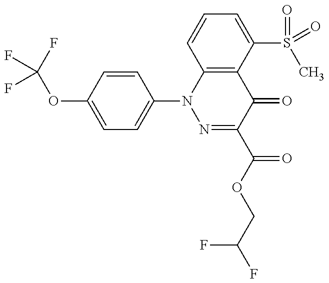 [SMILES: CS(=O)(=O)c1cccc2c1c(=O)c(C(=O)OCC(F)F)nn2-c1ccc(OC(F)(F)F)cc1] | Rₜ = 2.23 min; MS: m/z = 493 (M + H) |
| P68 | 2,2-dimethylpropyl 5- methylsulfonyl-4-oxo-1-[4- (trifluoromethoxy)phenyl]cin- noline-3-carboxylate | 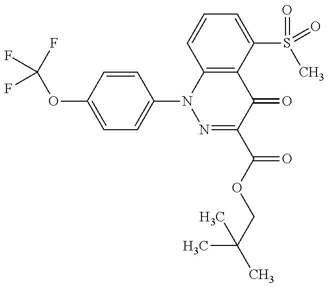 [SMILES: CC(C)(C)COC(=O)c1nn(-c2ccc(OC(F)(F)F)cc2)c2cccc(S(C)(=O)=O)c2c1=O] | Rₜ = 2.61 min; MS: m/z = 499 (M + H) |
| P69 | 3-methoxypropyl 5- methylsulfonyl-4-oxo-1-[4- (trifluoromethoxy)phenyl]cin- noline-3-carboxylate | 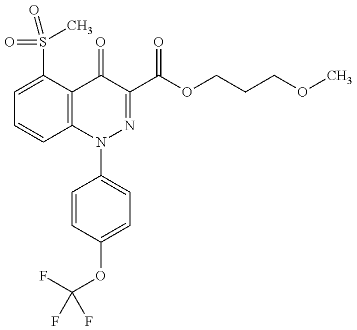 [SMILES: COCCCOC(=O)c1nn(-c2ccc(OC(F)(F)F)cc2)c2cccc(S(C)(=O)=O)c2c1=O] | Rₜ = 2.22 min; MS: m/z = 501 (M + H) |
| P70 | tetrahydrofuran-3-yl 5- methylsulfonyl-4-oxo-1-[4- (trifluoromethoxy)phenyl]cin- noline-3-carboxylate | 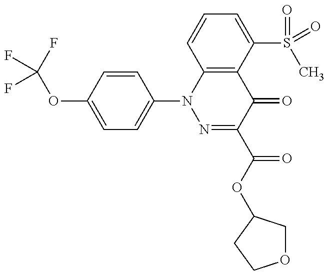 [SMILES: CS(=O)(=O)c1cccc2c1c(=O)c(C(=O)OC1CCOC1)nn2-c1ccc(OC(F)(F)F)cc1] | Rₜ = 2.11 min; MS: m/z = 499 (M + H) |
| P71 | but-3-ynyl 5-methylsulfonyl- 4-oxo-1-[4- (trifluoromethoxy)phenyl]cin- noline-3-carboxylate | 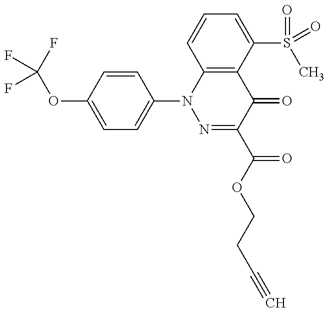 [SMILES: C#CCCOC(=O)c1nn(-c2ccc(OC(F)(F)F)cc2)c2cccc(S(C)(=O)=O)c2c1=O] | Rₜ = 2.25 min; MS: m/z = 481 (M + H) |
| P72 | isobutyl 5-methylsulfonyl-4- oxo-1-[4- (trifluoromethoxy)phenyl]cin- noline-3-carboxylate | 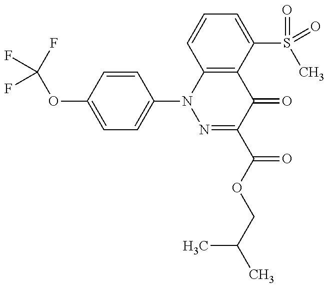 [SMILES: CC(C)COC(=O)c1nn(-c2ccc(OC(F)(F)F)cc2)c2cccc(S(C)(=O)=O)c2c1=O] | Rₜ = 2.50 min; MS: m/z = 485 (M + H) |
| P73 | 2-cyanoethyl 5- methylsulfonyl-4-oxo-1-[4- (trifluoromethoxy)phenyl]cin- noline-3-carboxylate | 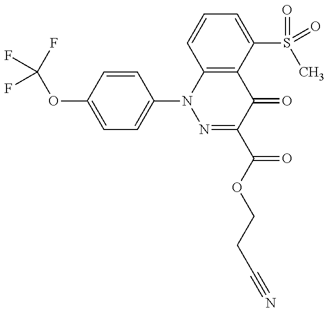 [SMILES: CS(=O)(=O)c1cccc2c1c(=O)c(C(=O)OCCC#N)nn2-c1ccc(OC(F)(F)F)cc1] | Rₜ = 2.06 min; MS: m/z = 482 (M + H) |
| P74 | 1-cyclopropylethyl 5- methylsulfonyl-4-oxo-1-[4- (trifluoromethoxy)phenyl]cin- noline-3-carboxylate | 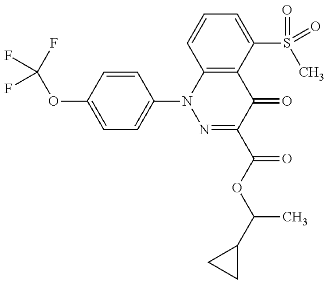 [SMILES: CC(OC(=O)c1nn(-c2ccc(OC(F)(F)F)cc2)c2cccc(S(C)(=O)=O)c2c1=O)C1CC1] | Rₜ = 2.48 min; MS: m/z = 497 (M + H) |
| P75 | penyl 5-methylsulfonyl-4- oxo-1-[4- (trifluoromethoxy)phenyl]cin- noline-3-carboxylate | 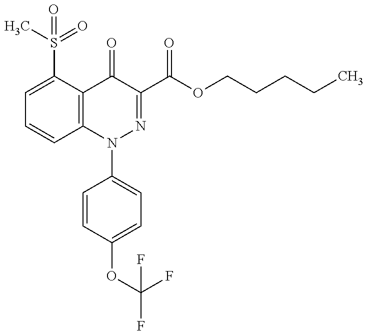 [SMILES: CCCCCOC(=O)c1nn(-c2ccc(OC(F)(F)F)cc2)c2cccc(S(C)(=O)=O)c2c1=O] | Rₜ = 2.62 min; MS: m/z = 499 (M + H) |
| P76 | 2-(dimethylamino)ethyl 5- methylsulfonyl-4-oxo-1-[4- (trifluoromethoxy)phenyl]cin- noline-3-carboxylate | 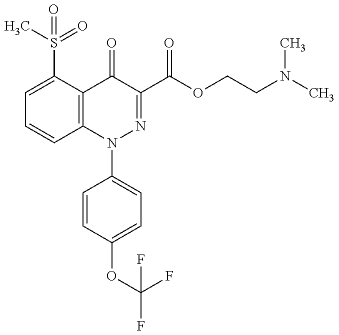 [SMILES: CN(C)CCOC(=O)c1nn(-c2ccc(OC(F)(F)F)cc2)c2cccc(S(C)(=O)=O)c2c1=O] | Rₜ = 1.61 min; MS: m/z = 500 (M + H) |
| P77 | heptyl 5-methylsulfonyl-4- oxo-1-[4- (trifluoromethoxy)phenyl]cin- noline-3-carboxylate | 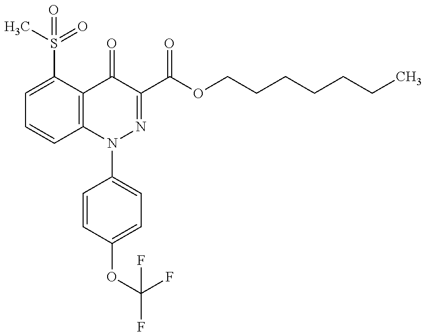 [SMILES: CCCCCCCOC(=O)c1nn(-c2ccc(OC(F)(F)F)cc2)c2cccc(S(C)(=O)=O)c2c1=O] | Rₜ = 2.86 min; MS: m/z = 527 (M + H) |
| P78 | prop-2-ynyl 5- methylsulfonyl-4-oxo-1-[4- (trifluoromethoxy)phenyl]cin- noline-3-carboxylate | 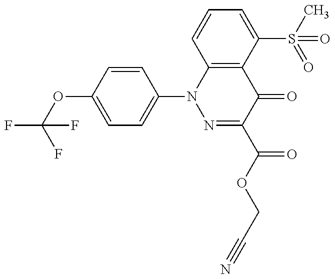 | Rₜ = 2.72 min; MS: m/z = 525 (M + H) |
| P79 | prop-2-ynyl 5- methylsulfonyl-4-oxo-1-[4- (trifluoromethoxy)phenyl]cin- noline-3-carboxylate | 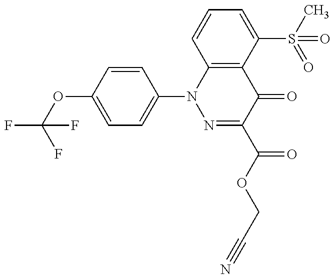 [SMILES: CS(=O)(=O)c1cccc2c1c(=O)c(C(=O)OCC#N)nn2-c1ccc(OC(F)(F)F)cc1] | Rₜ = 2.19 min; MS: m/z = 467 (M + H) |
| P80 | allyl 5-methylsulfonyl-4-oxo- 1-[4- (trifluoromethoxy)phenyl]cin- noline-3-carboxylate | 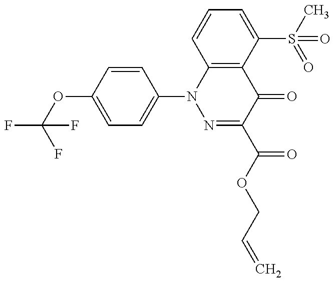 [SMILES: C=CCOC(=O)c1nn(-c2ccc(OC(F)(F)F)cc2)c2cccc(S(C)(=O)=O)c2c1=O] | Rₜ = 2.3 min; MS: m/z = 469 (M + H) |
| P81 | (2-methoxy-2-oxo-ethyl) 5- methylsulfonyl-4-oxo-1-[4- (trifluoromethoxy)phenyl]cin- noline-3-carboxylate | 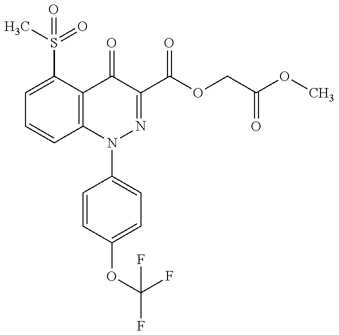 [SMILES: COC(=O)COC(=O)c1nn(-c2ccc(OC(F)(F)F)cc2)c2cccc(S(C)(=O)=O)c2c1=O] | Rₜ = 2.12 min; MS: m/z = 501 (M + H) |
| P82 | nonyl 5-methylsulfonyl-4- oxo-1-[4- (trifluoromethoxy)phenyl]cin- noline-3-carboxylate | 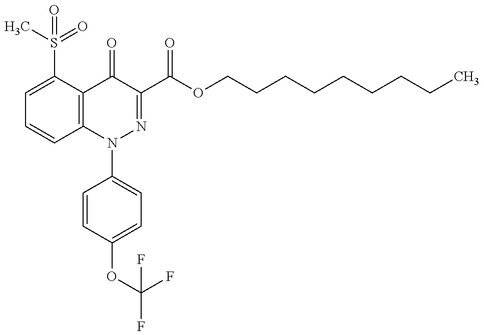 [SMILES: CCCCCCCCCOC(=O)c1nn(-c2ccc(OC(F)(F)F)cc2)c2cccc(S(C)(=O)=O)c2c1=O] | Rₜ = 3.09 min; MS: m/z = 555 (M + H) |
| P83 | 9-phenylnonyl 5- methylsulfonyl-4-oxo-1-[4- (trifluoromethoxy)phenyl]cin- noline-3-carboxylate | 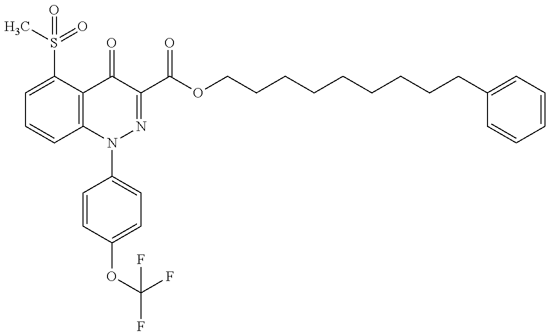 [SMILES: CS(=O)(=O)c1cccc2c1c(=O)c(C(=O)OCCCCCCCCCc1ccccc1)nn2-c1ccc(OC(F)(F)F)cc1] | Rₜ = 3.22 min; MS: m/z = 630 (M + H) |
| P84 | 3-phenylpropyl 5- methylsulfonyl-4-oxo-1-[4- (trifluoromethoxy)phenyl]cin- noline-3-carboxylate | 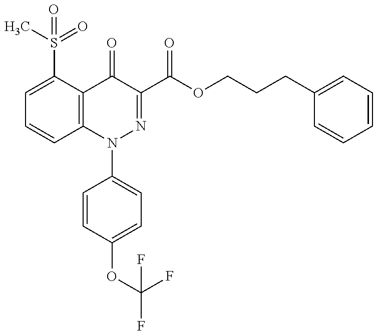 [SMILES: CS(=O)(=O)c1cccc2c1c(=O)c(C(=O)OCCCc1ccccc1)nn2-c1ccc(OC(F)(F)F)cc1] | Rₜ = 2.65 min; MS: m/z = 547 (M + H) |
| P85 | (3-methoxy-3-methyl-butyl) 5-methysulfonyl-4-oxo-1-[4- (trifluoromethoxy)phenyl]cin- noline-3-carboxylate | 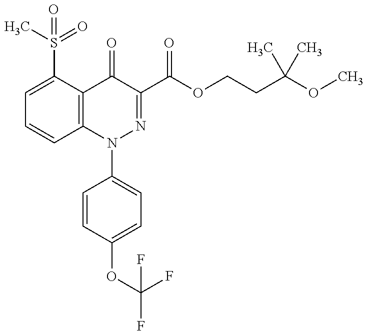 [SMILES: COC(C)(C)CCOC(=O)c1nn(-c2ccc(OC(F)(F)F)cc2)c2cccc(S(C)(=O)=O)c2c1=O] | Rₜ = 2.38 min; MS: m/z = 551 (M + H) |
| P86 | 3,3-dimethylbutyl 5- methylsulfonyl-4-oxo-1-[4- (trifluoromethoxy)phenyl]cin- noline-3-carboxylate | 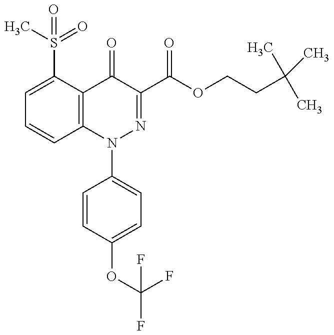 [SMILES: CC(C)(C)CCOC(=O)c1nn(-c2ccc(OC(F)(F)F)cc2)c2cccc(S(C)(=O)=O)c2c1=O] | Rₜ = 2.70 min; MS: m/z = 513 (M + H) |
| P87 | 2-cyclohexylethyl 5- methylsulfonyl-4-oxo-1-[4- (trifluoromethoxy)phenyl]cin- noline-3-carboxylate | 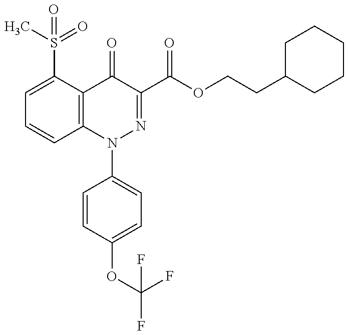 [SMILES: CS(=O)(=O)c1cccc2c1c(=O)c(C(=O)OCCC1CCCCC1)nn2-c1ccc(OC(F)(F)F)cc1] | Rₜ = 2.88 min; MS: m/z = 539 (M + H) |
| P88 | isopentyl 5-methylsulfonyl-4- oxo-1-[4- (trifluoromethoxy)phenyl]cin- noline-3-carboxylate | 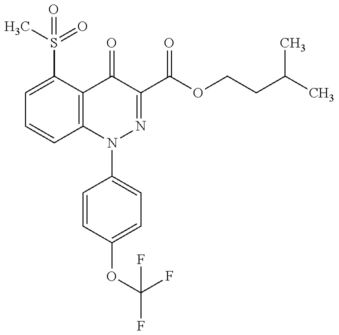 [SMILES: CC(C)CCOC(=O)c1nn(-c2ccc(OC(F)(F)F)cc2)c2cccc(S(C)(=O)=O)c2c1=O] | Rₜ = 2.61 min; MS: m/z = 499 (M + H) |
| P89 | 4-benzyloxybutyl 5- methylsulfonyl-4-oxo-1-[4- (trifluoromethoxy)phenyl]cin- noline-3-carboxylate | 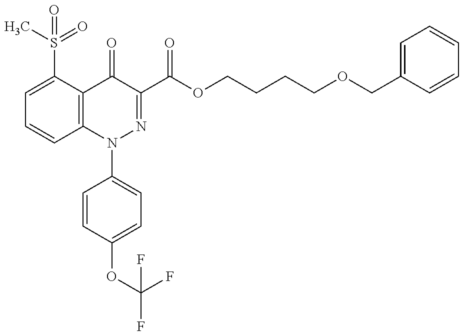 [SMILES: CS(=O)(=O)c1cccc2c1c(=O)c(C(=O)OCCCCOCc1ccccc1)nn2-c1ccc(OC(F)(F)F)cc1] | Rₜ = 2.66 min; MS: m/z = 591 (M + H) |
| P90 | S-octyl 5-methylsulfonyl-4- oxo-1-[4- (trifluoromethoxy)phenyl]cin- noline-3-carbothioate | 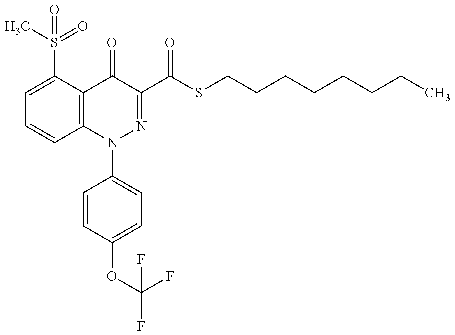 [SMILES: CCCCCCCCSC(=O)c1nn(-c2ccc(OC(F)(F)F)cc2)c2cccc(S(C)(=O)=O)c2c1=O] | Rₜ = 3.12 min; MS: m/z = 557 (M + H) |
| P91 | S-isopentyl 5- methylsulfonyl-4-oxo-1-[4- (trifluoromethoxy)phenyl]cin- noline-3-carbothioate | 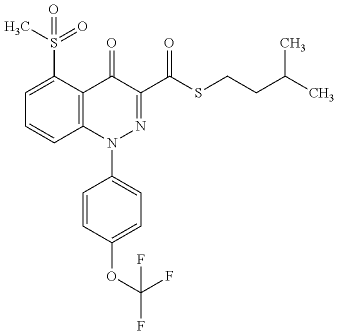 [SMILES: CC(C)CCSC(=O)c1nn(-c2ccc(OC(F)(F)F)cc2)c2cccc(S(C)(=O)=O)c2c1=O] | Rₜ = 2.76 min; MS: m/z = 515 (M + H) |
| P92 | S-(3-phenylpropyl) 5- methyslulfonyl-4-oxo-1-[4- (trifluoromethoxy)phenyl]cin- noline-3-carbothioate | 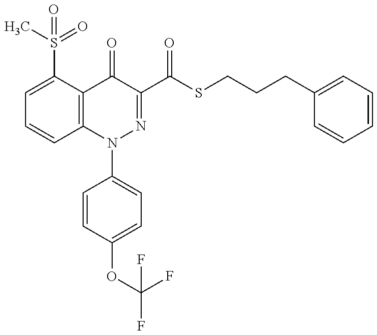 [SMILES: CS(=O)(=O)c1cccc2c1c(=O)c(C(=O)SCCCc1ccccc1)nn2-c1ccc(OC(F)(F)F)cc1] | Rₜ = 2.77 min; MS: m/z = 563 (M + H) |

### Biological Examples

Seeds of a variety of test species are sown in standard soil in pots (Amaranthus retoflexus (AMARE), Solanum nigrum (SOLNI), Setaria faberi (SETFA), Lolium perenne (LOLPE), Echinochloa crus-galli (ECHCG), Ipomoea hederacea (IPOHE), Abutilon theophrasti (ABUTH), Zea mays (ZEAMX)). After 8 days cultivation under controlled conditions in a glasshouse (at 24° C./16° C., day/night; 14 hours light; 65% humidity), the plants are sprayed with an aqueous spray solution derived from the formulation of the technical active ingredient in acetone/water (50:50) solution containing 0.5% Tween 20 (polyoxyethelyene sorbitan monolaurate, CAS RN 9005-64-5). Compounds are applied at 1000 g/ha unless otherwise stated. The test plants are then grown in a glasshouse under controlled conditions in a glasshouse (at 24° C./16° C., day/night; 14 hours light; 65% humidity) and watered twice daily. After 13 days the test is evaluated for the percentage damage caused to the plant. The biological activities are shown in the following table on a five-point scale (5=81-100%; 4=61-80%; 3=41-60%; 2=21-40%; 1=10-20%; 0=0%;−=not tested).

TABLE B1
Pre-emergence Test

| Compound | AMARE | SOLNI | SETFA | LOLPE | ECHCG | IPOHE | ABUTH | ZEAMX |
|---|---|---|---|---|---|---|---|---|
| P1 | 0 | 0 | 0 | 0 | 0 | 0 | — | — |
| P2 | 1 | 4 | 5 | 5 | 5 | 1 | — | — |
| P3 | 1 | 1 | 4 | 4 | 4 | 1 | — | — |
| P4 | 0 | 0 | 0 | 0 | 0 | 0 | — | — |
| P5 | 1 | 1 | 4 | 4 | 4 | 1 | — | — |
| P6 | 1 | — | 4 | — | 3 | 1 | 2 | 1 |
| P7 | 1 | — | 4 | — | 4 | 1 | 1 | 1 |
| P8 | 1 | — | 4 | — | 3 | 1 | 1 | 1 |
| P9 | 1 | — | 4 | — | 4 | 1 | 1 | 1 |
| P10 | 1 | — | 1 | — | 0 | 0 | 1 | 0 |
| P11 | 0 | — | 0 | — | 0 | 0 | 0 | 0 |
| P12 | 0 | — | 4 | — | 1 | 0 | 1 | 0 |
| P13 | 0 | — | 0 | — | 0 | 0 | 1 | 0 |
| P14 | 1 | — | 1 | — | 1 | 0 | 1 | 1 |
| P15 | 1 | — | 1 | — | 1 | 0 | 1 | 0 |
| P18 | 0 | — | 0 | — | 1 | 0 | 0 | 1 |
| P19 | 1 | — | 5 | — | 2 | 0 | 0 | 0 |
| P20 | 0 | — | 0 | — | 0 | 0 | 0 | 0 |
| P21 | 0 | — | 4 | — | 2 | 0 | 0 | 1 |
| P22 | 0 | — | 0 | — | 0 | 0 | 0 | 0 |
| P23 | 0 | — | 0 | — | 0 | 0 | 0 | 1 |
| P24 | 0 | — | 0 | — | 0 | 0 | 0 | 0 |
| P25 | 0 | — | 1 | — | 0 | 0 | 0 | 0 |
| P26 | 1 | — | 0 | — | 0 | 0 | 0 | 0 |
| P27 | 0 | — | 3 | — | 0 | 0 | 0 | 1 |
| P28 | 0 | — | 0 | — | 0 | 0 | 0 | 0 |
| P29 | 0 | — | 0 | — | 0 | 0 | 0 | 0 |
| P30 | 0 | — | 0 | — | 0 | 0 | 0 | 0 |
| P31 | 0 | — | 0 | — | 0 | 0 | 0 | 0 |
| P32 | 0 | — | 2 | — | 1 | 0 | 0 | 0 |
| P33 | 0 | — | 2 | — | 1 | 0 | 0 | 0 |
| P34 | 0 | — | 0 | — | 0 | 0 | 0 | 0 |
| P35 | 0 | — | 0 | — | 0 | 0 | 1 | 1 |
| P37 | 0 | — | 0 | — | 0 | 0 | 1 | 0 |
| P38 | 0 | — | 0 | — | 0 | 0 | 0 | 0 |
| P39 | 1 | — | 0 | — | 0 | 0 | 0 | 0 |
| P40 | 0 | — | 1 | — | 0 | 0 | 1 | 1 |
| P41 | 1 | — | 4 | — | 2 | 1 | 1 | 1 |
| P42 | 0 | — | 4 | — | 3 | 1 | 1 | 0 |
| P43 | 0 | — | 4 | — | 2 | 1 | 0 | 0 |
| P44 | 1 | — | 4 | — | 2 | 0 | 0 | 0 |
| P45 | 0 | — | 0 | — | 0 | 0 | 0 | 0 |
| P46 | 0 | — | 0 | — | 0 | 0 | 1 | 2 |
| P47 | 0 | — | 0 | — | 0 | 0 | 0 | 0 |
| P48 | 0 | — | 0 | — | 0 | 0 | 0 | 0 |
| P49 | 0 | — | 0 | — | 0 | 0 | 0 | 0 |
| P50 | 0 | — | 4 | — | 1 | 0 | 0 | 0 |
| P51 | 0 | — | 0 | — | 0 | 0 | 0 | 0 |
| P52 | 1 | — | 4 | — | 3 | 0 | 1 | 0 |
| P53 | 1 | — | 1 | — | 1 | 0 | 1 | — |
| P54 | 1 | — | 2 | — | 2 | 0 | 1 | 0 |
| P55 | 0 | — | 0 | — | 0 | 0 | 1 | 0 |
| P56 | 0 | — | 0 | — | 0 | 0 | 0 | 0 |
| P57 | 0 | — | 0 | — | 0 | 0 | 0 | 0 |
| P59 | 2 | — | 4 | — | 3 | 2 | 3 | 3 |
| P60 | 1 | — | 4 | — | 1 | 0 | 1 | 1 |
| P61 | 0 | — | 5 | — | 4 | 0 | 0 | 1 |
| P62 | 0 | — | 5 | — | 4 | 0 | 0 | 0 |
| P63 | 0 | — | 5 | — | 3 | 0 | 0 | 0 |
| P64 | 0 | — | 5 | — | 4 | 0 | 0 | 0 |
| P65 | 1 | — | 4 | — | 4 | 0 | 1 | 0 |
| P66 | 0 | — | 5 | — | 4 | 1 | 0 | 1 |
| P67 | 0 | — | 5 | — | 4 | 0 | 0 | 2 |
| P68 | 0 | — | 4 | — | 4 | 0 | 0 | 0 |
| P69 | 0 | — | 4 | — | 4 | 0 | 0 | 0 |
| P70 | 0 | — | 4 | — | 3 | 0 | 0 | 1 |
| P71 | 0 | — | 5 | — | 4 | 0 | 0 | 0 |
| P72 | 0 | — | 4 | — | 4 | 0 | 0 | 0 |
| P73 | 0 | — | 5 | — | 3 | 0 | 0 | 0 |
| P74 | 0 | — | 5 | — | 4 | 0 | 0 | 0 |
| P75 | 0 | — | 4 | — | 4 | 0 | 0 | 1 |
| P76 | 0 | — | 4 | — | 3 | 0 | 1 | 0 |
| P77 | 0 | — | 5 | — | 3 | 0 | 0 | 0 |
| P78 | 0 | — | 5 | — | 4 | 0 | 0 | 0 |
| P79 | 0 | — | 5 | — | 5 | 1 | 0 | 1 |
| P80 | 0 | — | 5 | — | 4 | 0 | 0 | 1 |
| P81 | 0 | — | 5 | — | 4 | 0 | 0 | 0 |
| P82 | 0 | — | 3 | — | 1 | 0 | 0 | 0 |
| P83 | 0 | — | 0 | — | 0 | 0 | 0 | 0 |
| P84 | 0 | — | 4 | — | 3 | 0 | 0 | 0 |
| P85 | 0 | — | 4 | — | 4 | 0 | 0 | 0 |
| P86 | 0 | — | 4 | — | 5 | 0 | 1 | 1 |
| P87 | 0 | — | 3 | — | 1 | 1 | 1 | 1 |
| P88 | 0 | — | 5 | — | 4 | 0 | 0 | 0 |
| P89 | 0 | — | 4 | — | 4 | 0 | 0 | 0 |
| P90 | 0 | — | 4 | — | 3 | 0 | 0 | 0 |
| P91 | 0 | — | 4 | — | 3 | 0 | 0 | 0 |
| P92 | 1 | — | 3 | — | 1 | 0 | 1 | 0 |

TABLE B2
Post-emergence Test

| Cpd No. | AMARE | SOLNI | SETFA | LOLPE | ECHCG | IPOHE | ABUTH | ZEAMX |
|---|---|---|---|---|---|---|---|---|
| P1 | 0 | 0 | 0 | 0 | 0 | 0 | — | — |
| P2 | 1 | 4 | 4 | 4 | 4 | 2 | — | — |
| P3 | 1 | 1 | 4 | 4 | 4 | 1 | — | — |
| P4 | 0 | 0 | 0 | 0 | 0 | 0 | — | — |
| P5 | 1 | 4 | 4 | 4 | 4 | 1 | — | — |
| P6 | 2 | — | 3 | — | 4 | 1 | 1 | 1 |
| P7 | 1 | — | 4 | — | 4 | 2 | 1 | 2 |
| P8 | 1 | — | 4 | — | 4 | 1 | 1 | 1 |
| P9 | 1 | — | 3 | — | 4 | 1 | 1 | 1 |
| P10 | 1 | — | 1 | — | 1 | 0 | 1 | 1 |
| P11 | 0 | — | 1 | — | 0 | 0 | 1 | 1 |
| P12 | 1 | — | 3 | — | 3 | 0 | 1 | 1 |
| P13 | 1 | — | 1 | — | 1 | 0 | 0 | 1 |
| P14 | 1 | — | 0 | — | 0 | 0 | 1 | 0 |
| P15 | 1 | — | 1 | — | 1 | 0 | 1 | 1 |
| P18 | 1 | — | 2 | — | 1 | 0 | 1 | 1 |
| P19 | 1 | — | 3 | — | 2 | 1 | 1 | 1 |
| P20 | 0 | — | 0 | — | 0 | 0 | 0 | 0 |
| P21 | 0 | — | 2 | — | 2 | 0 | 0 | 0 |
| P22 | 1 | — | 1 | — | 1 | 1 | 1 | 0 |
| P23 | 0 | — | 1 | — | 1 | 1 | 1 | 1 |
| P24 | 1 | — | 0 | — | 1 | 0 | 1 | 1 |
| P25 | 0 | — | 2 | — | 1 | 1 | 1 | 1 |
| P26 | 0 | — | 1 | — | 1 | 0 | 1 | 1 |
| P27 | 0 | — | 2 | — | 2 | 0 | 1 | 1 |
| P28 | 1 | — | 1 | — | 0 | 0 | 1 | 0 |
| P29 | 0 | — | 0 | — | 0 | 0 | 0 | 0 |
| P30 | 1 | — | 1 | — | 1 | 1 | 1 | 1 |
| P31 | 0 | — | 0 | — | 0 | 0 | 0 | 0 |
| P32 | 0 | — | 1 | — | 1 | 0 | 0 | 1 |
| P33 | 0 | — | 0 | — | 0 | 0 | 1 | 0 |
| P34 | 0 | — | 0 | — | 0 | 0 | 0 | 0 |
| P35 | 1 | — | 2 | — | 1 | 0 | 1 | 1 |
| P37 | 0 | — | 1 | — | 1 | 1 | 1 | 1 |
| P38 | 1 | — | 1 | — | 0 | 0 | 1 | 1 |
| P39 | 0 | — | 0 | — | 0 | 1 | 1 | 1 |
| P40 | 1 | — | 1 | — | 1 | 0 | 1 | 1 |
| P41 | 1 | — | 4 | — | 4 | 1 | 1 | 1 |
| P42 | 1 | — | 4 | — | 4 | 1 | 1 | 1 |
| P43 | 1 | — | 4 | — | 3 | 1 | 2 | 1 |
| P44 | 1 | — | 4 | — | 4 | 1 | 1 | 1 |
| P45 | 0 | — | 1 | — | 1 | 0 | 0 | 1 |
| P46 | 0 | — | 1 | — | 1 | 1 | 1 | 1 |
| P47 | 0 | — | 1 | — | 1 | 0 | 0 | 0 |
| P48 | 0 | — | 0 | — | 0 | 0 | 0 | 0 |
| P49 | 0 | — | 1 | — | 1 | 1 | 0 | 0 |
| P50 | 2 | — | 2 | — | 0 | 0 | 0 | 0 |
| P51 | 0 | — | 0 | — | 0 | 0 | 0 | 0 |
| P52 | 1 | — | 2 | — | 2 | 0 | 1 | 1 |
| P53 | 0 | — | 0 | — | 1 | 1 | 1 | 0 |
| P54 | 2 | — | 0 | — | 0 | 1 | 1 | 1 |
| P55 | 0 | — | 0 | — | 0 | 0 | 0 | 0 |
| P56 | 1 | — | 0 | — | 0 | 0 | 0 | 0 |
| P57 | 0 | — | 1 | — | 1 | 0 | 0 | 0 |
| P59 | 1 | — | 4 | — | 4 | 3 | 1 | 2 |
| P60 | 1 | — | 1 | — | 1 | 1 | 1 | 1 |
| P61 | 0 | — | 4 | — | 3 | 0 | 2 | 0 |
| P62 | 0 | — | 4 | — | 3 | 0 | 0 | 1 |
| P63 | 0 | — | 4 | — | 3 | 0 | 0 | 0 |
| P64 | 0 | — | 4 | — | 3 | 0 | 1 | 1 |
| P65 | 1 | — | 4 | — | 3 | 0 | 1 | 0 |
| P66 | 0 | — | 4 | — | 4 | 0 | 1 | 1 |
| P67 | 1 | — | 4 | — | 3 | 1 | 1 | 1 |
| P68 | 0 | — | 3 | — | 3 | 0 | 0 | 1 |
| P69 | 0 | — | 4 | — | 3 | 1 | 1 | 2 |
| P70 | 0 | — | 4 | — | 3 | 0 | 1 | 0 |
| P71 | 0 | — | 3 | — | 3 | 1 | 0 | 1 |
| P72 | 0 | — | 4 | — | 3 | 1 | 0 | 1 |
| P73 | 0 | — | 4 | — | 3 | 1 | 0 | 1 |
| P74 | 1 | — | 4 | — | 4 | 0 | 1 | 1 |
| P75 | 0 | — | 4 | — | 3 | 0 | 0 | 0 |
| P76 | 1 | — | 4 | — | 3 | 1 | 2 | 1 |
| P77 | 0 | — | 4 | — | 3 | 0 | 1 | 0 |
| P78 | 1 | — | 3 | — | 3 | 0 | 3 | 0 |
| P79 | 0 | — | 4 | — | 4 | 1 | 1 | 1 |
| P80 | 0 | — | 4 | — | 3 | 1 | 1 | 1 |
| P81 | 1 | — | 4 | — | 3 | 0 | 1 | 1 |
| P82 | 0 | — | 0 | — | 1 | 1 | 1 | 1 |
| P83 | 0 | — | 0 | — | 0 | 0 | 0 | 0 |
| P84 | 0 | — | 4 | — | 2 | 1 | 1 | 0 |
| P85 | 1 | — | 4 | — | 3 | 0 | 1 | 1 |
| P86 | 0 | — | 4 | — | 4 | 0 | 0 | 0 |
| P87 | 1 | — | 1 | — | 1 | 0 | 1 | 0 |
| P88 | 0 | — | 4 | — | 3 | 0 | 1 | 0 |
| P89 | 0 | — | 4 | — | 3 | 0 | 0 | 0 |
| P90 | 0 | — | 4 | — | 3 | 1 | 0 | 1 |
| P91 | 0 | — | 4 | — | 3 | 1 | 1 | 1 |
| P92 | 1 | — | 3 | — | 3 | 2 | 2 | 1 |

1. A compound of formula (I): 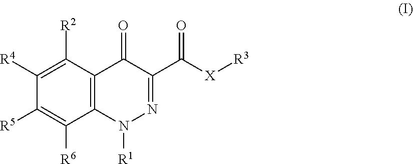
wherein
X is O, NR¹⁰ or S;
R^{r} is phenyl optionally substituted with 1, 2, 3, or 4 groups, which may be the same or different, represented by R⁷;
R² is S(O)C₁-C₆alkyl, S(O)ₙC₁-C₆haloalkyl, or S(O)ₙC₃-C₆cycloalkyl;
n is 0, 1 or 2;
R³ is hydrogen, C₁-C₁₂alkyl, C₁-C₆haloalkyl, cyanoC₁-C₆alkyl, C₃-C₆cycloalkyl, C₃-C₆cycloalkylC₁-C₆alkyl, C₁-C₆alkoxyC₁-C₆alkyl, C₂-C₆alkenyl, C₂-C₆haloalkenyl, C₂-C₆alkynyl, C₁-C₆alkoxycarbonylC₁-C₆alkyl, N,N-di(C₁-C₆alkyl)aminoC₁-C₆alkyl, phenyl, phenylC₁-C₁₂alkyl, benzyloxyC₁-C₆alkyl, heterocyclyl, wherein the wherein the heterocyclyl moiety is a 4-, 5- or 6-membered non-aromatic monocyclic ring comprising 1 or 2 heteroatoms individually selected from N, O and S, and wherein the phenyl and heterocyclyl moieties may be optionally substituted with 1, 2, 3 or 4 groups, which may be the same or different, represented by R¹;
R⁴, R⁵, and R⁶ are each independently selected from hydrogen, halogen, cyano, C₁-C₆alkyl, C₁-C₆alkoxy, C₁-C₆haloalkyl, C₁-C₆haloalkoxy, C₁-C₆alkylsulfanyl, C₁-C₆alkylsulfinyl, and C₁-C₆alkylsulfonyl;
R⁷ is halogen, cyano, C₁-C₆alkyl, C₁-C₆alkoxy, C₁-C₆haloalkyl, C₁-C₆haloalkoxy, C₁-C₆alkylsulfanyl, C₁-C₆alkylsulfinyl, or C₁-C₆alkylsulfonyl; or
any two adjacent R⁷ groups together with the carbon atoms to which they are attached, may form a 5- or 6-membered heterocyclyl ring, comprising 1 or 2 heteroatoms selected from O and N, and wherein the heterocyclyl ring may be optionally substituted with 1, 2, 3, or 4 groups, which may be the same or different, represented by R⁹;
R⁸ and R⁹ are each independently selected from halogen, C₁-C₃alkyl, and C₁-C₃alkoxy;
R¹⁰ is hydrogen, C₁-C₃alkyl, or C₁-C₃alkoxy;
or a salt or an N-oxide thereof.

2. The compound according to claim 1, wherein R^{r} is phenyl optionally substituted with 1 or 2 groups, which may be the same or different, represented by R⁷.

3. The compound according to claim 1, wherein R² is S(O)ₙC₁-C₃alkyl, S(O)ₙC₁-C₃haloalkyl, or S(O)ₙC₃-C₄cycloalkyl.

4. The compound according to claim 1, wherein R² is methylsulfanyl, methylsulfonyl, ethylsulfanyl, ethylsulfonyl, 2,2,2-trifluoroethylsulfanyl, 2,2,2-trifluoroethylsulfonyl, cyclopropylsulfanyl, or cyclopropylsulfonyl.

5. The compound according to claim 1, wherein R³ is hydrogen, C₁-C₁₁alkyl, 2-chloroethyl, 2,2-difluoroethyl, 2-cyanoethyl, cyclopropylmethyl, 1-cyclopropylethyl, 3-methoxypropyl, 3-methoxy-3-methylbutyl, allyl, 1-methylallyl, 2-chloroallyl, prop-2-ynyl, but-3-ynyl, pent-4-ynyl, methoxycarbonylmethyl, N,N-di(methyl)aminoethyl, phenylC₃-C₉alkyl, benzyloxybutyl, or heterocyclyl, wherein the wherein the heterocyclyl moiety is a 5- or 6-membered non-aromatic monocyclic ring comprising a single oxygen atom.

6. The compound according to claim 1, wherein R⁴, R⁵, and R⁶ are each independently selected from hydrogen, fluoro, bromo, cyano, methyl, isopropyl, isobutyl, methoxy, and trifluoromethyl.

7. The compound according to claim 1, wherein R⁴, R⁵, and R⁶ are all hydrogen.

8. The compound according to claim 1, wherein R⁷ is halogen, cyano, C₁-C₃alkyl, C₁-C₃alkoxy, C₁-C₃haloalkyl, C₁-C₃haloalkoxy, C₁-C₃alkylsulfanyl, C₁-C₃alkylsulfinyl, or C₁-C₃alkylsulfonyl.

9. The compound according to claim 1, wherein R⁷ is fluoro, bromo, chloro, cyano, methyl, methoxy, trifluoromethyl, or trifluoromethoxy.

10. The compound according to claim 1, wherein X is O.

11. A herbicidal composition comprising a compound according to claim 1 and an agriculturally acceptable formulation adjuvant.

12. A herbicidal composition according to claim 11, further comprising at least one additional pesticide.

13. A herbicidal composition according to claim 12, wherein the additional pesticide is a herbicide or herbicide safener.

14. A method of controlling unwanted plant growth, comprising applying a compound of Formula (I) as defined in claim 1 to the unwanted plants or to the locus thereof.

15. Use of a compound of Formula (I) according to claim 1 as a herbicide.
